SEQUENCE LISTING (206 sequences)

SEQ ID NO: 1 - DNA, 2103 bp, Artificial Sequence
  Synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctggtggagtctgggggaggcttggtccagcctggggggtccctgagactctcctgtacagcctctggattctccctcagtagctatgcaatgagctgggtccgccaggctccagggaaggggctggagtacatcggctacattggtgatactactggcatagcctacgcgagctgggcgaatggcagattcaccatctccaaagacaataccaagaacacggtggatcttcaaatgaacagcctgagagccgaggacacggctgtgtattactgtgcgagaggctggtcctacttagacatctggggccaagggaccctggtcaccgtctcgagcggtggaggcggatctggcggaggtggttccggcggtggcggctccggtggaggcggctctgcccttgtgatgacccagtctccatcctccctgtctgcatctgtaggagacagagtcaccatcacttgccaggccagtcagaacatttacagcaatttagcctggtatcagcagaaaccagggaaagttcctaagctcctgatctatcaggcctccactctggcatctggggtcccatctcggttcagtggcagtggatatgggacagatttcactctcaccatcagcagcctgcagcctgaagatgttgcaacttattactgtcaaggcggttattatagtgctgcccttaatactttcggcggagggaccaaggtggagatcaaa

SEQ ID NO: 2 - protein, 701 aa, Artificial Sequence
  Synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCTASGFSLSSYAMSWVRQAPGKGLEYIGYIGDTTGIAYASWANGRFTISKDNTKNTVDLQMNSLRAEDTAVYYCARGWSYLDIWGQGTLVTVSSGGGGSGGGGSGGGGSGGGGSALVMTQSPSSLSASVGDRVTITCQASQNIYSNLAWYQQKPGKVPKLLIYQASTLASGVPSRFSGSGYGTDFTLTISSLQPEDVATYYCQGGYYSAALNTFGGGTKVEIK

SEQ ID NO: 3 - DNA, 645 bp, Artificial Sequence
  synthesized
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 4 - protein, 215 aa, Artificial Sequence
  synthesized
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 2106 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccagtcggtggaggagtctgggggaggcttggtccagcctggggggtccctgagactctcctgtaccgcctctggaatcgaccttaatacctacgacatgatctgggtccgccaggctccaggcaaggggctagagtgggttggaatcattacttatagtggtagtagatactacgcgaactgggcgaaaggccgattcaccatctccaaagacaataccaagaacacggtgtatctgcaaatgaacagcctgagagctgaggacacggctgtgtattactgtgcgagagattatatgagtggttcccacttgtggggccagggaaccctggtcaccgtctcttccggtggaggcggttcaggcggaggtggaagtggtggtggcggctctggaggcggcggatctgcctatgatatgacccagtctccatcttccgtgtctgcatctgtaggagacagagtcaccatcaagtgtcaggccagtgaggacatttatagcttcttggcctggtatcagcagaaaccagggaaagcccctaagctcctgatccattctgcatcctctctggcatctggggtcccatcaaggttcagcggcagtggatctgggacagatttcactctcaccatcagcagcctgcagcctgaagattttgcaacttactattgtcaacagggttatggtaaaaataatgttgataatgctttcggcggagggaccaaggtggagatcaaa

SEQ ID NO: 6 - protein, 702 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQSVEESGGGLVQPGGSLRLSCTASGIDLNTYDMIWVRQAPGKGLEWVGIITYSGSRYYANWAKGRFTISKDNTKNTVYLQMNSLRAEDTAVYYCARDYMSGSHLWGQGTLVTVSSGGGGSGGGGSGGGGSGGGGSAYDMTQSPSSVSASVGDRVTIKCQASEDIYSFLAWYQQKPGKAPKLLIHSASSLASGVPSRFSGSGSGTDFTLTISSLQPEDFATYYCQQGYGKNNVDNAFGGGTKVEIK

SEQ ID NO: 7 - DNA, 2127 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaa

SEQ ID NO: 8 - protein, 709 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIK

SEQ ID NO: 9 - DNA, 2109 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcagctggtggagtctgggggaggcctggtccagcctgggggctccctgagactctcctgtacaacttctggaatcgaccttagtacctacgacatgatctgggtccgccaggctccaggcaaggggctagagtgggtgggaatcattagttatgttggtaacacatactacgcgagctgggcgaaaggccgattcaccctctccaaagacaatacctcgaccacggtggatctgcaaatgaacagcctgagagctgaggacacggctgtgtattactgtgcgagagattttattagtggttcccacttgtggggccagggaaccctggtcaccgtctcgagcggtggaggcggatctggcggaggtggttccggcggtggcggctccggtggaggcggttctgcctatgatatgacccagtctccatcttccgtgtctgcatctgtaggagacagagtcaccatcaattgtcaggccagtgagagcattagtagcttcttatcctggtatcagcagaaaccagggaaagcccctaagctcctgatcttttctgcatccactctggcatctggggtcccatcaaggttcagcggcagtggatctgggacagatttcactctcaccatcagcagcctgcagcctgaagattttgcaacttactattgtcaacagggttatagtaaaagtaatgtggataatgctttcggcggagggaccaaggtggagatcaaa

SEQ ID NO: 10 - protein, 703 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGLVQPGGSLRLSCTTSGIDLSTYDMIWVRQAPGKGLEWVGIISYVGNTYYASWAKGRFTLSKDNTSTTVDLQMNSLRAEDTAVYYCARDFISGSHLWGQGTLVTVSSGGGGSGGGGSGGGGSGGGGSAYDMTQSPSSVSASVGDRVTINCQASESISSFLSWYQQKPGKAPKLLIFSASTLASGVPSRFSGSGSGTDFTLTISSLQPEDFATYYCQQGYSKSNVDNAFGGGTKVEIK

SEQ ID NO: 11 - DNA, 2121 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcagaggtgcagctggtggagtctgggggaggcttggtccagcctggggggtccctgagactctcctgtgcagcctctggattctccctcagtagctactggatgagctgggtccgccaggctccagggaaggggctggagtggatcggagtcattgatactaatgtttatatatactacgcgaactgggcaaaaggcagattcaccatctccagagacaattccaagaacacgctgtatcttcaaatgaacagcctgagagccgaggacacggctgtgtattactgtgcgagatatgtgggtaataatgatgattatattaacttgtggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccagtccagtcagagtgtttataatggctactggttatcctggtatcagcagaaaccagggaaagcccctaagctcctgatctatggtgcatccactctggcatctggggtcccatcaaggttcagcggcagtggatctgggacagaattcactctcaccatcagcagcctgcagcctgatgattttgcaacttattactgcctaggcagttatactagtagtactgagaactctttcggcggagggaccaaggtggagatcaaa

SEQ ID NO: 12 - protein, 707 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCAASGFSLSSYWMSWVRQAPGKGLEWIGVIDTNVYIYYANWAKGRFTISRDNSKNTLYLQMNSLRAEDTAVYYCARYVGNNDDYINLWGQGTLVTVSSGGGGSGGGGSGGGGSGGGGSDIQMTQSPSTLSASVGDRVTITCQSSQSVYNGYWLSWYQQKPGKAPKLLIYGASTLASGVPSRFSGSGSGTEFTLTISSLQPDDFATYYCLGSYTSSTENSFGGGTKVEIK

SEQ ID NO: 13 - DNA, 2112 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcagaggtgcagctggtggagtctgggggaggcttggtccagcctggggggtccctgagactctcctgtacagtgtctggaatcgacctcagtgtcatcaatatgggctgggtccgccaggctccagggaaggggctggagtggatcggaaccattacttatgttggtaacacatattacgcgagctgggcgaaaggcagattcaccatctccaaaacctcgaccacggtggatcttaaaatcaccagtccgacaaccgaggacacggctgtgtattactgtgcgagagaatctggtactatttattacagttactttaacttgtggggccaagggaccctggtcaccgtctcgagcggtggaggcggatctggcggaggtggttccggcggtggcggctccggtggaggcggctctgcattcgaattgacccagtctccatcctccctgtctgcatctgtaggagacagagtcaccatcaagtgccaggccagtgaaagcattagcaactacttatcctggtatcagcagattccagggaaagttcctaagctcctgatctattatgcatccaatctggcatctggggtcccatctcggttcagtggcagtggatctgggacagatttcactctcaccatcagcagcctgcagcctgaagatgttgcaacttattactgtcaaagctattatggtggtggtagtgcctatactttcggcggagggaccaaggtggagatcaaa

SEQ ID NO: 14 - protein, 704 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCTVSGIDLSVINMGWVRQAPGKGLEWIGTITYVGNTYYASWAKGRFTISKTSTTVDLKITSPTTEDTAVYYCARESGTIYYSYFNLWGQGTLVTVSSGGGGSGGGGSGGGGSGGGGSAFELTQSPSSLSASVGDRVTIKCQASESISNYLSWYQQIPGKVPKLLIYYASNLASGVPSRFSGSGSGTDFTLTISSLQPEDVATYYCQSYYGGGSAYTFGGGTKVEIK

SEQ ID NO: 15 - DNA, 2121 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcagaggtgcagctggtggagtctgggggaggcttggtccagcctggggggtccctgagactctcctgtgcagcctctggattcgacttcagtagcggctactggatatgctgggtccgccaggctccagggaaggggctggagttgatcgcatgcatttatgctggtactagtggtagtacttcctacgcgagctgggcgagaggcagattcaccatctccgaaacctccaagaacacggtgactcttcaaatgaacagcctgagagccgaggactcggctgtgtattactgtgcgagaaatctttacacttacaatagcttgtggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccagtccagtcagagtgtttatgataacaactggttagcctggtatcagcagaaaccagggaaagcccctaagctcctgatctatacagtatccactctggcatctggggtcccatcaaggttcagcggcagtggatctgggacagaattcactctcaccatcagcagcctgcagcctgatgattttgcaacttattactgccaaggcacttattatagtagtggttggaactttgctttcggcggagggaccaaggtggagatcaaa

SEQ ID NO: 16 - protein, 707 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCAASGFDFSSGYWICWVRQAPGKGLELIACIYAGTSGSTSYASWARGRFTISETSKNTVTLQMNSLRAEDSAVYYCARNLYTYNSLWGQGTLVTVSSGGGGSGGGGSGGGGSGGGGSDIQMTQSPSTLSASVGDRVTITCQSSQSVYDNNWLAWYQQKPGKAPKLLIYTVSTLASGVPSRFSGSGSGTEFTLTISSLQPDDFATYYCQGTYYSSGWNFAFGGGTKVEIK

SEQ ID NO: 17 - DNA, 2100 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggagcagctgttggagtctgggggaggcttggtacagcctggggggtccctgagactctcctgtacagcctctggattctcctttagcagctactggatgtgctgggtccgccaggctccagggaaggggctggagtggatcggatgcattacgactggtagtggtagcacttactacgcgagctgggcgaagcgccggttcaccatctccaaagacaattccaagaacacggtgactctgcaaatgaacagcctgagagccgaggacacggccgtatattactgtacgagagcatttgacttgtggggccagggaaccctggtcaccgtctcgagcggtggaggcggatctggcggaggtggttccggcggtggcggctccggtggaggcggctctgacatccagatgacccagtctccatcctccctgtctgcatctgtaggagacagagtcaccatcacttgccaggccagtcagagcatttacagctacttaaactggtatcagcagaaaccagggaaagcccctaagctcctgatctatggtgcatccaatctggcatctggggtcccatcaaggttcagtggcagtggatctgggacagatttcactctcaccatcagcagtctgcaacctgaagattttgcaacttactactgtcaaagcagttggttgagtggtgctgttggtaatgctttcggcggagggaccaaggtggagatcaaa

SEQ ID NO: 18 - protein, 700 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQEQLLESGGGLVQPGGSLRLSCTASGFSFSSYWMCWVRQAPGKGLEWIGCITTGSGSTYYASWAKRRFTISKDNSKNTVTLQMNSLRAEDTAVYYCTRAFDLWGQGTLVTVSSGGGGSGGGGSGGGGSGGGGSDIQMTQSPSSLSASVGDRVTITCQASQSIYSYLNWYQQKPGKAPKLLIYGASNLASGVPSRFSGSGSGTDFTLTISSLQPEDFATYYCQSSWLSGAVGNAFGGGTKVEIK

SEQ ID NO: 19 - DNA, 2097 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggagcaggtgaaggagaccgggggaggcttggtacagcctggggggtccctgagactctcctgtgcagcctctggattcaccatcagcagctatggagtgagctgggtccgccaggctccagggaaggggctggagtgggtcgcattgatttttcccgggattggtttcaaagactacgcgagctgggtgaatggccggttcaccctctccagcgacaacgcccagaacactgtggaactgcaaatgaacagcctgagagccgaggacacggccgtatattactgtgcgagagatttggacttgtggggccagggaaccctggtcaccgtctcgagcggtggaggcggatctggcggaggtggttccggcggtggcggctccggtggaggcggctctgacatccagatgacccagtctccatcctccctgtctgcatctgtaggagacagagtcaccatcacttgccagtccagtccgagtgtttatagtaactacttatcctggtatcagcagaaaccagggaaagttcctaagctcctgatctattatgcatccactctggcatctggggtcccatctcggttcagtggcagtggatctgggacagatttcactctcaccatcagcagcctgcagcctgaagatgttgcaacttattactgtgcaggcggttatagtagtagtactcgtgctttcggcggagggaccaaggtggagatcaaa

SEQ ID NO: 20 - protein, 699 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQEQVKETGGGLVQPGGSLRLSCAASGFTISSYGVSWVRQAPGKGLEWVALIFPGIGFKDYASWVNGRFTLSSDNAQNTVELQMNSLRAEDTAVYYCARDLDLWGQGTLVTVSSGGGGSGGGGSGGGGSGGGGSDIQMTQSPSSLSASVGDRVTITCQSSPSVYSNYLSWYQQKPGKVPKLLIYYASTLASGVPSRFSGSGSGTDFTLTISSLQPEDVATYYCAGGYSSSTRAFGGGTKVEIK

SEQ ID NO: 21 - DNA, 2130 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcagaggtgcagctgttggagtctgggggaggcttggtacagcctggggggtccctgagactctcctgtgcagcctctggattcacctttagcagcagccactggatatgctgggtccgccaggctccagggaaggggctggagtggatcgcatgcatttatactggtagtattgatgtcttttactacgcgagctgggcgaaaggccggttcaccatctccagagacaattccaagaacacgctgtatctgcaaatgaacagcctgagagccgaggacacggccgtatattactgtgcgagagccgctaatactgatactacctactttaacttgtggggccagggaaccctggtcaccgtctcgagcggtggaggcggatctggcggaggtggttccggcggtggcggctccggtggaggcggctctgcctatgatatgacccagtctccatcctccctgtctgcatctgtaggagacagagtcaccatcaattgccaggccagtcagagcattaacaaccaactatcctggtatcagcagaaaccagggaaagttcctaagctcctgatctatggtgcatccactctggcatctggggtcccatctcggttcaccggcagtggatctgggacagatttcactctcaccatcagcagcctgcagcctgaagatgttgcaacttattactgtcatgttcattattgcagtggtggtagttgtttttgggctttcggcggagggaccaaggtggagatcaaa

SEQ ID NO: 22 - protein, 710 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFTFSSSHWICWVRQAPGKGLEWIACIYTGSIDVFYYASWAKGRFTISRDNSKNTLYLQMNSLRAEDTAVYYCARAANTDTTYFNLWGQGTLVTVSSGGGGSGGGGSGGGGSGGGGSAYDMTQSPSSLSASVGDRVTINCQASQSINNQLSWYQQKPGKVPKLLIYGASTLASGVPSRFTGSGSGTDFTLTISSLQPEDVATYYCHVHYCSGGSCFWAFGGGTKVEIK

SEQ ID NO: 23 - DNA, 60 bp, Artificial Sequence
  synthesized
ggtggaggcggatctggcggaggtggttccggcggtggcggctccggtggaggcggctct

SEQ ID NO: 24 - protein, 20 aa, Artificial Sequence
  synthesized
GGGGSGGGGSGGGGSGGGGS

SEQ ID NO: 25 - DNA, 2100 bp, Artificial Sequence
  synthesized
cagtcggtggaggagtctgggggaggcttggtccagcctggggggtccctgagactctcctgtaccgcctctggaatcgaccttaatacctacgacatgatctgggtccgccaggctccaggcaaggggctagagtgggttggaatcattacttatagtggtagtagatactacgcgaactgggcgaaaggccgattcaccatctccaaagacaataccaagaacacggtgtatctgcaaatgaacagcctgagagctgaggacacggctgtgtattactgtgcgagagattatatgagtggttcccacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggttcaggcggaggtggaagtggtggtggcggctctggaggcggcggatctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 26 - protein, 700 aa, Artificial Sequence
  synthesized
QSVEESGGGLVQPGGSLRLSCTASGIDLNTYDMIWVRQAPGKGLEWVGIITYSGSRYYANWAKGRFTISKDNTKNTVYLQMNSLRAEDTAVYYCARDYMSGSHLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 27 - DNA, 651 bp, Artificial Sequence
  synthesized
gcctatgatatgacccagtctccatcttccgtgtctgcatctgtaggagacagagtcaccatcaagtgtcaggccagtgaggacatttatagcttcttggcctggtatcagcagaaaccagggaaagcccctaagctcctgatccattctgcatcctctctggcatctggggtcccatcaaggttcagcggcagtggatctgggacagatttcactctcaccatcagcagcctgcagcctgaagattttgcaacttactattgtcaacagggttatggtaaaaataatgttgataatgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 28 - protein, 217 aa, Artificial Sequence
  synthesized
AYDMTQSPSSVSASVGDRVTIKCQASEDIYSFLAWYQQKPGKAPKLLIHSASSLASGVPSRFSGSGSGTDFTLTISSLQPEDFATYYCQQGYGKNNVDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 2100 bp, Artificial Sequence
  synthesized
gaggtgcagctggtggagtctgggggaggcttggtccagcctggggggtccctgagactctcctgtacagcctctggattctccctcagtagctatgcaatgagctgggtccgccaggctccagggaaggggctggagtacatcggctacattggtgatactactggcatagcctacgcgagctgggcgaatggcagattcaccatctccaaagacaataccaagaacacggtggatcttcaaatgaacagcctgagagccgaggacacggctgtgtattactgtgcgagaggctggtcctacttagacatctggggccaagggaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 30 - protein, 700 aa, Artificial Sequence
  synthesized
EVQLVESGGGLVQPGGSLRLSCTASGFSLSSYAMSWVRQAPGKGLEYIGYIGDTTGIAYASWANGRFTISKDNTKNTVDLQMNSLRAEDTAVYYCARGWSYLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 31 - DNA, 648 bp, Artificial Sequence
  synthesized
gcccttgtgatgacccagtctccatcctccctgtctgcatctgtaggagacagagtcaccatcacttgccaggccagtcagaacatttacagcaatttagcctggtatcagcagaaaccagggaaagttcctaagctcctgatctatcaggcctccactctggcatctggggtcccatctcggttcagtggcagtggatatgggacagatttcactctcaccatcagcagcctgcagcctgaagatgttgcaacttattactgtcaaggcggttattatagtgctgcccttaatactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 32 - protein, 216 aa, Artificial Sequence
  synthesized
ALVMTQSPSSLSASVGDRVTITCQASQNIYSNLAWYQQKPGKVPKLLIYQASTLASGVPSRFSGSGYGTDFTLTISSLQPEDVATYYCQGGYYSAALNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 2121 bp, Artificial Sequence
  synthesized
gaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 34 - protein, 707 aa, Artificial Sequence
  synthesized
EVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 35 - DNA, 651 bp, Artificial Sequence
  synthesized
gacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 36 - protein, 217 aa, Artificial Sequence
  synthesized
DIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 2103 bp, Artificial Sequence
  synthesized
caggtgcagctggtggagtctgggggaggcctggtccagcctgggggctccctgagactctcctgtacaacttctggaatcgaccttagtacctacgacatgatctgggtccgccaggctccaggcaaggggctagagtgggtgggaatcattagttatgttggtaacacatactacgcgagctgggcgaaaggccgattcaccctctccaaagacaatacctcgaccacggtggatctgcaaatgaacagcctgagagctgaggacacggctgtgtattactgtgcgagagattttattagtggttcccacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 38 - protein, 701 aa, Artificial Sequence
  synthesized
QVQLVESGGGLVQPGGSLRLSCTTSGIDLSTYDMIWVRQAPGKGLEWVGIISYVGNTYYASWAKGRFTLSKDNTSTTVDLQMNSLRAEDTAVYYCARDFISGSHLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 39 - DNA, 651 bp, Artificial Sequence
  synthesized
gcctatgatatgacccagtctccatcttccgtgtctgcatctgtaggagacagagtcaccatcaattgtcaggccagtgagagcattagtagcttcttatcctggtatcagcagaaaccagggaaagcccctaagctcctgatcttttctgcatccactctggcatctggggtcccatcaaggttcagcggcagtggatctgggacagatttcactctcaccatcagcagcctgcagcctgaagattttgcaacttactattgtcaacagggttatagtaaaagtaatgtggataatgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 40 - protein, 217 aa, Artificial Sequence
  synthesized
AYDMTQSPSSVSASVGDRVTINCQASESISSFLSWYQQKPGKAPKLLIFSASTLASGVPSRFSGSGSGTDFTLTISSLQPEDFATYYCQQGYSKSNVDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 2112 bp, Artificial Sequence
  synthesized
gaggtgcagctggtggagtctgggggaggcttggtccagcctggggggtccctgagactctcctgtgcagcctctggattctccctcagtagctactggatgagctgggtccgccaggctccagggaaggggctggagtggatcggagtcattgatactaatgtttatatatactacgcgaactgggcaaaaggcagattcaccatctccagagacaattccaagaacacgctgtatcttcaaatgaacagcctgagagccgaggacacggctgtgtattactgtgcgagatatgtgggtaataatgatgattatattaacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 42 - protein, 704 aa, Artificial Sequence
  synthesized
EVQLVESGGGLVQPGGSLRLSCAASGFSLSSYWMSWVRQAPGKGLEWIGVIDTNVYIYYANWAKGRFTISRDNSKNTLYLQMNSLRAEDTAVYYCARYVGNNDDYINLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 43 - DNA, 654 bp, Artificial Sequence
  synthesized
gacatccagatgacccagtctccttccaccctgtctgcatctgtaggagacagagtcaccatcacttgccagtccagtcagagtgtttataatggctactggttatcctggtatcagcagaaaccagggaaagcccctaagctcctgatctatggtgcatccactctggcatctggggtcccatcaaggttcagcggcagtggatctgggacagaattcactctcaccatcagcagcctgcagcctgatgattttgcaacttattactgcctaggcagttatactagtagtactgagaactctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 44 - protein, 218 aa, Artificial Sequence
  synthesized
DIQMTQSPSTLSASVGDRVTITCQSSQSVYNGYWLSWYQQKPGKAPKLLIYGASTLASGVPSRFSGSGSGTEFTLTISSLQPDDFATYYCLGSYTSSTENSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 2109 bp, Artificial Sequence
  synthesized
gaggtgcagctggtggagtctgggggaggcttggtccagcctggggggtccctgagactctcctgtacagtgtctggaatcgacctcagtgtcatcaatatgggctgggtccgccaggctccagggaaggggctggagtggatcggaaccattacttatgttggtaacacatattacgcgagctgggcgaaaggcagattcaccatctccaaaacctcgaccacggtggatcttaaaatcaccagtccgacaaccgaggacacggctgtgtattactgtgcgagagaatctggtactatttattacagttactttaacttgtggggccaagggaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 46 - protein, 703 aa, Artificial Sequence
  synthesized
EVQLVESGGGLVQPGGSLRLSCTVSGIDLSVINMGWVRQAPGKGLEWIGTITYVGNTYYASWAKGRFTISKTSTTVDLKITSPTTEDTAVYYCARESGTIYYSYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 47 - DNA, 648 bp, Artificial Sequence
  synthesized
gcattcgaattgacccagtctccatcctccctgtctgcatctgtaggagacagagtcaccatcaagtgccaggccagtgaaagcattagcaactacttatcctggtatcagcagattccagggaaagttcctaagctcctgatctattatgcatccaatctggcatctggggtcccatctcggttcagtggcagtggatctgggacagatttcactctcaccatcagcagcctgcagcctgaagatgttgcaacttattactgtcaaagctattatggtggtggtagtgcctatactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 48 - protein, 216 aa, Artificial Sequence
  synthesized
AFELTQSPSSLSASVGDRVTIKCQASESISNYLSWYQQIPGKVPKLLIYYASNLASGVPSRFSGSGSGTDFTLTISSLQPEDVATYYCQSYYGGGSAYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 2109 bp, Artificial Sequence
  synthesized
gaggtgcagctggtggagtctgggggaggcttggtccagcctggggggtccctgagactctcctgtgcagcctctggattcgacttcagtagcggctactggatatgctgggtccgccaggctccagggaaggggctggagttgatcgcatgcatttatgctggtactagtggtagtacttcctacgcgagctgggcgagaggcagattcaccatctccgaaacctccaagaacacggtgactcttcaaatgaacagcctgagagccgaggactcggctgtgtattactgtgcgagaaatctttacacttacaatagcttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 50 - protein, 703 aa, Artificial Sequence
  synthesized
EVQLVESGGGLVQPGGSLRLSCAASGFDFSSGYWICWVRQAPGKGLELIACIYAGTSGSTSYASWARGRFTISETSKNTVTLQMNSLRAEDSAVYYCARNLYTYNSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 51 - DNA, 657 bp, Artificial Sequence
  synthesized
gacatccagatgacccagtctccttccaccctgtctgcatctgtaggagacagagtcaccatcacttgccagtccagtcagagtgtttatgataacaactggttagcctggtatcagcagaaaccagggaaagcccctaagctcctgatctatacagtatccactctggcatctggggtcccatcaaggttcagcggcagtggatctgggacagaattcactctcaccatcagcagcctgcagcctgatgattttgcaacttattactgccaaggcacttattatagtagtggttggaactttgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 52 - protein, 219 aa, Artificial Sequence
  synthesized
DIQMTQSPSTLSASVGDRVTITCQSSQSVYDNNWLAWYQQKPGKAPKLLIYTVSTLASGVPSRFSGSGSGTEFTLTISSLQPDDFATYYCQGTYYSSGWNFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 2094 bp, Artificial Sequence
  synthesized
caggagcagctgttggagtctgggggaggcttggtacagcctggggggtccctgagactctcctgtacagcctctggattctcctttagcagctactggatgtgctgggtccgccaggctccagggaaggggctggagtggatcggatgcattacgactggtagtggtagcacttactacgcgagctgggcgaagcgccggttcaccatctccaaagacaattccaagaacacggtgactctgcaaatgaacagcctgagagccgaggacacggccgtatattactgtacgagagcatttgacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 54 - protein, 698 aa, Artificial Sequence
  synthesized
QEQLLESGGGLVQPGGSLRLSCTASGFSFSSYWMCWVRQAPGKGLEWIGCITTGSGSTYYASWAKRRFTISKDNSKNTVTLQMNSLRAEDTAVYYCTRAFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 55 - DNA, 651 bp, Artificial Sequence
  synthesized
gacatccagatgacccagtctccatcctccctgtctgcatctgtaggagacagagtcaccatcacttgccaggccagtcagagcatttacagctacttaaactggtatcagcagaaaccagggaaagcccctaagctcctgatctatggtgcatccaatctggcatctggggtcccatcaaggttcagtggcagtggatctgggacagatttcactctcaccatcagcagtctgcaacctgaagattttgcaacttactactgtcaaagcagttggttgagtggtgctgttggtaatgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 56 - protein, 217 aa, Artificial Sequence
  synthesized
DIQMTQSPSSLSASVGDRVTITCQASQSIYSYLNWYQQKPGKAPKLLIYGASNLASGVPSRFSGSGSGTDFTLTISSLQPEDFATYYCQSSWLSGAVGNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 2094 bp, Artificial Sequence
  synthesized
caggagcaggtgaaggagaccgggggaggcttggtacagcctggggggtccctgagactctcctgtgcagcctctggattcaccatcagcagctatggagtgagctgggtccgccaggctccagggaaggggctggagtgggtcgcattgatttttcccgggattggtttcaaagactacgcgagctgggtgaatggccggttcaccctctccagcgacaacgcccagaacactgtggaactgcaaatgaacagcctgagagccgaggacacggccgtatattactgtgcgagagatttggacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 58 - protein, 698 aa, Artificial Sequence
  synthesized
QEQVKETGGGLVQPGGSLRLSCAASGFTISSYGVSWVRQAPGKGLEWVALIFPGIGFKDYASWVNGRFTLSSDNAQNTVELQMNSLRAEDTAVYYCARDLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 59 - DNA, 648 bp, Artificial Sequence
  synthesized
gacatccagatgacccagtctccatcctccctgtctgcatctgtaggagacagagtcaccatcacttgccagtccagtccgagtgtttatagtaactacttatcctggtatcagcagaaaccagggaaagttcctaagctcctgatctattatgcatccactctggcatctggggtcccatctcggttcagtggcagtggatctgggacagatttcactctcaccatcagcagcctgcagcctgaagatgttgcaacttattactgtgcaggcggttatagtagtagtactcgtgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 60 - protein, 216 aa, Artificial Sequence
  synthesized
DIQMTQSPSSLSASVGDRVTITCQSSPSVYSNYLSWYQQKPGKVPKLLIYYASTLASGVPSRFSGSGSGTDFTLTISSLQPEDVATYYCAGGYSSSTRAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 2121 bp, Artificial Sequence
  synthesized
gaggtgcagctgttggagtctgggggaggcttggtacagcctggggggtccctgagactctcctgtgcagcctctggattcacctttagcagcagccactggatatgctgggtccgccaggctccagggaaggggctggagtggatcgcatgcatttatactggtagtattgatgtcttttactacgcgagctgggcgaaaggccggttcaccatctccagagacaattccaagaacacgctgtatctgcaaatgaacagcctgagagccgaggacacggccgtatattactgtgcgagagccgctaatactgatactacctactttaacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 62 - protein, 707 aa, Artificial Sequence
  synthesized
EVQLLESGGGLVQPGGSLRLSCAASGFTFSSSHWICWVRQAPGKGLEWIACIYTGSIDVFYYASWAKGRFTISRDNSKNTLYLQMNSLRAEDTAVYYCARAANTDTTYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 63 - DNA, 654 bp, Artificial Sequence
  synthesized
gcctatgatatgacccagtctccatcctccctgtctgcatctgtaggagacagagtcaccatcaattgccaggccagtcagagcattaacaaccaactatcctggtatcagcagaaaccagggaaagttcctaagctcctgatctatggtgcatccactctggcatctggggtcccatctcggttcaccggcagtggatctgggacagatttcactctcaccatcagcagcctgcagcctgaagatgttgcaacttattactgtcatgttcattattgcagtggtggtagttgtttttgggctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 64 - protein, 218 aa, Artificial Sequence
  synthesized
AYDMTQSPSSLSASVGDRVTINCQASQSINNQLSWYQQKPGKVPKLLIYGASTLASGVPSRFTGSGSGTDFTLTISSLQPEDVATYYCHVHYCSGGSCFWAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - DNA, 57 bp, Artificial Sequence
  synthesized
atgaaacacctgtggttcttcctcctcctggtggcagctcccagatgggtcctgtcc

SEQ ID NO: 66 - protein, 19 aa, Artificial Sequence
  synthesized
MKHLWFFLLLVAAPRWVLS

SEQ ID NO: 67 - DNA, 57 bp, Artificial Sequence
  synthesized
atggctgtcttggggctgctcttctgcctggtgacattcccaagctgtgtcctatcc

SEQ ID NO: 68 - protein, 19 aa, Artificial Sequence
  synthesized
MAVLGLLFCLVTFPSCVLS

SEQ ID NO: 69 - DNA, 60 bp, Artificial Sequence
  synthesized
atgagggcccctgctcagtttcttggcttcttgcttttctggattccagcctccagaagt

SEQ ID NO: 70 - protein, 20 aa, Artificial Sequence
  synthesized
MRAPAQFLGFLLFWIPASRS

SEQ ID NO: 71 - DNA, 738 bp, Artificial Sequence
  synthesized
caggtgcaattggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcgagcggtggaggcggatctggcggaggtggttccggcggtggcggctccggtggaggcggttctgaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 72 - protein, 246 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 73 - DNA, 732 bp, Artificial Sequence
  synthesized
gaggtgcagctggtggagtctgggggaggcttggtccagcctggggggtccctgagactctcctgtacagcctctggattctccctcagtagctatgcaatgagctgggtccgccaggctccagggaaggggctggagtacatcggctacattggtgatactactggcatagcctacgcgagctgggcgaatggcagattcaccatctccaaagacaataccaagaacacggtggatcttcaaatgaacagcctgagagccgaggacacggctgtgtattactgtgcgagaggctggtcctacttagacatctggggccaagggaccctggtcaccgtctcgagcggtggaggcggatctggcggaggtggttccggcggtggcggctccggtggaggcggctctgcccttgtgatgacccagtctccatcctccctgtctgcatctgtaggagacagagtcaccatcacttgccaggccagtcagaacatttacagcaatttagcctggtatcagcagaaaccagggaaagttcctaagctcctgatctatcaggcctccactctggcatctggggtcccatctcggttcagtggcagtggatatgggacagatttcactctcaccatcagcagcctgcagcctgaagatgttgcaacttattactgtcaaggcggttattatagtgctgcccttaatactttcggcggagggaccaaggtggagatcaaa

SEQ ID NO: 74 - protein, 244 aa, Artificial Sequence
  synthesized
EVQLVESGGGLVQPGGSLRLSCTASGFSLSSYAMSWVRQAPGKGLEYIGYIGDTTGIAYASWANGRFTISKDNTKNTVDLQMNSLRAEDTAVYYCARGWSYLDIWGQGTLVTVSSGGGGSGGGGSGGGGSGGGGSALVMTQSPSSLSASVGDRVTITCQASQNIYSNLAWYQQKPGKVPKLLIYQASTLASGVPSRFSGSGYGTDFTLTISSLQPEDVATYYCQGGYYSAALNTFGGGTKVEIK

SEQ ID NO: 75 - DNA, 750 bp, Artificial Sequence
  synthesized
gaggtgcagctggtggagtctgggggaggcttggtccagcctggggggtccctgagactctcctgtgcagcctctggattcgacttcagtagcggctactggatatgctgggtccgccaggctccagggaaggggctggagttgatcgcatgcatttatgctggtactagtggtagtacttcctacgcgagctgggcgagaggcagattcaccatctccgaaacctccaagaacacggtgactcttcaaatgaacagcctgagagccgaggactcggctgtgtattactgtgcgagaaatctttacacttacaatagcttgtggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccagtccagtcagagtgtttatgataacaactggttagcctggtatcagcagaaaccagggaaagcccctaagctcctgatctatacagtatccactctggcatctggggtcccatcaaggttcagcggcagtggatctgggacagaattcactctcaccatcagcagcctgcagcctgatgattttgcaacttattactgccaaggcacttattatagtagtggttggaactttgctttcggcggagggaccaaggtggagatcaaa

SEQ ID NO: 76 - protein, 250 aa, Artificial Sequence
  synthesized
EVQLVESGGGLVQPGGSLRLSCAASGFDFSSGYWICWVRQAPGKGLELIACIYAGTSGSTSYASWARGRFTISETSKNTVTLQMNSLRAEDSAVYYCARNLYTYNSLWGQGTLVTVSSGGGGSGGGGSGGGGSGGGGSDIQMTQSPSTLSASVGDRVTITCQSSQSVYDNNWLAWYQQKPGKAPKLLIYTVSTLASGVPSRFSGSGSGTEFTLTISSLQPDDFATYYCQGTYYSSGWNFAFGGGTKVEIK

SEQ ID NO: 77 - DNA, 729 bp, Artificial Sequence
  synthesized
caggagcagctgttggagtctgggggaggcttggtacagcctggggggtccctgagactctcctgtacagcctctggattctcctttagcagctactggatgtgctgggtccgccaggctccagggaaggggctggagtggatcggatgcattacgactggtagtggtagcacttactacgcgagctgggcgaagcgccggttcaccatctccaaagacaattccaagaacacggtgactctgcaaatgaacagcctgagagccgaggacacggccgtatattactgtacgagagcatttgacttgtggggccagggaaccctggtcaccgtctcgagcggtggaggcggatctggcggaggtggttccggcggtggcggctccggtggaggcggctctgacatccagatgacccagtctccatcctccctgtctgcatctgtaggagacagagtcaccatcacttgccaggccagtcagagcatttacagctacttaaactggtatcagcagaaaccagggaaagcccctaagctcctgatctatggtgcatccaatctggcatctggggtcccatcaaggttcagtggcagtggatctgggacagatttcactctcaccatcagcagtctgcaacctgaagattttgcaacttactactgtcaaagcagttggttgagtggtgctgttggtaatgctttcggcggagggaccaaggtggagatcaaa

SEQ ID NO: 78 - protein, 243 aa, Artificial Sequence
  synthesized
QEQLLESGGGLVQPGGSLRLSCTASGFSFSSYWMCWVRQAPGKGLEWIGCITTGSGSTYYASWAKRRFTISKDNSKNTVTLQMNSLRAEDTAVYYCTRAFDLWGQGTLVTVSSGGGGSGGGGSGGGGSGGGGSDIQMTQSPSSLSASVGDRVTITCQASQSIYSYLNWYQQKPGKAPKLLIYGASNLASGVPSRFSGSGSGTDFTLTISSLQPEDFATYYCQSSWLSGAVGNAFGGGTKVEIK

SEQ ID NO: 79 - DNA, 759 bp, Artificial Sequence
  synthesized
gaggtgcagctgttggagtctgggggaggcttggtacagcctggggggtccctgagactctcctgtgcagcctctggattcacctttagcagcagccactggatatgctgggtccgccaggctccagggaaggggctggagtggatcgcatgcatttatactggtagtattgatgtcttttactacgcgagctgggcgaaaggccggttcaccatctccagagacaattccaagaacacgctgtatctgcaaatgaacagcctgagagccgaggacacggccgtatattactgtgcgagagccgctaatactgatactacctactttaacttgtggggccagggaaccctggtcaccgtctcgagcggtggaggcggatctggcggaggtggttccggcggtggcggctccggtggaggcggctctgcctatgatatgacccagtctccatcctccctgtctgcatctgtaggagacagagtcaccatcaattgccaggccagtcagagcattaacaaccaactatcctggtatcagcagaaaccagggaaagttcctaagctcctgatctatggtgcatccactctggcatctggggtcccatctcggttcaccggcagtggatctgggacagatttcactctcaccatcagcagcctgcagcctgaagatgttgcaacttattactgtcatgttcattattgcagtggtggtagttgtttttgggctttcggcggagggaccaaggtggagatcaaa

SEQ ID NO: 80 - protein, 253 aa, Artificial Sequence
  synthesized
EVQLLESGGGLVQPGGSLRLSCAASGFTFSSSHWICWVRQAPGKGLEWIACIYTGSIDVFYYASWAKGRFTISRDNSKNTLYLQMNSLRAEDTAVYYCARAANTDTTYFNLWGQGTLVTVSSGGGGSGGGGSGGGGSGGGGSAYDMTQSPSSLSASVGDRVTINCQASQSINNQLSWYQQKPGKVPKLLIYGASTLASGVPSRFTGSGSGTDFTLTISSLQPEDVATYYCHVHYCSGGSCFWAFGGGTKVEIK

SEQ ID NO: 81 - DNA, 735 bp, Artificial Sequence
  synthesized
cagtcggtggaggagtctgggggaggcttggtccagcctggggggtccctgagactctcctgtaccgcctctggaatcgaccttaatacctacgacatgatctgggtccgccaggctccaggcaaggggctagagtgggttggaatcattacttatagtggtagtagatactacgcgaactgggcgaaaggccgattcaccatctccaaagacaataccaagaacacggtgtatctgcaaatgaacagcctgagagctgaggacacggctgtgtattactgtgcgagagattatatgagtggttcccacttgtggggccagggaaccctggtcaccgtctcttccggtggaggcggttcaggcggaggtggaagtggtggtggcggctctggaggcggcggatctgcctatgatatgacccagtctccatcttccgtgtctgcatctgtaggagacagagtcaccatcaagtgtcaggccagtgaggacatttatagcttcttggcctggtatcagcagaaaccagggaaagcccctaagctcctgatccattctgcatcctctctggcatctggggtcccatcaaggttcagcggcagtggatctgggacagatttcactctcaccatcagcagcctgcagcctgaagattttgcaacttactattgtcaacagggttatggtaaaaataatgttgataatgctttcggcggagggaccaaggtggagatcaaa

SEQ ID NO: 82 - protein, 245 aa, Artificial Sequence
  synthesized
QSVEESGGGLVQPGGSLRLSCTASGIDLNTYDMIWVRQAPGKGLEWVGIITYSGSRYYANWAKGRFTISKDNTKNTVYLQMNSLRAEDTAVYYCARDYMSGSHLWGQGTLVTVSSGGGGSGGGGSGGGGSGGGGSAYDMTQSPSSVSASVGDRVTIKCQASEDIYSFLAWYQQKPGKAPKLLIHSASSLASGVPSRFSGSGSGTDFTLTISSLQPEDFATYYCQQGYGKNNVDNAFGGGTKVEIK

SEQ ID NO: 83 - DNA, 528 bp, Artificial Sequence
  synthesized
aaagacaatacctcgaccacggtggatctgcaaatgaacagcctgagagctgaggacacggctgtgtattactgtgcgagagattttattagtggttcccacttgtggggccagggaaccctggtcaccgtctcgagcggtggaggcggatctggcggaggtggttccggcggtggcggctccggtggaggcggttctgcctatgatatgacccagtctccatcttccgtgtctgcatctgtaggagacagagtcaccatcaattgtcaggccagtgagagcattagtagcttcttatcctggtatcagcagaaaccagggaaagcccctaagctcctgatcttttctgcatccactctggcatctggggtcccatcaaggttcagcggcagtggatctgggacagatttcactctcaccatcagcagcctgcagcctgaagattttgcaacttactattgtcaacagggttatagtaaaagtaatgtggataatgctttcggcggagggaccaaggtggagatcaaa

SEQ ID NO: 84 - protein, 246 aa, Artificial Sequence
  synthesized
QVQLVESGGGLVQPGGSLRLSCTTSGIDLSTYDMIWVRQAPGKGLEWVGIISYVGNTYYASWAKGRFTLSKDNTSTTVDLQMNSLRAEDTAVYYCARDFISGSHLWGQGTLVTVSSGGGGSGGGGSGGGGSGGGGSAYDMTQSPSSVSASVGDRVTINCQASESISSFLSWYQQKPGKAPKLLIFSASTLASGVPSRFSGSGSGTDFTLTISSLQPEDFATYYCQQGYSKSNVDNAFGGGTKVEIK

SEQ ID NO: 85 - DNA, 756 bp, Artificial Sequence
  synthesized
gaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaa

SEQ ID NO: 86 - protein, 252 aa, Artificial Sequence
  synthesized
EVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIK

SEQ ID NO: 87 - DNA, 750 bp, Artificial Sequence
  synthesized
gaggtgcagctggtggagtctgggggaggcttggtccagcctggggggtccctgagactctcctgtgcagcctctggattctccctcagtagctactggatgagctgggtccgccaggctccagggaaggggctggagtggatcggagtcattgatactaatgtttatatatactacgcgaactgggcaaaaggcagattcaccatctccagagacaattccaagaacacgctgtatcttcaaatgaacagcctgagagccgaggacacggctgtgtattactgtgcgagatatgtgggtaataatgatgattatattaacttgtggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccagtccagtcagagtgtttataatggctactggttatcctggtatcagcagaaaccagggaaagcccctaagctcctgatctatggtgcatccactctggcatctggggtcccatcaaggttcagcggcagtggatctgggacagaattcactctcaccatcagcagcctgcagcctgatgattttgcaacttattactgcctaggcagttatactagtagtactgagaactctttcggcggagggaccaaggtggagatcaaa

SEQ ID NO: 88 - protein, 250 aa, Artificial Sequence
  synthesized
EVQLVESGGGLVQPGGSLRLSCAASGFSLSSYWMSWVRQAPGKGLEWIGVIDTNVYIYYANWAKGRFTISRDNSKNTLYLQMNSLRAEDTAVYYCARYVGNNDDYINLWGQGTLVTVSSGGGGSGGGGSGGGGSGGGGSDIQMTQSPSTLSASVGDRVTITCQSSQSVYNGYWLSWYQQKPGKAPKLLIYGASTLASGVPSRFSGSGSGTEFTLTISSLQPDDFATYYCLGSYTSSTENSFGGGTKVEIK

SEQ ID NO: 89 - DNA, 741 bp, Artificial Sequence
  synthesized
gaggtgcagctggtggagtctgggggaggcttggtccagcctggggggtccctgagactctcctgtacagtgtctggaatcgacctcagtgtcatcaatatgggctgggtccgccaggctccagggaaggggctggagtggatcggaaccattacttatgttggtaacacatattacgcgagctgggcgaaaggcagattcaccatctccaaaacctcgaccacggtggatcttaaaatcaccagtccgacaaccgaggacacggctgtgtattactgtgcgagagaatctggtactatttattacagttactttaacttgtggggccaagggaccctggtcaccgtctcgagcggtggaggcggatctggcggaggtggttccggcggtggcggctccggtggaggcggctctgcattcgaattgacccagtctccatcctccctgtctgcatctgtaggagacagagtcaccatcaagtgccaggccagtgaaagcattagcaactacttatcctggtatcagcagattccagggaaagttcctaagctcctgatctattatgcatccaatctggcatctggggtcccatctcggttcagtggcagtggatctgggacagatttcactctcaccatcagcagcctgcagcctgaagatgttgcaacttattactgtcaaagctattatggtggtggtagtgcctatactttcggcggagggaccaaggtggagatcaaa

SEQ ID NO: 90 - protein, 247 aa, Artificial Sequence
  synthesized
EVQLVESGGGLVQPGGSLRLSCTVSGIDLSVINMGWVRQAPGKGLEWIGTITYVGNTYYASWAKGRFTISKTSTTVDLKITSPTTEDTAVYYCARESGTIYYSYFNLWGQGTLVTVSSGGGGSGGGGSGGGGSGGGGSAFELTQSPSSLSASVGDRVTIKCQASESISNYLSWYQQIPGKVPKLLIYYASNLASGVPSRFSGSGSGTDFTLTISSLQPEDVATYYCQSYYGGGSAYTFGGGTKVEIK

SEQ ID NO: 91 - DNA, 354 bp, Artificial Sequence
  synthesized
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctca

SEQ ID NO: 92 - protein, 118 aa, Artificial Sequence
  synthesized
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 93 - DNA, 324 bp, Artificial Sequence
  synthesized
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 94 - protein, 108 aa, Artificial Sequence
  synthesized
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 95 - DNA, 345 bp, Artificial Sequence
  synthesized
gaggtgcagctggtggagtctgggggaggcttggtccagcctggggggtccctgagactctcctgtacagcctctggattctccctcagtagctatgcaatgagctgggtccgccaggctccagggaaggggctggagtacatcggctacattggtgatactactggcatagcctacgcgagctgggcgaatggcagattcaccatctccaaagacaataccaagaacacggtggatcttcaaatgaacagcctgagagccgaggacacggctgtgtattactgtgcgagaggctggtcctacttagacatctggggccaagggaccctggtcaccgtctcgagc

SEQ ID NO: 96 - protein, 115 aa, Artificial Sequence
  synthesized
EVQLVESGGGLVQPGGSLRLSCTASGFSLSSYAMSWVRQAPGKGLEYIGYIGDTTGIAYASWANGRFTISKDNTKNTVDLQMNSLRAEDTAVYYCARGWSYLDIWGQGTLVTVSS

SEQ ID NO: 97 - DNA, 327 bp, Artificial Sequence
  synthesized
gcccttgtgatgacccagtctccatcctccctgtctgcatctgtaggagacagagtcaccatcacttgccaggccagtcagaacatttacagcaatttagcctggtatcagcagaaaccagggaaagttcctaagctcctgatctatcaggcctccactctggcatctggggtcccatctcggttcagtggcagtggatatgggacagatttcactctcaccatcagcagcctgcagcctgaagatgttgcaacttattactgtcaaggcggttattatagtgctgcccttaatactttcggcggagggaccaaggtggagatcaaa

SEQ ID NO: 98 - protein, 109 aa, Artificial Sequence
  synthesized
ALVMTQSPSSLSASVGDRVTITCQASQNIYSNLAWYQQKPGKVPKLLIYQASTLASGVPSRFSGSGYGTDFTLTISSLQPEDVATYYCQGGYYSAALNTFGGGTKVEIK

SEQ ID NO: 99 - DNA, 354 bp, Artificial Sequence
  synthesized
gaggtgcagctggtggagtctgggggaggcttggtccagcctggggggtccctgagactctcctgtgcagcctctggattcgacttcagtagcggctactggatatgctgggtccgccaggctccagggaaggggctggagttgatcgcatgcatttatgctggtactagtggtagtacttcctacgcgagctgggcgagaggcagattcaccatctccgaaacctccaagaacacggtgactcttcaaatgaacagcctgagagccgaggactcggctgtgtattactgtgcgagaaatctttacacttacaatagcttgtggggccagggaaccctggtcaccgtctcgagc

SEQ ID NO: 100 - protein, 118 aa, Artificial Sequence
  synthesized
EVQLVESGGGLVQPGGSLRLSCAASGFDFSSGYWICWVRQAPGKGLELIACIYAGTSGSTSYASWARGRFTISETSKNTVTLQMNSLRAEDSAVYYCARNLYTYNSLWGQGTLVTVSS

SEQ ID NO: 101 - DNA, 336 bp, Artificial Sequence
  synthesized
gacatccagatgacccagtctccttccaccctgtctgcatctgtaggagacagagtcaccatcacttgccagtccagtcagagtgtttatgataacaactggttagcctggtatcagcagaaaccagggaaagcccctaagctcctgatctatacagtatccactctggcatctggggtcccatcaaggttcagcggcagtggatctgggacagaattcactctcaccatcagcagcctgcagcctgatgattttgcaacttattactgccaaggcacttattatagtagtggttggaactttgctttcggcggagggaccaaggtggagatcaaa

SEQ ID NO: 102 - protein, 112 aa, Artificial Sequence
  synthesized
DIQMTQSPSTLSASVGDRVTITCQSSQSVYDNNWLAWYQQKPGKAPKLLIYTVSTLASGVPSRFSGSGSGTEFTLTISSLQPDDFATYYCQGTYYSSGWNFAFGGGTKVEIK

SEQ ID NO: 103 - DNA, 339 bp, Artificial Sequence
  synthesized
caggagcagctgttggagtctgggggaggcttggtacagcctggggggtccctgagactctcctgtacagcctctggattctcctttagcagctactggatgtgctgggtccgccaggctccagggaaggggctggagtggatcggatgcattacgactggtagtggtagcacttactacgcgagctgggcgaagcgccggttcaccatctccaaagacaattccaagaacacggtgactctgcaaatgaacagcctgagagccgaggacacggccgtatattactgtacgagagcatttgacttgtggggccagggaaccctggtcaccgtctcgagc

SEQ ID NO: 104 - protein, 113 aa, Artificial Sequence
  synthesized
QEQLLESGGGLVQPGGSLRLSCTASGFSFSSYWMCWVRQAPGKGLEWIGCITTGSGSTYYASWAKRRFTISKDNSKNTVTLQMNSLRAEDTAVYYCTRAFDLWGQGTLVTVSS

SEQ ID NO: 105 - DNA, 330 bp, Artificial Sequence
  synthesized
gacatccagatgacccagtctccatcctccctgtctgcatctgtaggagacagagtcaccatcacttgccaggccagtcagagcatttacagctacttaaactggtatcagcagaaaccagggaaagcccctaagctcctgatctatggtgcatccaatctggcatctggggtcccatcaaggttcagtggcagtggatctgggacagatttcactctcaccatcagcagtctgcaacctgaagattttgcaacttactactgtcaaagcagttggttgagtggtgctgttggtaatgctttcggcggagggaccaaggtggagatcaaa

SEQ ID NO: 106 - protein, 110 aa, Artificial Sequence
  synthesized
DIQMTQSPSSLSASVGDRVTITCQASQSIYSYLNWYQQKPGKAPKLLIYGASNLASGVPSRFSGSGSGTDFTLTISSLQPEDFATYYCQSSWLSGAVGNAFGGGTKVEIK

SEQ ID NO: 107 - DNA, 366 bp, Artificial Sequence
  synthesized
gaggtgcagctgttggagtctgggggaggcttggtacagcctggggggtccctgagactctcctgtgcagcctctggattcacctttagcagcagccactggatatgctgggtccgccaggctccagggaaggggctggagtggatcgcatgcatttatactggtagtattgatgtcttttactacgcgagctgggcgaaaggccggttcaccatctccagagacaattccaagaacacgctgtatctgcaaatgaacagcctgagagccgaggacacggccgtatattactgtgcgagagccgctaatactgatactacctactttaacttgtggggccagggaaccctggtcaccgtctcgagc

SEQ ID NO: 108 - protein, 122 aa, Artificial Sequence
  synthesized
EVQLLESGGGLVQPGGSLRLSCAASGFTFSSSHWICWVRQAPGKGLEWIACIYTGSIDVFYYASWAKGRFTISRDNSKNTLYLQMNSLRAEDTAVYYCARAANTDTTYFNLWGQGTLVTVSS

SEQ ID NO: 109 - DNA, 333 bp, Artificial Sequence
  synthesized
gcctatgatatgacccagtctccatcctccctgtctgcatctgtaggagacagagtcaccatcaattgccaggccagtcagagcattaacaaccaactatcctggtatcagcagaaaccagggaaagttcctaagctcctgatctatggtgcatccactctggcatctggggtcccatctcggttcaccggcagtggatctgggacagatttcactctcaccatcagcagcctgcagcctgaagatgttgcaacttattactgtcatgttcattattgcagtggtggtagttgtttttgggctttcggcggagggaccaaggtggagatcaaa

SEQ ID NO: 110 - protein, 111 aa, Artificial Sequence
  synthesized
AYDMTQSPSSLSASVGDRVTINCQASQSINNQLSWYQQKPGKVPKLLIYGASTLASGVPSRFTGSGSGTDFTLTISSLQPEDVATYYCHVHYCSGGSCFWAFGGGTKVEIK

SEQ ID NO: 111 - DNA, 345 bp, Artificial Sequence
  synthesized
cagtcggtggaggagtctgggggaggcttggtccagcctggggggtccctgagactctcctgtaccgcctctggaatcgaccttaatacctacgacatgatctgggtccgccaggctccaggcaaggggctagagtgggttggaatcattacttatagtggtagtagatactacgcgaactgggcgaaaggccgattcaccatctccaaagacaataccaagaacacggtgtatctgcaaatgaacagcctgagagctgaggacacggctgtgtattactgtgcgagagattatatgagtggttcccacttgtggggccagggaaccctggtcaccgtctcttcc

SEQ ID NO: 112 - protein, 115 aa, Artificial Sequence
  synthesized
QSVEESGGGLVQPGGSLRLSCTASGIDLNTYDMIWVRQAPGKGLEWVGIITYSGSRYYANWAKGRFTISKDNTKNTVYLQMNSLRAEDTAVYYCARDYMSGSHLWGQGTLVTVSS

SEQ ID NO: 113 - DNA, 330 bp, Artificial Sequence
  synthesized
gcctatgatatgacccagtctccatcttccgtgtctgcatctgtaggagacagagtcaccatcaagtgtcaggccagtgaggacatttatagcttcttggcctggtatcagcagaaaccagggaaagcccctaagctcctgatccattctgcatcctctctggcatctggggtcccatcaaggttcagcggcagtggatctgggacagatttcactctcaccatcagcagcctgcagcctgaagattttgcaacttactattgtcaacagggttatggtaaaaataatgttgataatgctttcggcggagggaccaaggtggagatcaaa

SEQ ID NO: 114 - protein, 110 aa, Artificial Sequence
  synthesized
AYDMTQSPSSVSASVGDRVTIKCQASEDIYSFLAWYQQKPGKAPKLLIHSASSLASGVPSRFSGSGSGTDFTLTISSLQPEDFATYYCQQGYGKNNVDNAFGGGTKVEIK

SEQ ID NO: 115 - DNA, 348 bp, Artificial Sequence
  synthesized
caggtgcagctggtggagtctgggggaggcctggtccagcctgggggctccctgagactctcctgtacaacttctggaatcgaccttagtacctacgacatgatctgggtccgccaggctccaggcaaggggctagagtgggtgggaatcattagttatgttggtaacacatactacgcgagctgggcgaaaggccgattcaccctctccaaagacaatacctcgaccacggtggatctgcaaatgaacagcctgagagctgaggacacggctgtgtattactgtgcgagagattttattagtggttcccacttgtggggccagggaaccctggtcaccgtctcgagc

SEQ ID NO: 116 - protein, 116 aa, Artificial Sequence
  synthesized
QVQLVESGGGLVQPGGSLRLSCTTSGIDLSTYDMIWVRQAPGKGLEWVGIISYVGNTYYASWAKGRFTLSKDNTSTTVDLQMNSLRAEDTAVYYCARDFISGSHLWGQGTLVTVSS

SEQ ID NO: 117 - DNA, 330 bp, Artificial Sequence
  synthesized
gcctatgatatgacccagtctccatcttccgtgtctgcatctgtaggagacagagtcaccatcaattgtcaggccagtgagagcattagtagcttcttatcctggtatcagcagaaaccagggaaagcccctaagctcctgatcttttctgcatccactctggcatctggggtcccatcaaggttcagcggcagtggatctgggacagatttcactctcaccatcagcagcctgcagcctgaagattttgcaacttactattgtcaacagggttatagtaaaagtaatgtggataatgctttcggcggagggaccaaggtggagatcaaa

SEQ ID NO: 118 - protein, 110 aa, Artificial Sequence
  synthesized
AYDMTQSPSSVSASVGDRVTINCQASESISSFLSWYQQKPGKAPKLLIFSASTLASGVPSRFSGSGSGTDFTLTISSLQPEDFATYYCQQGYSKSNVDNAFGGGTKVEIK

SEQ ID NO: 119 - DNA, 366 bp, Artificial Sequence
  synthesized
gaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagc

SEQ ID NO: 120 - protein, 122 aa, Artificial Sequence
  synthesized
EVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSS

SEQ ID NO: 121 - DNA, 330 bp, Artificial Sequence
  synthesized
gacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaa

SEQ ID NO: 122 - protein, 110 aa, Artificial Sequence
  synthesized
DIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIK

SEQ ID NO: 123 - DNA, 357 bp, Artificial Sequence
  synthesized
gaggtgcagctggtggagtctgggggaggcttggtccagcctggggggtccctgagactctcctgtgcagcctctggattctccctcagtagctactggatgagctgggtccgccaggctccagggaaggggctggagtggatcggagtcattgatactaatgtttatatatactacgcgaactgggcaaaaggcagattcaccatctccagagacaattccaagaacacgctgtatcttcaaatgaacagcctgagagccgaggacacggctgtgtattactgtgcgagatatgtgggtaataatgatgattatattaacttgtggggccagggaaccctggtcaccgtctcgagc

SEQ ID NO: 124 - protein, 119 aa, Artificial Sequence
  synthesized
EVQLVESGGGLVQPGGSLRLSCAASGFSLSSYWMSWVRQAPGKGLEWIGVIDTNVYIYYANWAKGRFTISRDNSKNTLYLQMNSLRAEDTAVYYCARYVGNNDDYINLWGQGTLVTVSS

SEQ ID NO: 125 - DNA, 333 bp, Artificial Sequence
  synthesized
gacatccagatgacccagtctccttccaccctgtctgcatctgtaggagacagagtcaccatcacttgccagtccagtcagagtgtttataatggctactggttatcctggtatcagcagaaaccagggaaagcccctaagctcctgatctatggtgcatccactctggcatctggggtcccatcaaggttcagcggcagtggatctgggacagaattcactctcaccatcagcagcctgcagcctgatgattttgcaacttattactgcctaggcagttatactagtagtactgagaactctttcggcggagggaccaaggtggagatcaaa

SEQ ID NO: 126 - protein, 111 aa, Artificial Sequence
  synthesized
DIQMTQSPSTLSASVGDRVTITCQSSQSVYNGYWLSWYQQKPGKAPKLLIYGASTLASGVPSRFSGSGSGTEFTLTISSLQPDDFATYYCLGSYTSSTENSFGGGTKVEIK

SEQ ID NO: 127 - DNA, 354 bp, Artificial Sequence
  synthesized
gaggtgcagctggtggagtctgggggaggcttggtccagcctggggggtccctgagactctcctgtacagtgtctggaatcgacctcagtgtcatcaatatgggctgggtccgccaggctccagggaaggggctggagtggatcggaaccattacttatgttggtaacacatattacgcgagctgggcgaaaggcagattcaccatctccaaaacctcgaccacggtggatcttaaaatcaccagtccgacaaccgaggacacggctgtgtattactgtgcgagagaatctggtactatttattacagttactttaacttgtggggccaagggaccctggtcaccgtctcgagc

SEQ ID NO: 128 - protein, 118 aa, Artificial Sequence
  synthesized
EVQLVESGGGLVQPGGSLRLSCTVSGIDLSVINMGWVRQAPGKGLEWIGTITYVGNTYYASWAKGRFTISKTSTTVDLKITSPTTEDTAVYYCARESGTIYYSYFNLWGQGTLVTVSS

SEQ ID NO: 129 - DNA, 327 bp, Artificial Sequence
  synthesized
gcattcgaattgacccagtctccatcctccctgtctgcatctgtaggagacagagtcaccatcaagtgccaggccagtgaaagcattagcaactacttatcctggtatcagcagattccagggaaagttcctaagctcctgatctattatgcatccaatctggcatctggggtcccatctcggttcagtggcagtggatctgggacagatttcactctcaccatcagcagcctgcagcctgaagatgttgcaacttattactgtcaaagctattatggtggtggtagtgcctatactttcggcggagggaccaaggtggagatcaaa

SEQ ID NO: 130 - protein, 109 aa, Artificial Sequence
  synthesized
AFELTQSPSSLSASVGDRVTIKCQASESISNYLSWYQQIPGKVPKLLIYYASNLASGVPSRFSGSGSGTDFTLTISSLQPEDVATYYCQSYYGGGSAYTFGGGTKVEIK

SEQ ID NO: 131 - DNA, 339 bp, Artificial Sequence
  synthesized
caggagcaggtgaaggagaccgggggaggcttggtacagcctggggggtccctgagactctcctgtgcagcctctggattcaccatcagcagctatggagtgagctgggtccgccaggctccagggaaggggctggagtgggtcgcattgatttttcccgggattggtttcaaagactacgcgagctgggtgaatggccggttcaccctctccagcgacaacgcccagaacactgtggaactgcaaatgaacagcctgagagccgaggacacggccgtatattactgtgcgagagatttggacttgtggggccagggaaccctggtcaccgtctcgagc

SEQ ID NO: 132 - protein, 113 aa, Artificial Sequence
  synthesized
QEQVKETGGGLVQPGGSLRLSCAASGFTISSYGVSWVRQAPGKGLEWVALIFPGIGFKDYASWVNGRFTLSSDNAQNTVELQMNSLRAEDTAVYYCARDLDLWGQGTLVTVSS

SEQ ID NO: 133 - DNA, 327 bp, Artificial Sequence
  synthesized
gacatccagatgacccagtctccatcctccctgtctgcatctgtaggagacagagtcaccatcacttgccagtccagtccgagtgtttatagtaactacttatcctggtatcagcagaaaccagggaaagttcctaagctcctgatctattatgcatccactctggcatctggggtcccatctcggttcagtggcagtggatctgggacagatttcactctcaccatcagcagcctgcagcctgaagatgttgcaacttattactgtgcaggcggttatagtagtagtactcgtgctttcggcggagggaccaaggtggagatcaaa

SEQ ID NO: 134 - protein, 109 aa, Artificial Sequence
  synthesized
DIQMTQSPSSLSASVGDRVTITCQSSPSVYSNYLSWYQQKPGKVPKLLIYYASTLASGVPSRFSGSGSGTDFTLTISSLQPEDVATYYCAGGYSSSTRAFGGGTKVEIK

SEQ ID NO: 135 - DNA, 987 bp, Artificial Sequence
  synthesized
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 136 - protein, 329 aa, Artificial Sequence
  synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - DNA, 987 bp, Artificial Sequence
  synthesized
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 138 - protein, 329 aa, Artificial Sequence
  synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - DNA, 726 bp, Artificial Sequence
  Synthesized
caggagcaggtgaaggagaccgggggaggcttggtacagcctggggggtccctgagactctcctgtgcagcctctggattcaccatcagcagctatggagtgagctgggtccgccaggctccagggaaggggctggagtgggtcgcattgatttttcccgggattggtttcaaagactacgcgagctgggtgaatggccggttcaccctctccagcgacaacgcccagaacactgtggaactgcaaatgaacagcctgagagccgaggacacggccgtatattactgtgcgagagatttggacttgtggggccagggaaccctggtcaccgtctcgagcggtggaggcggatctggcggaggtggttccggcggtggcggctccggtggaggcggctctgacatccagatgacccagtctccatcctccctgtctgcatctgtaggagacagagtcaccatcacttgccagtccagtccgagtgtttatagtaactacttatcctggtatcagcagaaaccagggaaagttcctaagctcctgatctattatgcatccactctggcatctggggtcccatctcggttcagtggcagtggatctgggacagatttcactctcaccatcagcagcctgcagcctgaagatgttgcaacttattactgtgcaggcggttatagtagtagtactcgtgctttcggcggagggaccaaggtggagatcaaa

SEQ ID NO: 140 - protein, 242 aa, Artificial Sequence
  Synthesized
QEQVKETGGGLVQPGGSLRLSCAASGFTISSYGVSWVRQAPGKGLEWVALIFPGIGFKDYASWVNGRFTLSSDNAQNTVELQMNSLRAEDTAVYYCARDLDLWGQGTLVTVSSGGGGSGGGGSGGGGSGGGGSDIQMTQSPSSLSASVGDRVTITCQSSPSVYSNYLSWYQQKPGKVPKLLIYYASTLASGVPSRFSGSGSGTDFTLTISSLQPEDVATYYCAGGYSSSTRAFGGGTKVEIK

SEQ ID NO: 141 - protein, 6 aa, Artificial Sequence
  Synthesized
SSYTMH

SEQ ID NO: 142 - protein, 17 aa, Artificial Sequence
  Synthesized
FISYDGNNKYYADSVKG

SEQ ID NO: 143 - protein, 9 aa, Artificial Sequence
  Synthesized
TGWLGPFDY

SEQ ID NO: 144 - protein, 12 aa, Artificial Sequence
  Synthesized
RASQSVGSSYLA

SEQ ID NO: 145 - protein, 7 aa, Artificial Sequence
  Synthesized
GAFSRAT

SEQ ID NO: 146 - protein, 9 aa, Artificial Sequence
  Synthesized
QQYGSSPWT

SEQ ID NO: 147 - protein, 6 aa, Artificial Sequence
  Synthesized
NTYDMI

SEQ ID NO: 148 - protein, 16 aa, Artificial Sequence
  Synthesized
IITYSGSRYYANWAKG

SEQ ID NO: 149 - protein, 8 aa, Artificial Sequence
  Synthesized
DYMSGSHL

SEQ ID NO: 150 - protein, 11 aa, Artificial Sequence
  Synthesized
QASEDIYSFLA

SEQ ID NO: 151 - protein, 7 aa, Artificial Sequence
  Synthesized
SASSLAS

SEQ ID NO: 152 - protein, 12 aa, Artificial Sequence
  synthesized
QQGYGKNNVDNA

SEQ ID NO: 153 - protein, 6 aa, Artificial Sequence
  synthesized
NTYDMI

SEQ ID NO: 154 - protein, 16 aa, Artificial Sequence
  synthesized
IITYSGSRYYANWAKG

SEQ ID NO: 155 - protein, 8 aa, Artificial Sequence
  synthesized
DYMSGSHL

SEQ ID NO: 156 - protein, 11 aa, Artificial Sequence
  synthesized
QASEDIYSFLA

SEQ ID NO: 157 - protein, 7 aa, Artificial Sequence
  synthesized
SASSLAS

SEQ ID NO: 158 - protein, 12 aa, Artificial Sequence
  synthesized
QQGYGKNNVDNA

SEQ ID NO: 159 - protein, 6 aa, Artificial Sequence
  synthesized
SGYDMC

SEQ ID NO: 160 - protein, 18 aa, Artificial Sequence
  synthesized
CIAAGSAGITYDANWAKG

SEQ ID NO: 161 - protein, 11 aa, Artificial Sequence
  synthesized
SAFSFDYAMDL

SEQ ID NO: 162 - protein, 11 aa, Artificial Sequence
  synthesized
QASQSISSHLN

SEQ ID NO: 163 - protein, 7 aa, Artificial Sequence
  synthesized
KASTLAS

SEQ ID NO: 164 - protein, 12 aa, Artificial Sequence
  synthesized
QQGYSWGNVDNV

SEQ ID NO: 165 - protein, 6 aa, Artificial Sequence
  synthesized
STYDMI

SEQ ID NO: 166 - protein, 16 aa, Artificial Sequence
  synthesized
IISYVGNTYYASWAKG

SEQ ID NO: 167 - protein, 8 aa, Artificial Sequence
  synthesized
DFISGSHL

SEQ ID NO: 168 - protein, 11 aa, Artificial Sequence
  synthesized
QASESISSFLS

SEQ ID NO: 169 - protein, 7 aa, Artificial Sequence
  synthesized
SASTLAS

SEQ ID NO: 170 - protein, 12 aa, Artificial Sequence
  synthesized
QQGYSKSNVDNA

SEQ ID NO: 171 - protein, 6 aa, Artificial Sequence
  synthesized
SSYWMS

SEQ ID NO: 172 - protein, 16 aa, Artificial Sequence
  synthesized
VIDTNVYIYYANWAKG

SEQ ID NO: 173 - protein, 11 aa, Artificial Sequence
  synthesized
YVGNNDDYINL

SEQ ID NO: 174 - protein, 12 aa, Artificial Sequence
  Synthesized
QSSQSVYNGWLS

SEQ ID NO: 175 - protein, 7 aa, Artificial Sequence
  synthesized
GASTLAS

SEQ ID NO: 176 - protein, 11 aa, Artificial Sequence
  synthesized
LGSYTSSTENS

SEQ ID NO: 177 - protein, 6 aa, Artificial Sequence
  synthesized
SVINMG

SEQ ID NO: 178 - protein, 16 aa, Artificial Sequence
  synthesized
TITYVGNTYYASWAKG

SEQ ID NO: 179 - protein, 12 aa, Artificial Sequence
  synthesized
ESGTIYYSYFNL

SEQ ID NO: 180 - protein, 11 aa, Artificial Sequence
  synthesized
QASESISNYLS

SEQ ID NO: 181 - protein, 7 aa, Artificial Sequence
  synthesized
YASNLAS

SEQ ID NO: 182 - protein, 11 aa, Artificial Sequence
  synthesized
QSYYGGGSAYT

SEQ ID NO: 183 - protein, 6 aa, Artificial Sequence
  synthesized
SGYWIC

SEQ ID NO: 184 - protein, 18 aa, Artificial Sequence
  synthesized
CIYAGTSGSTSYASWARG

SEQ ID NO: 185 - protein, 8 aa, Artificial Sequence
  synthesized
NLYTYNSL

SEQ ID NO: 186 - protein, 13 aa, Artificial Sequence
  synthesized
QSSQSVYDNNWLA

SEQ ID NO: 187 - protein, 7 aa, Artificial Sequence
  synthesized
TVSTLAS

SEQ ID NO: 188 - protein, 12 aa, Artificial Sequence
  synthesized
QGTYYSSGWNFA

SEQ ID NO: 189 - protein, 6 aa, Artificial Sequence
  synthesized
SSYWMC

SEQ ID NO: 190 - protein, 17 aa, Artificial Sequence
  synthesized
CITTGSGSTYYASWAKR

SEQ ID NO: 191 - protein, 4 aa, Artificial Sequence
  synthesized
AFDL

SEQ ID NO: 192 - protein, 11 aa, Artificial Sequence
  synthesized
QASQSIYSYLN

SEQ ID NO: 193 - protein, 7 aa, Artificial Sequence
  synthesized
GASNLAS

SEQ ID NO: 194 - protein, 12 aa, Artificial Sequence
  synthesized
QSSWLSGAVGNA

SEQ ID NO: 195 - protein, 6 aa, Artificial Sequence
  synthesized
SSYGVS

SEQ ID NO: 196 - protein, 17 aa, Artificial Sequence
  synthesized
LIFPGIGFKDYASWVNG

SEQ ID NO: 197 - protein, 4 aa, Artificial Sequence
  Synthesized
DLDL

SEQ ID NO: 198 - protein, 12 aa, Artificial Sequence
  synthesized
QSSPSVYSNYLS

SEQ ID NO: 199 - protein, 7 aa, Artificial Sequence
  Synthesized
YASTLAS

SEQ ID NO: 200 - protein, 10 aa, Artificial Sequence
  Synthesized
AGGYSSSTRA

SEQ ID NO: 201 - protein, 6 aa, Artificial Sequence
  Synthesized
SSHWIC

SEQ ID NO: 202 - protein, 18 aa, Artificial Sequence
  Synthesized
CIYTGSIDVFYYASWAKG

SEQ ID NO: 203 - protein, 11 aa, Artificial Sequence
  Synthesized
AANTDTTYFNL

SEQ ID NO: 204 - protein, 11 aa, Artificial Sequence
  Synthesized
QASQSINNQLS

SEQ ID NO: 205 - protein, 7 aa, Artificial Sequence
  Synthesized
GASTLAS

SEQ ID NO: 206 - protein, 13 aa, Artificial Sequence
  Synthesized
HVHYCSGGSCFWA

(19) United States
(12) Patent Application Publication
(10) Pub. No.: US 2020/0347137 A1
(43) Pub. Date: Nov. 5, 2020

(54) BISPECIFIC ANTIBODIES AND METHODS OF MAKING AND USING THEREOF

(71) Applicants: SYSTIMMUNE, INC., Redmond, WA (US); SICHUAN BAILI PHARMACEUTICAL CO. LTD., Chengdu (CN)

(72) Inventors: Yi ZHU, Chengdu (CN); Katrina BYKOVA, Seattle, WA (US); Bill BRADY, Bothell, WA (US); Blair RENSHAW, Renton, WA (US); Dong XIA, Redmond, WA (US); Zeren GAO, Redmond, WA (US); Brian KOVACEVICH, Snohomish, WA (US); Jonathan K. FALLON, Quincy, MA (US); Phil TAN, Edmonds, WA (US)

(21) Appl. No.: 16/760,466

(22) PCT Filed: Nov. 2, 2018
(86) PCT No.: PCT/US2018/058810

§ 371 Date: Apr. 30, 2020

Related U.S. Application Data
(60) Provisional application No. 62/580,845, filed on Nov. 2, 2017.

(51) Int. Cl. C07K 16/28

(52) U.S. Cl. CPC C07K 16/2827; C07K 16/2818; C07K 2317/31; C07K 2317/10; C07K 2317/64; C07K 2317/92; C07K 2317/622

(57) ABSTRACT

The disclosure provides bispecific antibodies having the binding specificity to at least two of human CTLA4, PD-1 or PD-L1. In one embodiment, the bispecific antibody comprises IgG domains having heavy chains and light chains, and two scFv components being connected to either C-terminal of the heavy chains or N-terminal of the light chains, wherein the IgG domains have the binding specificity to a first antigen, wherein the scFv components have the binding specificity to a second antigen, and wherein the first antigen and the second antigen are different and are independently selected from a-CTLA4, α-PD-1, and α-PD-L1.

### CROSS REFERENCE TO RELATED APPLICATIONS

This application claims the benefit of U.S. Provisional Patent Application No. 62/580,845 filed on Nov. 2, 2017, titled “BISPECIFIC ANTIBODIES AND METHODS OF MAKING AND USING THEREOF”, which are expressly incorporated herein by reference in its entirety.

### TECHNICAL FIELD

The present disclosure generally relates to the technical field of biologic therapeutics, and more particularly relates to making and using bispecific antibodies. All references are incorporated herein by its entirety.

### BACKGROUND

Cancer cells develop various strategies to evade immunosurveillance. Absence of specific tumor antigens and loss of expression of major histocompatibility complex (MHC) molecules hinder the recognition of cancer cells by T lymphocytes. Immunosuppressive tumor microenvironment also contributes to the reduced recognition of tumor cells by the immune system. The tumor microenvironment is presented by immunosuppressive cellular populations composed of regulatory T cells, myeloid derived suppressor cells, tumor associated macrophages, suppressive B cells, immunosuppressive cytokines produced by tumor or stroma cells such as TGF-beta or IL-10, and immune checkpoint molecules that regulate T cell function [Marshall H T et al., Front Oncol 2018, 8:315].

Engaging a patient own immune system has been shown to be effective at controlling tumor growth and specific elimination of tumor cells while leaving normal tissue intact. Immunotherapy has provided an additional angle to treating cancer [Khalil D N et al., Adv Cancer Res 2015, 128:1-68].

Combining multiple modulators of the immune system is a new rapidly developing area of the immuno-oncology field. New therapeutic agents that can modulate immune response to tumor cells via multiple pathways can be greatly beneficial for cancer patient by increasing the patient response rate and in some cases decreasing toxicity.

Combination therapy with more than one monoclonal antibody targeting the immune system have been shown to be more efficacious in the treatment of cancer than treatments with single agents [Hellman M D et al., Adv Immunol 2016, 130: 251-77]. In addition to the increased efficacy and response rate, the combination therapy often has greater toxicity than a single agent treatment. Bispecific agents that modulate the immune system can be less toxic to patients and/or more potent, have additional mechanisms of action than treatments comprised of a combination of monoclonal antibodies with identical specificities.

### SUMMARY

The current disclosure relates to the bispecific antibodies, specifically, the bispecific antibodies that contain an IgG component therefore overcome fast clearance of BiTE molecule, having an advantage over CAR-T cell therapy as an off-the-shelf therapy that does not require ex vivo expansion of patients' immune cells. Another advantage of the bispecific antibodies is the enhanced ability to overcome suppressive tumor microenvironment by simultaneous engagement of two checkpoint receptors.

The bispecific antibodies in the current disclosure can be combined with other agents, for instance T-cell engagers, and further enhance their activity.

The present disclosure relates to bispecific antibodies that bind to two distinct targets expressed on immune and tumor cells. Both targets may be checkpoint antigens. In one embodiment, both targets may be checkpoint antigens on immune cells. In one embodiment, both targets may be checkpoint antigens on tumor cells. In one embodiment, one target is a checkpoint antigen on immune cells and another target is a checkpoint antigen on tumor cells. In one embodiment, the checkpoint antigen may be selected from PD-1, PD-L1 and CTLA4. In one embodiment, the targets may include any combination of PD-1, PD-L1 and CTLA4.

The disclosure further provides the composition of the bispecific agents and their therapeutic use for treatment of cancer and autoimmune deficiencies.

In one embodiment, the application discloses a bispecific antibody comprising IgG heavy chains and light chains, and two scFv components being connected to either C terminal of the heavy chains or N terminal of the light chains, wherein the IgG has the binding specificity to a first antigen, wherein the scFv components have the binding specificity to a second antigen, and wherein the first antigen and the second antigen are different and are independently selected from α-CTLA4, α-PD-1, and α-PD-L1.

In one embodiment, the bispecific antibody has the two scFv components connected to the C terminal of the heavy chain. In one embodiment, the first antigen comprises α-CTLA4 and the second antigen comprises α-PD-1 or α-PD-L1. In another embodiment, the first antigen comprises α-PD-1 or α-PD-L1 and the second antigen comprises α-CTLA4.

In one embodiment, the bispecific antibody has the two scFv components connected to the N terminal of the light chain. In one embodiment, the first antigen comprises α-PD-1 or α-PD-L1 and the second antigen comprises α-CTLA4. In another embodiment, the first antigen comprises α-CTLA4 and the second antigen comprises α-PD-1 or α-PD-L1.

In one embodiment, the bispecific antibody is an isolated monoclonal antibody.

In one embodiment, the bispecific antibody comprises an antigenic peptide sequence having a sequence as disclosed herein. In one embodiment, the bispecific antibody may have an antigenic peptide sequence having at least 70%, 80%, 90%, 95%, 98%, or 99% similarity with the disclosed amino acid sequences.

In one embodiment, the bispecific antibody comprises an antigen-binding fragment having a sequence as disclosed herein. In one embodiment, the bispecific antibody may have an antigen-binding fragment having a sequence with at least 70%, 80%, 90%, 98%, or 99% similarity with the disclosed antibody sequences.

In one embodiment, the bispecific antibody may have a binding affinity to α-CTLA4, α-PD-1 or α-PD-L1 with a Kd not greater than 70 nM, 50 nM, 40 nM, 30 nM, 20 nM, 10 nM, or 5 nM.

In one embodiment, the bispecific antibody may have a binding affinity to α-CTLA4 and α-PD-1 with a Kd not greater than 70 nM, 50 nM, 40 nM, 30 nM, 20 nM, 10 nM, or 5 nM.

In one embodiment, the bispecific antibody may have a binding affinity to α-CTLA4 and α-PD-L1 with a Kd not greater than 70 nM, 50 nM, 40 nM, 30 nM, 20 nM, 10 nM, or 5 nM.

In one embodiment, the bispecific antibody may have a binding affinity to two of α-CTLA4, α-PD-1, or α-PD-L1 with a Kd not greater than 70 nM, 50 nM, 40 nM, 30 nM, 20 nM, 10 nM, or 5 nM.

In one embodiment, the bispecific antibody may exhibit one or more functional properties selected from high affinity binding to α-CTLA4, α-PD-1, or α-PD-L1, inhibiting binding of PD-L1 to PD-1, enhancing T cell activation, the ability to stimulate antibody responses and/or the ability to reverse the suppressive function of immunosuppressive cells, such as T regulatory cells.

In one embodiment, enhancing T-cell activation comprises T-cell proliferation, IFN-γ and/or IL-2 secretion, or a combination thereof.

In one embodiment, the bispecific antibody comprising a human framework region.

In one embodiment, the bispecific antibody may be a humanized antibody, a chimeric antibody, or a recombinant antibody.

In one embodiment, the bispecific antibody comprises an IgG1 constant region to extend the circulating half-life of the bispecific molecules. In one embodiment, the IgG1 constant region of the bispecific antibody comprises an amino acid sequence having at least 98% similarity with SEQ ID No. 136.

In one embodiment, the application discloses an isolated bispecific antibody selected from the group consisting of those clones described or having the described characteristics as disclosed herein.

In one embodiment, the application discloses an IgG1 heavy chains for the bispecific antibody, comprising an amino acid sequence selected from sequences as disclosed herein. In one embodiment, the IgG1 heavy chains may have an amino acid sequence having at least 70%, 80%, 90%, 98%, or 99% similarity with SEQ ID NO. 02, 06, 08, 10, 12, 14, 16, 18, 20, 22, 26, 30, 34, 38, 42, 46, 50, 54, 58, 62, 72, 92, 96, 100, 104, 108, 112, 116, 120, 124, 128, or 132.

In one embodiment, the application discloses a kappa light chain for the bispecific antibody. In one embodiment, the kappa light chain may have an amino acid sequence having at least 70%, 80%, 90%, 98%, or 99% similarity with SEQ ID NO. 04, 28, 32, 36, 40, 44, 48, 52, 56, 60, and 64.

In one embodiment, the application discloses a variable light chain for the bispecific antibody, comprising an amino acid sequence as disclosed herein. In one embodiment, the variable light chain may have an amino acid sequence having at least 70%, 80%, 90%, 98%, or 99% similarity with SEQ ID NO. 94, 98, 102, 106, 110, 114, 118, 122, 126, 130, or 134.

In one embodiment, the application discloses a variable heavy chain for the bispecific antibody, comprising an amino acid sequence as disclosed herein. In one embodiment, the variable heavy chain may have an amino acid sequence having at least 70%, 80%, 90%, 98%, or 99% similarity with SEQ ID NO. 92, 96, 100, 104, 108, 112, 116, 120, 124, 128, or 132.

In one embodiment, the application discloses an isolated nucleic acid encoding the bispecific antibody, comprising the IgG1 heavy chain disclosed herein, the kappa light chain disclosed herein, the variable light chain disclosed herein, or the variable heavy chain disclosed herein. In one embodiment, the application discloses an isolated nucleic acid encoding the bispecific antibody, comprising the IgG1 heavy chain having an amino acid sequence having at least 70%, 80%, 90%, 98%, or 99% similarity with SEQ ID NO. 02, 06, 08, 10, 12, 14, 16, 18, 20, 22, 26, 30, 34, 38, 42, 46, 50, 54, 58, 62, 72, 92, 96, 100, 104, 108, 112, 116, 120, 124, 128, or 132, the kappa light chain having an amino acid sequence having at least 70%, 80%, 90%, 98%, or 99% similarity with SEQ ID NO. 04, 28, 32, 36, 40, 44, 48, 52, 56, 60, and 64, the variable light chain having an amino acid sequence having at least 70%, 80%, 90%, 98%, or 99% similarity with SEQ ID NO. 94, 98, 102, 106, 110, 114, 118, 122, 126, 130, or 134, or the variable heavy chain having an amino acid sequence having at least 70%, 80%, 90%, 98%, or 99% similarity with SEQ ID NO. 92, 96, 100, 104, 108, 112, 116, 120, 124, 128, or 132.

In one embodiment, the application discloses an expression vector comprising the isolated nucleic acid disclosed herein. In one embodiment, the expression vector comprises an isolated nucleic acid having a sequence having at least 70%, 80%, 90%, 98%, or 99% similarity with the nucleic acid sequence disclosed herein.

In one embodiment, the expression vector is expressible in a cell.

In one embodiment, the application discloses a host cell comprising the nucleic acid of disclosed herein.

In one embodiment, the application discloses a host cell comprising the expression vector.

In some embodiments, the application discloses the host cell, wherein the host cell is a prokaryotic cell or a eukaryotic cell.

In some embodiments, the application discloses a method of producing an antibody comprising culturing the host cell provided thereof so that the antibody is produced.

In some embodiments, the application discloses an immunoconjugate comprising the bispecific antibody and a cytotoxic agent. In some embodiments, the cytotoxic agent is a chemotherapeutic agent, a growth inhibitory agent, a toxin, or a radioactive isotope.

In one embodiment, the application discloses a pharmaceutical composition, comprising the bispecific antibody and a pharmaceutically acceptable carrier. In one embodiment, the application discloses a pharmaceutical composition, comprising the immunoconjugate and a pharmaceutically acceptable carrier. In one embodiment, the pharmaceutical composition further comprises radioisotope, radionuclide, a toxin, a therapeutic agent, a chemotherapeutic agent or a combination thereof.

In one embodiment, the application discloses a method of treating a subject with a cancer, comprising administering to the subject an effective amount of the bispecific antibody disclosed herein. In one embodiment, the cancer comprises breast cancer, colorectal cancer, pancreatic cancer, head and neck cancer, melanoma, ovarian cancer, prostate cancer, non-small lung cell cancer, small cell lung cancer, glioma, esophageal cancer, nasopharyngeal cancer, kidney cancer, gastric cancer, liver cancer, bladder cancer, cervical cancer, brain cancer, lymphoma, leukaemia, myeloma.

In one embodiment, the application discloses the method of treating a subject with a cancer, wherein the cancer comprises cells expressing PD-L1.

In one embodiment, the application discloses the method of treating a subject with a cancer, further comprising co-administering an effective amount of a therapeutic agent. In some embodiments, the therapeutic agent comprises an antibody, a chemotherapy agent, an enzyme, or a combination thereof.

In some embodiments, the therapeutic agent comprises capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

In one embodiment, the application discloses the method of treating a subject with a cancer, wherein the subject is a human.

In one embodiment, the application discloses a solution comprising an effective concentration of the bispecific antibody disclosed herein, wherein the solution is blood plasma in a subject.

Still other embodiments of the present application will become readily apparent to those skilled in the art from the following detailed description, wherein is described embodiments of the application by way of illustrating the best mode contemplated for carrying out the application. As will be realized, the application is capable of other and different embodiments and its several details are capable of modifications in various obvious respects, all without departing from the spirit and the scope of the present application. Accordingly, the drawings and detailed description are to be regarded as illustrative in nature and not as restrictive.

### BRIEF DESCRIPTION OF THE DRAWINGS

The foregoing and other features of this disclosure may become more fully apparent from the following description and appended claims, taken in conjunction with the accompanying drawings. Understanding that these drawings depict only several embodiments arranged in accordance with the disclosure and are, therefore, not to be considered limiting of its scope, the disclosure may be described with additional specificity and detail through use of the accompanying drawings, in which:

FIG. 1 shows a diagram of example bispecific antibodies targeting CTLA4, PD-1 and PD-L1 antigens;

FIG. 2 shows binding of example anti-PD-1 antibodies to PD-1 antigen expressed on the surface of CHO cell line;

FIG. 3 depicts results from a biochemical assay assessing the ability of the representative bispecific antibodies to block the interaction between CTLA4 and CD80;

FIG. 4 shows stimulation of PBMC with super antigen SEB; FIG. 4A shows the treatment with PD224D1×CT4 IgG1 null and PL23006×CT4 IgG1 null; FIG. 4B shows the treatment with CT4×PD224D1 IgG1 null, CT4×PL221G5 IgG1 and CT4×PL221G5 IgG1 null;

FIG. 5 shows results from a signaling assay for PD-1/PDL-1 pathway;

FIG. 6 shows results from Dendritic Cell Mixed Lymphocytes Reaction study;

FIG. 7 shows results from regulatory T cell suppression assay; FIG. 7A shows the proliferation of CD8+ T cells; and FIG. 7B shows IFNg production;

FIG. 8 shows CD8 T cell degranulation in response to treatment with example bispecific antibodies;

FIG. 9 shows results from MiXeno HCC287 mouse tumor model;

FIG. 10 shows effect of example bispecific antibodies on proliferation of Flu-specific CD8 T cells;

FIG. 11 shows results from PBMC memory response to CEFT peptide pool; and

FIG. 12 shows enhancement of Redirected T Cell Cytotoxicity by example bispecific antibodies.

### DETAILED DESCRIPTION

In the following detailed description of embodiments of the application, reference is made to the accompanying drawings in which like references indicates similar elements, and in which is shown by way of illustration, specific embodiments in which the application may be practiced. These embodiments are described in sufficient detail to enable those skilled in the art to practice the application. In other instances, well-known circuits, structures, and techniques have not been shown in detail in order not to obscure the understanding of this description. The following detailed description is, therefore, not to be taken in a limiting sense, and the scoop of the application is defined only by the appended claims.

The disclosure relates to bispecific antibodies that specifically bind to human CTLA4, PD-1 or PD-L1. In some embodiments, the bispecific antibody comprises of a first arm that binds to CTLA4 and a second arm that binds to PD-1 or PD-L1. In some embodiments, the bispecific antibody comprises of a first arm that binds to PD-1 or PD-L1 and a second arm that binds to CTLA4. Examples of domains that comprise the arms include, but are not limited to, Fab and scFv domains. Each arm contains two antigen-binding domains and is connected to another arm via Fc domain. The Fc domain can be of human IgG1, IgG2, IgG3, IgG4 or an engineered isotype.

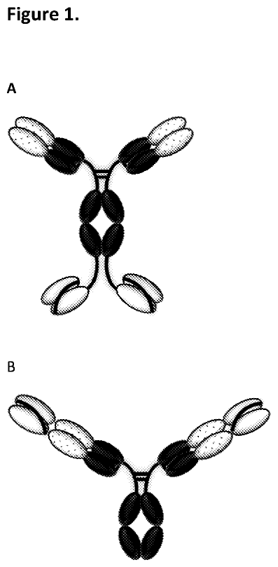

The bispecific antibodies of this application (FIG. 1) target human CTLA4, human PD-1 and human PD-L1. Each of these targeted bispecific antibodies carry an anti-CTLA4 (SEQ IDs 91, 93) and an anti-human PD-1 (SEQ IDs 95, 97, 131, 133) or PD-L1 binding domains (SEQ IDs 99, 101, 103, 105, 107, 109, 111, 113, 115, 117, 119, 121, 123, 125, 127, 129). Either one of the binding domains was converted to scFv (VH-VL orientation) for placement at the C-terminus, or scFv (VL-VH orientation) for placement at the N-terminus (FIGS. 1A and B respectively).

In some embodiments, scFv molecules described herein contain a 20-amino acid flexible gly-gly-gly-gly-ser (G4S) X4 linker that operably links the VH and VL, regardless of the V-region orientation (LH or HL). The remaining positions in the bispecific antibody may be consist of a human IgG1 Fc or IgG1 null Fc heavy chain, VH-CH1-Hinge-CH2-CH3, and its corresponding kappa light chain, VL-CL. scFv domains were genetically linked through a 10-amino acid (G4S)×2 linker to either N-terminal or C-terminal of IgG1 heavy chain, resulting in a contiguous ˜100 kDa heavy chain monomer peptide. When co-transfected with the appropriate light chain, the final symmetric bispecific molecule may be purified through the human IgG1 Fc (Protein A) and assayed to assess functional activity.

Heavy and light chain gene “cassettes” were previously constructed such that V-regions could be cloned using either restriction enzyme sites (FIG. 1A example: HindIII/NotI for the heavy chain and HindIII/BsiWI for the light chain). In one embodiment, “restriction-free cloning” NEBuilder (NEB, Ipswich, Mass.) was used.

Bispecific antibodies are produced through a process that involves design of the intact molecule, synthesis and cloning of the nucleotide sequences for each domain, expression in mammalian cells and purification of the final product. Nucleotide sequences were assembled using the Geneious 10.2.3 software package (Biomatters, Auckland, NZ) and broken up into their component domains for gene synthesis (Genewiz, South Plainsfield, N.J.). In one example (FIG. 1A), the heavy chain of CT4×PD224D1 IgG1 null bispecific antibody (SEQ ID 2) consists of the anti-CTLA4 VH domain fused to the human IgG1 null Fc domain (hinge, CH1, and CH2), followed by a 10-amino acid (G4S)×2 linker fused to the anti-PD-1 scFV domain (clone PD224D1). Using NEBuilder web-based tools, 5′ and 3′ nucleotides were appended to each of the domains so that each domain overlaps its flanking domains by 20-30 nucleotides, which direct site-specific recombination, thus genetically fusing each domain in a single gene assembly step.

The light chain of CT4×PD224D1 IgG1 null consists of the anti-CTLA4 VL domain fused to the human C kappa domain. A synthesized gene fragment was digested with the restriction enzymes HindIII and BsiWI and was then ligated in-frame with the human C kappa domain. For both constructs, a small aliquot was transformed into E. coli DH10b (Invitrogen, Carlsbad, Calif.) and plated on TB+carbenicillin 100 ug/ml plates (Teknova, Hollister, Calif.) and incubated at 37 C overnight. Resultant colonies were selected and 2 ml overnight cultures inoculated in TB+carbenicillin. DNA was prepared (Thermo-Fisher, Carlsbad, Calif.) from overnight cultures and subsequently sequenced (Genewiz, South Plainsfield, N.J.) using sequencing primers (Sigma, St. Louis, Mo.) flanking each domain. All DNA sequences were assembled and analysed in Geneious.

### EXAMPLES

### Example 1: Binding of Anti-PD-L1 Antibodies to PD-L1 Antigen

Binding affinities and kinetics of anti-PD-L1 antibodies to PD-L1 recombinant protein were assessed via Surface Plasmon Resonance on ForteBio Octet RED96 instrument. The antigen was immobilized on the sensor chip surface and the tested antibodies were flown over the immobilized antigens. All molecules showed strong binding to the antigen as shown in Table 1 for examples.

TABLE 1
Binding of Anti-PD-L1 Antibodies to PD-L1 Antigen

| | mAb | | | scFv-Fc | | | Fc-scFv | | |
|---|---|---|---|---|---|---|---|---|---|
| Sample ID | KD (M) | kon (1/Ms) | kdis (1/s) | KD (M) | kon (1/Ms) | kdis (1/s) | KD (M) | kon (1/Ms) | kdis (1/s) |
| PL004B9 | 1.37E−09 | 5.08E+05 | 6.95E−04 | 1.42E−09 | 3.87E+05 | 5.50E−04 | 1.54E−09 | 3.07E+05 | 4.71E−04 |
| PL221G5 | 4.83E−10 | 5.72E+05 | 2.76E−04 | 5.97E−10 | 5.08E+05 | 3.03E−04 | 4.12E−10 | 4.14E+05 | 1.71E−04 |
| PL230C6 | 7.69E−10 | 6.09E+05 | 4.68E−04 | 9.17E−10 | 5.03E+05 | 4.61E−04 | 8.68E−10 | 4.18E+05 | 3.62E−04 |
| PL231H2 | 7.81E−10 | 5.01E+05 | 3.91E−04 | 9.99E−10 | 4.20E+05 | 4.19E−04 | 4.62E−10 | 8.40E+05 | 3.89E−04 |

### Example 2: Binding of Anti-PD-1 Antibodies to PD-1 Antigen

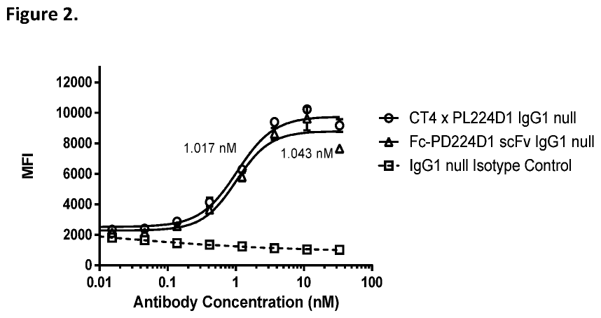

Binding of the bispecific antibodies and their components to PD-1 antigen expressed on the surface of CHO cell line was assessed using FACS method. The bispecific antibodies were incubated with CHO cell line expressing PD-1 antigen and then detected with secondary anti-human antibodies directly conjugated to Alexa Fluor 647 fluorochrome. Cellular binding of the test antibodies was analyzed on a flow cytometer BD LSRFortessa. All tested antibodies bound to the antigen with a KD in a single digit nanomolar range (FIG. 2).

### Example 3: Blockade of Interaction Between CTLA4 and CD80

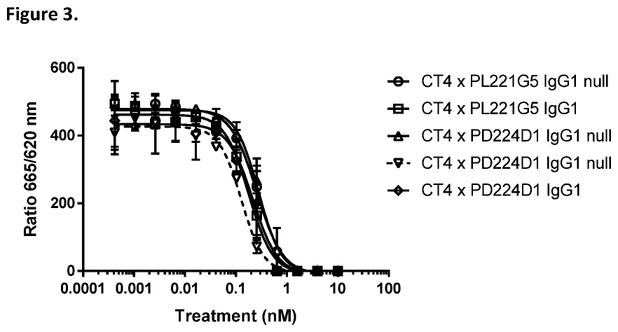

Ability of the bispecific antibodies to block the interaction between CTLA4 and its ligand CD80 was tested in a biochemical interaction assay (Cisbio). Briefly, a bispecific antibody was incubated with CTLA4 and CD80 proteins. Detection antibodies recognizing CTLA4 and CD80 proteins and labeled with HTRF donor/acceptor fluorescent pair then were added to the mixture. The interaction between CTLA4 and CD80 was assessed via FRET efficiency. All bispecific antibodies tested were able to block the interaction between CTLA4 and CD80 (FIG. 3).

### Example 4: Stimulation of PBMC with Superantigen SEB

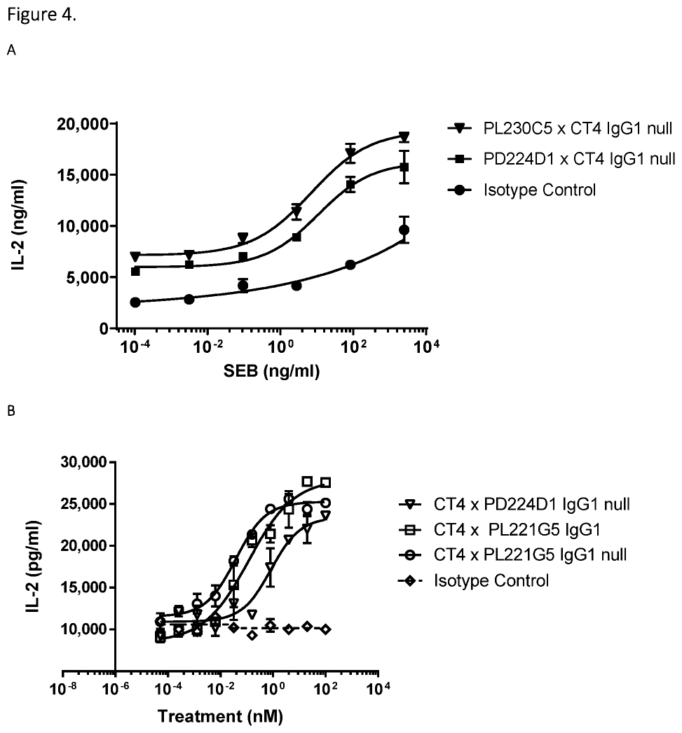

Ability of the bispecific antibodies to enhance cytokine release from human Peripheral Blood Mononuclear Cells (PBMC) after stimulation with superantigen SEB was assessed. All bispecific molecules were able to significantly enhance the production of IL-2 by PBMC upon stimulation with SEB, as shown in FIG. 4A and FIG. 4B.

### Example 5: PD-1/PD-L1 Pathway Signaling

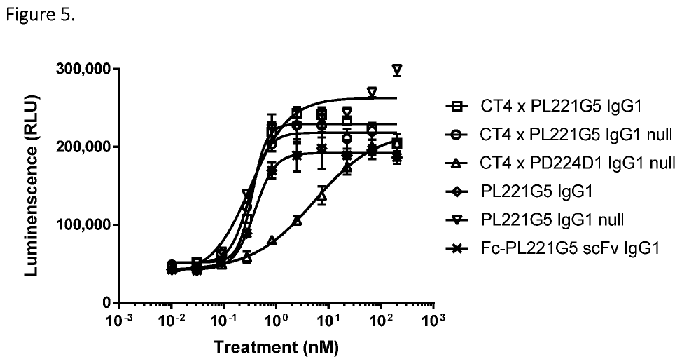

The bispecific antibodies were tested for their ability to block PD-1/PD-L1 pathway. Briefly, the test molecules were incubated with Jurkat reporter cell line expressing PD-1 receptor and luciferase NFAT reporter and CHO-PD-L1 cell line (Promega). The ability of the test antibodies to block the signaling through the PD-1/PD-L1 pathway was assessed via an increase in NFAT signaling. The NFAT signaling in turn was monitored via activity of luciferase reporter gene. The assay was read on a plate reader (Clariostar, BMG). All tested bispecific antibodies and monoclonal antibody controls were able to block PD-1/PD-L1 signaling (FIG. 5).

### Example 6: Dendritic Cell Mixed Leukocyte Reaction

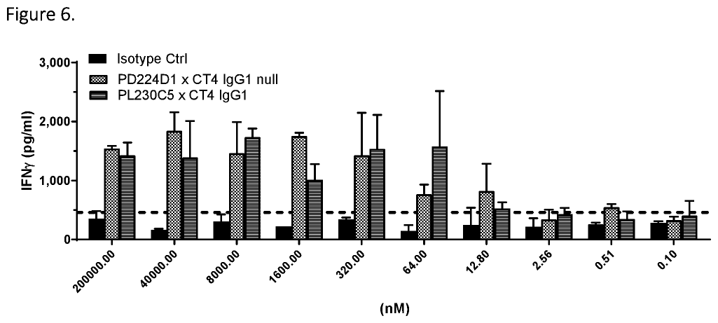

The bispecific antibodies were tested for their ability to enhance Dendritic cell Mixed Leucocyte Reaction (MLR). The test molecules were incubated for 6 days with dendritic cells from one donor and T cells isolated from another donor. Dendritic cells were differentiated in vitro from monocytes in the presence of GM-CSF and IL-4. Monocytes and T cell populations were isolated from PBMC with StemCell isolation kits. The ability of the test molecules to enhance MLR was assessed via secreted IFNγ. All tested bispecific antibodies were able to augment production of IFNγ as shown on FIG. 6.

### Example 7: Suppression of CD8 T Cells by Regulatory T Cells

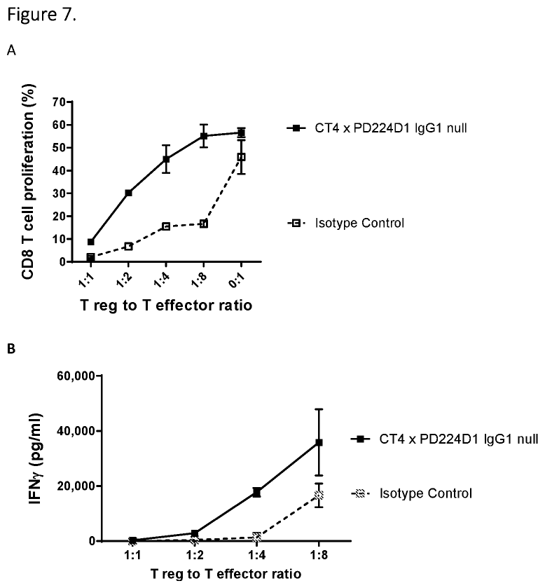

The bispecific antibodies were tested for their ability to block suppressive effect of regulatory T cells on effector CD8 T cell proliferation and cytokine production. CD8 T cells were isolated with StemCell isolation kit and labeled with CellTrace dye (ThermoFisher). Dendritic cells were prepared as described earlier in the MLR study. Regulatory T cells were isolated from PBMC with StemCell isolation kit and expanded in vitro. The bispecific antibody was incubated with effector CD8 T cells, dendritic cells and regulatory T cells for 4 days. The ability of the bispecific antibody to rescue effector CD8 T cell function in the presence of regulatory T cells was assessed via proliferation of effector CD8 T cells (FIG. 7A) and secreted IFNγ (FIG. 7B).

### Example 8: CD8 T Cell Degranulation

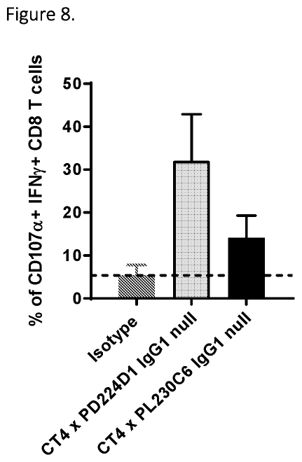

Ability of bispecific molecules to have an effect on cytotoxic CD8 T cells was assessed in this study. Briefly, CD8 T cells were purified with StemCell isolation kit and stimulated with CEFT peptide pool (JPT Peptide Technologies) in the presence of the bispecific test molecules. The media was supplemented with IL-7 and IL-21. On day 11 CD8 T cells were re-stimulated with the peptides in the presence of Brefeldin and Monensin and anti-CD107a antibody directly labeled with a fluorochrome. 24 hours later CD8 T cells were stained with anti-IFNγ antibodies directly conjugated to a fluorochrome and assessed on a flow cytometer BD LSRFortessa. As shown on FIG. 8, all tested bispecific antibodies were able to increase the number of cytotoxic IFNγ positive T cells.

### Example 9: MiXeno Mouse Tumor Model

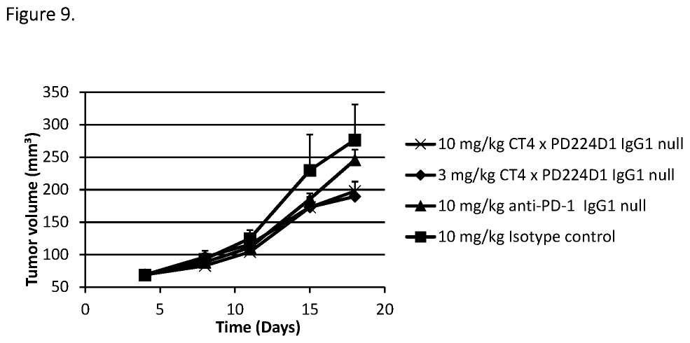

Humanized mouse model was used to assess the ability of bispecific antibodies of this class to inhibit tumor growth in vivo. Briefly, NOG mice were reconstituted with human PBMC (5×10⁶ cells per mouse). On day 3 the animals were subcutaneously inoculated with a human lung cancer cell line HCC827 (0.5E6 cells/animal) and started on a biweekly treatment with a bispecific antibody and control antibodies. Tumor volumes were measured every 2-3 days. Animal weight was monitored. The tested bispecific antibody was able to inhibit tumor growth better than the control antibodies (FIG. 9).

### Example 10: Proliferation of Flu-Specific T Cells

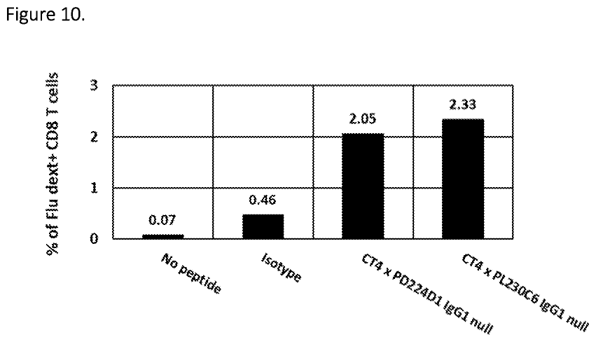

Ability of the bispecific antibodies to augment proliferation of antigen specific CD8 T cells was assessed in this study. CD8 T cells were purified from PBMC with StemCell isolation kit, pulsed with influenza specific peptides (JPT Peptide Technologies) and incubated in the presence of the bispecific antibodies for 14 days. The media was supplemented with IL-7 and IL-21. On day 15 the cells were stained with a peptide specific MHC dextramers (Immudex) and assessed on a flow cytometer BD LSRFortessa. All tested bispecific antibodies were able to increase the number of antigen specific CD8 T cells (FIG. 10).

### Example 11: PBMC Memory Response to CEFT Peptide Pool

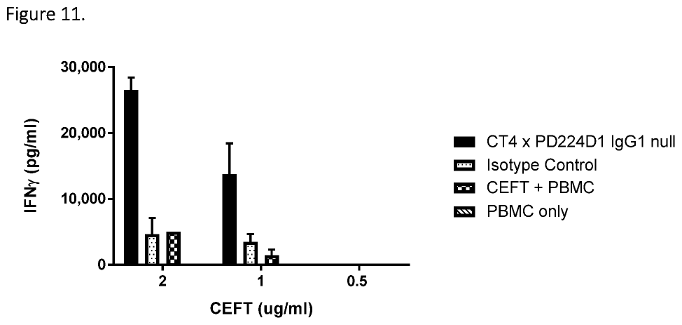

Ability of the bispecific antibodies to augment T cell memory response was assessed. Briefly, PBMC were incubated for 4-5 days in the presence of peptides specific for CMV, EBV, Influenza and Tetanus (JPT Peptide Technologies). The amount of secreted IFNγ was quantified. The bispecific CT4×PD224D1, shown on FIG. 11, was able to enhance production of IFNγ several fold over the control treatment.

### Example 12: Enhancement of Redirected T Cell Cytotoxicity

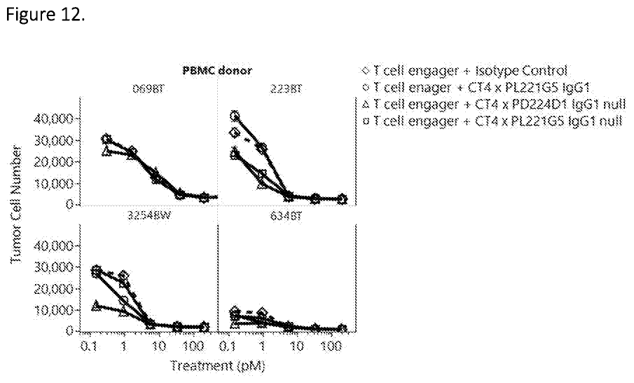

The bispecific antibodies were tested for their ability to enhance Redirected T cell Cytotoxicity (RTCC) against a tumor cell line target. The tumor cell line was stably expressing nucleus-localized Red Fluorescent Protein (RFP) delivered via lentiviral transduction (Sartorius). The tumor cells were co-cultured with PBMC and a T cell engager molecule specific for the given tumor cell line. The bispecific antibodies were added to the co-cultures. Lysis of tumor cells was assessed by counting RFP labeled tumor cell nuclei. Images were acquired on live cell imager IncuCyte (Sartorius). Activity of the antibodies were assessed after 96 hours of incubation. Four PBMC donors were tested in this study. All bispecific antibodies tested were able to enhance RTCC activity in at least one PBMC donor tested (FIG. 12).

While the present disclosure has been described with reference to particular embodiments or examples, it may be understood that the embodiments are illustrative and that the disclosure scope is not so limited. Alternative embodiments of the present disclosure may become apparent to those having ordinary skill in the art to which the present disclosure pertains. Such alternate embodiments are considered to be encompassed within the scope of the present disclosure. Accordingly, the scope of the present disclosure is defined by the appended claims and is supported by the foregoing description. All references cited or referred to in this disclosure are hereby incorporated by reference in their entireties.

SEQUENCE LISTING

| SEQ ID | Description |
|---|---|
| 1 | CT4 x PD224D1 nucleotide |
| 2 | CT4 x PD224D1 amino acid |
| 3 | CT4 Light Chain nucleotide |
| 4 | CT4 Light Chain amino acid |
| 5 | CT4 x PL23006 nucleotide |
| 6 | CT4 x PL23006 amino acid |
| 7 | CT4 x PL221G5 nucleotide |
| 8 | CT4 x PL221G5 amino acid |
| 9 | CT4 x PL231H2 nucleotide |
| 10 | CT4 x PL231H2 amino acid |
| 11 | CT4 x PL004B5 nucleotide |
| 12 | CT4 x PL004B5 amino acid |
| 13 | CT4 x PL004B9 nucleotide |
| 14 | CT4 x PL004B9 amino acid |
| 15 | CT4 x PD206F12 nucleotide |
| 16 | CT4 x PD206F12 amino acid |
| 17 | CT4 x PD215A1 nucleotide |
| 18 | CT4 x PD215A1 amino acid |
| 19 | CT4 x PD220F6 nucleotide |
| 20 | CT4 x PD220F6 amino acid |
| 21 | CT4 x PD225G11 nucleotide |
| 22 | CT4 x PD225G11 amino acid |
| 23 | Gly 4 Ser x 4 nucleotide |
| 24 | Gly 4 Ser x 4 amino acid |
| 25 | PL23006 x CT4 nucleotide |
| 26 | PL23006 x CT4 amino acid |
| 27 | PL23006 Light Chain nucleotide |
| 28 | PL23006 Light Chain amino acid |
| 29 | PD224D1 x CT4 nucleotide |
| 30 | PD224D1 x CT4 amino acid |
| 31 | PD224D1 Light Chain nucleotide |
| 32 | PD224D1 Light Chain amino acid |
| 33 | PL221G5 x CT4 |
| 34 | PL221G5 x CT4 |
| 35 | PL221G5 Light Chain |
| 36 | PL221G5 Light Chain |
| 37 | PL231H2 x CT4 nucleotide |
| 38 | PL231H2 x CT4 amino acid |
| 39 | PL231H2 Light Chain nucleotide |
| 40 | PL231H2 Light Chain amino acid |
| 41 | PL004B5 x CT4 nucleotide |
| 42 | PL004B5 x CT4 amino acid |
| 43 | PL004B5 Light Chain nucleotide |
| 44 | PL004B5 Light Chain amino acid |
| 45 | PL004B9 x CT4 nucleotide |
| 46 | PL004B9 x CT4 amino acid |
| 47 | PL004B9 Light Chain nucleotide |
| 48 | PL004B9 Light Chain amino acid |
| 49 | PD206F12 x CT4 nucleotide |
| 50 | PD206F12 x CT4 amino acid |
| 51 | PD206F12 Light Chain nucleotide |
| 52 | PD206F12 Light Chain amino acid |
| 53 | PD215A1 x CT4 nucleotide |
| 54 | PD215A1 x CT4 amino acid |
| 55 | PD215A1 Light Chain nucleotide |
| 56 | PD215A1 Light Chain amino acid |
| 57 | PD220F6 x CT4 nucleotide |
| 58 | PD220F6 x CT4 amino acid |
| 59 | PD220F6 Light Chain nucleotide |
| 60 | PD220F6 Light Chain amino acid |
| 61 | PD225G11 x CT4 nucleotide |
| 62 | PD225G11 x CT4 amino acid |
| 63 | PD225G11 Light Chain nucleotide |
| 64 | PD225G11 Light Chain amino acid |
| 65 | CT4 signal peptide nucleotide |
| 66 | CT4 signal peptide amino acid |
| 67 | Heavy Chain signal peptide nucleotide |
| 68 | Heavy Chain signal peptide amino acid |
| 69 | Light Chain signal peptide nucleotide |
| 70 | Light Chain signal peptide amino acid |
| 71 | CT4 scFv nucleotide |
| 72 | CT4 scFv amino acid |
| 73 | PD224D1 scFv nucleotide |
| 74 | PD224D1 scFv amino acid |
| 75 | PD206F12 scFv nucleotide |
| 76 | PD206F12 scFv amino acid |
| 77 | PD215A1 scFv nucleotide |
| 78 | PD215A1 scFv amino acid |
| 79 | PD225G11 scFv nucleotide |
| 80 | PD225G11 scFv amino acid |
| 81 | PL23006 scFv nucleotide |
| 82 | PL23006 scFv amino acid |
| 83 | PL231H2 scFv nucleotide |
| 84 | PL231H2 scFv amino acid |
| 85 | PL221G5 scFv nucleotide |
| 86 | PL221G5 scFv amino acid |
| 87 | PL004B5 scFv nucleotide |
| 88 | PL004B5 scFv amino acid |
| 89 | PL004B9 scFv nucleotide |
| 90 | PL04B9 scFv amino acid |
| 91 | CT4 VH nucleotide |
| 92 | CT4 VH amino acid |
| 93 | CT4 VL nucleotide |
| 94 | CT4 VL amino acid |
| 95 | PD224D1 VH nucleotide |
| 96 | PD224D1 VH amino acid |
| 97 | PD224D1 VL nucleotide |
| 98 | PD224D1 VL amino acid |
| 99 | PD206F12 VH nucleotide |
| 100 | PD206F12 VH amino acid |
| 101 | PD206F12 VL nucleotide |
| 102 | PD206F12 VL amino acid |
| 103 | PD215A1 VH nucleotide |
| 104 | PD215A1 VH amino acid |
| 105 | PD215A1 VL nucleotide |
| 106 | PD215A1 VL amino acid |
| 107 | PD225G11 VH nucleotide |
| 108 | PD225G11 VH amino acid |
| 109 | PD225G11 VL nucleotide |
| 110 | PD225G11 VL amino acid |
| 111 | PL23006 VH nucleotide |
| 112 | PL23006 VH amino acid |
| 113 | PL23006 VL nucleotide |
| 114 | PL23006 VL amino acid |
| 115 | PL231H2 VH nucleotide |
| 116 | PL231H2 VH amino acid |
| 117 | PL231H2 VL nucleotide |
| 118 | PL231H2 VL amino acid |
| 119 | PL221G5 VH nucleotide |
| 120 | PL221G5 VH amino acid |
| 121 | PL221G5 VL nucleotide |
| 122 | PL221G5 VL amino acid |
| 123 | PL004B5 VH nucleotide |
| 124 | PL004B5 VH amino acid |
| 125 | PL004B5 VL nucleotide |
| 126 | PL004B5 VL amino acid |
| 127 | PL004B9 VH nucleotide |
| 128 | PL004B9 VH amino acid |
| 129 | PL004B9 VL nucleotide |
| 130 | PL004B9 VL amino acid |
| 131 | PD220F6 VH nucleotide |
| 132 | PD220F6 VH amino acid |
| 133 | PD220F6 VL nucleotide |
| 134 | PD220F6 VL amino acid |
| 135 | Human IgG1 Constant Domain nucleotide |
| 136 | Human IgG1 Constant Domain amino acid |
| 137 | Human IgG1 Constant Domain null nucleotide |
| 138 | Human IgG1 Constant Domain null amino acid |
| 139 | PD220F6 scFv nucleotide |
| 140 | PD220F6 scFv amino acid |

SEQ ID 01: CT4 x PD224D1 nucleotide sequence (nt)
CAGGTGCAGCTGGTGGAGTCTGGGGGAGGCGTGGTCCAGCCTGGGAGGTCCCTGAGACTCTCCTGTGCAGCCTCTGGATTCA
CCTTCAGTAGCTATACTATGCACTGGGTCCGCCAGGCTCCAGGCAAGGGGCTGGAGTGGGTGACATTTATATCATATGAT
GGAAACAATAAATACTACGCAGACTCCGTGAAGGGCCGATTCACCATCTCCAGAGACAATTCCAAGAACACGCTGTATCT
GCAAATGAACAGCCTGAGAGCTGAGGACACGGCTATATATTACTGTGCGAGGACCGGCTGGCTGGGGCCCTTTGACTACT
GGGGCCAGGGAACCCTGGTCACCGTCTCCTCAGCTAGCACCAAGGGCCCATCGGTCTTCCCCCTGGCACCCTCCTCCAAG
AGCACCTCTGGGGGCACAGCGGCCCTGGGCTGCCTGGTCAAGGACTACTTCCCCGAACCGGTGACGGTGTCGTGGAACTC
AGGCGCCCTGACCAGCGGCGTGCACACCTTCCCGGCTGTCCTACAGTCCTCAGGACTCTACTCCCTCAGCAGCGTGGTGA
CCGTGCCCTCCAGCAGCTTGGGCACCCAGACCTACATCTGCAACGTGAATCACAAGCCCAGCAACACCAAGGTGGACAAG
AGAGTTGAGCCCAAATCTTGTGACAAAACTCACACATGCCCACCGTGCCCAGCACCTGAAGCCGCGGGGGCACCGTCAGT
CTTCCTCTTCCCCCCAAAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGGAGTACAA
GTGCGCGGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGATGAGCTGACCAAGAACCAGGTCAGCCTGACCTGCCTGGTCAAAGGCTTCTAT
CCCAGCGACATCGCCGTGGAGTGGGAGAGCAATGGGCAGCCGGAGAACAACTACAAGACCACGCCTCCCGTGCTGGACTC
CGACGGCTCCTTCTTCCTCTATAGCAAGCTCACCGTGGACAAGAGCAGGTGGCAGCAGGGGAACGTCTTCTCATGCTCCG
TGATGCATGAGGCTCTGCACAACCACTACACGCAGAAGAGCCTCTCCCTGTCTCCGGGTGGCGGTGGAGGGTCCGGCGGT
GGTGGATCCGAGGTGCAGCTGGTGGAGTCTGGGGGAGGCTTGGTCCAGCCTGGGGGGTCCCTGAGACTCTCCTGTACAGC
CTCTGGATTCTCCCTCAGTAGCTATGCAATGAGCTGGGTCCGCCAGGCTCCAGGGAAGGGGCTGGAGTACATCGGCTACA
TTGGTGATACTACTGGCATAGCCTACGCGAGCTGGGCGAATGGCAGATTCACCATCTCCAAAGACAATACCAAGAACACG
GTGGATCTTCAAATGAACAGCCTGAGAGCCGAGGACACGGCTGTGTATTACTGTGCGAGAGGCTGGTCCTACTTAGACAT
CTGGGGCCAAGGGACCCTGGTCACCGTCTCGAGCGGTGGAGGCGGATCTGGCGGAGGTGGTTCCGGCGGTGGCGGCTCCG
GTGGAGGCGGCTCTGCCCTTGTGATGACCCAGTCTCCATCCTCCCTGTCTGCATCTGTAGGAGACAGAGTCACCATCACT
TGCCAGGCCAGTCAGAACATTTACAGCAATTTAGCCTGGTATCAGCAGAAACCAGGGAAAGTTCCTAAGCTCCTGATCTA
TCAGGCCTCCACTCTGGCATCTGGGGTCCCATCTCGGTTCAGTGGCAGTGGATATGGGACAGATTTCACTCTCACCATCA
GCAGCCTGCAGCCTGAAGATGTTGCAACTTATTACTGTCAAGGCGGTTATTATAGTGCTGCCCTTAATACTTTCGGCGGA
GGGACCAAGGTGGAGATCAAA
SEQ ID 02: CT4 x PD224D1 amino acid sequence (aa)
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLVESGGGLVQPGGSLRLSCTASGFSLSSYA
MSWVRQAPGKGLEYIGYIGDTTGIAYASWANGRFTISKDNTKNTVDLQMNSLRAEDTAVYYCARGWSYLD
IWGQGTLVTVSSGGGGSGGGGSGGGGSGGGGSALVMTQSPSSLSASVGDRVTITCQASQNIYSNLAWYQQ
KPGKVPKLLIYQASTLASGVPSRFSGSGYGTDFTLTISSLQPEDVATYYCQGGYYSAALNTFGGGTKVEI
K
SEQ ID 03: CT4 kappa light chain nt
GAAATTGTGTTGACGCAGTCTCCAGGCACCCTGTCTTTGTCTCCAGGGGAAAGAGCCACCCTCTCCTGCA
GGGCCAGTCAGAGTGTTGGCAGCAGCTACTTAGCCTGGTACCAGCAGAAACCTGGCCAGGCTCCCAGGCT
CCTCATCTATGGTGCATTCAGCAGGGCCACTGGCATCCCAGACAGGTTCAGTGGCAGTGGGTCTGGGACA
GACTTCACTCTCACCATCAGCAGACTGGAGCCTGAAGATTTTGCAGTGTATTACTGTCAGCAGTATGGTA
GCTCACCGTGGACGTTCGGCCAAGGGACCAAGGTGGAAATCAAA
CGTACGGTGGCTGCACCATCTGTCTTCATCTTCCCGCCATCTGATGAGCAGTTGAAATCTGGAACTGCCT
CTGTTGTGTGCCTGCTGAATAACTTCTATCCCAGAGAGGCCAAAGTACAGTGGAAGGTGGATAACGCCCT
CCAATCGGGTAACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGT
SEQ ID 04: CT4 kappa light chain aa
EIVLTQSPGTLSLSPGERATLSCRASQSVGSSYLAWYQQKPGQAPRLLIYGAFSRATGIPDRFSGSGSGT
DFTLTISRLEPEDFAVYYCQQYGSSPWTFGQGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKSF
NRGEC
SEQ ID 05: CT4 x PL23006 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCCCAGTCGGTGGAGGAGTCTGGGGGAGGCTT
GGTCCAGCCTGGGGGGTCCCTGAGACTCTCCTGTACCGCCTCTGGAATCGACCTTAATACCTACGACATG
ATCTGGGTCCGCCAGGCTCCAGGCAAGGGGCTAGAGTGGGTTGGAATCATTACTTATAGTGGTAGTAGAT
ACTACGCGAACTGGGCGAAAGGCCGATTCACCATCTCCAAAGACAATACCAAGAACACGGTGTATCTGCA
AATGAACAGCCTGAGAGCTGAGGACACGGCTGTGTATTACTGTGCGAGAGATTATATGAGTGGTTCCCAC
TTGTGGGGCCAGGGAACCCTGGTCACCGTCTCTTCCGGTGGAGGCGGTTCAGGCGGAGGTGGAAGTGGTG
GTGGCGGCTCTGGAGGCGGCGGATCTGCCTATGATATGACCCAGTCTCCATCTTCCGTGTCTGCATCTGT
AGGAGACAGAGTCACCATCAAGTGTCAGGCCAGTGAGGACATTTATAGCTTCTTGGCCTGGTATCAGCAG
AAACCAGGGAAAGCCCCTAAGCTCCTGATCCATTCTGCATCCTCTCTGGCATCTGGGGTCCCATCAAGGT
TCAGCGGCAGTGGATCTGGGACAGATTTCACTCTCACCATCAGCAGCCTGCAGCCTGAAGATTTTGCAAC
TTACTATTGTCAACAGGGTTATGGTAAAAATAATGTTGATAATGCTTTCGGCGGAGGGACCAAGGTGGAG
ATCAAA
SEQ ID 06: CT4 x PL23006 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSQSVEESGGGLVQPGGSLRLSCTASGIDLNTYDM
IWVRQAPGKGLEWVGIITYSGSRYYANWAKGRFTISKDNTKNTVYLQMNSLRAEDTAVYYCARDYMSGSH
LWGQGTLVTVSSGGGGSGGGGSGGGGSGGGGSAYDMTQSPSSVSASVGDRVTIKCQASEDIYSFLAWYQQ
KPGKAPKLLIHSASSLASGVPSRFSGSGSGTDFTLTISSLQPEDFATYYCQQGYGKNNVDNAFGGGTKVE
IK
SEQ ID 07: CT4 x PL221G5 null2 Fc nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCCGAGGTGCAGCTGTTGGAGTCTGGGGGAGG
CTTGGTACAGCCTGGGGGGTCCCTGAGACTCTCCTGTGCAGCCTCTGGATTCTCCTTCAGTAGCGGGTAC
GACATGTGCTGGGTCCGCCAGGCTCCAGGGAAGGGGCTGGAGTGGATCGCATGCATTGCTGCTGGTAGTG
CTGGTATCACTTACGACGCGAACTGGGCGAAAGGCCGGTTCACCATCTCCAGAGACAATTCCAAGAACAC
GCTGTATCTGCAAATGAACAGCCTGAGAGCCGAGGACACGGCCGTATATTACTGTGCGAGATCGGCGTTT
TCGTTCGACTACGCCATGGACCTCTGGGGCCAGGGAACCCTGGTCACCGTCTCGAGCGGTGGAGGCGGAT
CTGGCGGAGGTGGTTCCGGCGGTGGCGGCTCCGGTGGAGGCGGCTCTGACATCCAGATGACCCAGTCTCC
TTCCACCCTGTCTGCATCTGTAGGAGACAGAGTCACCATCACTTGCCAGGCCAGTCAGAGCATTAGTTCC
CACTTAAACTGGTATCAGCAGAAACCAGGGAAAGCCCCTAAGCTCCTGATCTATAAGGCATCCACTCTGG
CATCTGGGGTCCCATCAAGGTTCAGCGGCAGTGGATCTGGGACAGAATTTACTCTCACCATCAGCAGCCT
GCAGCCTGATGATTTTGCAACTTATTACTGCCAACAGGGTTATAGTTGGGGTAATGTTGATAATGTTTTC
GGCGGAGGGACCAAGGTGGAGATCAAA
SEQ ID 08: CT4 x PL221G5 null Fc aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLLESGGGLVQPGGSLRLSCAASGFSFSSGY
DMCWVRQAPGKGLEWIACIAAGSAGITYDANWAKGRFTISRDNSKNTLYLQMNSLRAEDTAVYYCARSAF
SFDYAMDLWGQGTLVTVSSGGGGSGGGGSGGGGSGGGGSDIQMTQSPSTLSASVGDRVTITCQASQSISS
HLNWYQQKPGKAPKLLIYKASTLASGVPSRFSGSGSGTEFTLTISSLQPDDFATYYCQQGYSWGNVDNVF
GGGTKVEIK
SEQ ID 09: CT4 x PL231H2 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCACAGGTGCAGCTGGTGGAGTCTGGGGGAGG
CCTGGTCCAGCCTGGGGGCTCCCTGAGACTCTCCTGTACAACTTCTGGAATCGACCTTAGTACCTACGAC
ATGATCTGGGTCCGCCAGGCTCCAGGCAAGGGGCTAGAGTGGGTGGGAATCATTAGTTATGTTGGTAACA
CATACTACGCGAGCTGGGCGAAAGGCCGATTCACCCTCTCCAAAGACAATACCTCGACCACGGTGGATCT
GCAAATGAACAGCCTGAGAGCTGAGGACACGGCTGTGTATTACTGTGCGAGAGATTTTATTAGTGGTTCC
CACTTGTGGGGCCAGGGAACCCTGGTCACCGTCTCGAGCGGTGGAGGCGGATCTGGCGGAGGTGGTTCCG
GCGGTGGCGGCTCCGGTGGAGGCGGTTCTGCCTATGATATGACCCAGTCTCCATCTTCCGTGTCTGCATC
TGTAGGAGACAGAGTCACCATCAATTGTCAGGCCAGTGAGAGCATTAGTAGCTTCTTATCCTGGTATCAG
CAGAAACCAGGGAAAGCCCCTAAGCTCCTGATCTTTTCTGCATCCACTCTGGCATCTGGGGTCCCATCAA
GGTTCAGCGGCAGTGGATCTGGGACAGATTTCACTCTCACCATCAGCAGCCTGCAGCCTGAAGATTTTGC
AACTTACTATTGTCAACAGGGTTATAGTAAAAGTAATGTGGATAATGCTTTCGGCGGAGGGACCAAGGTG
GAGATCAAA
SEQ ID 10: CT4 x PL231H2 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSQVQLVESGGGLVQPGGSLRLSCTTSGIDLSTYD
MIWVRQAPGKGLEWVGIISYVGNTYYASWAKGRFTLSKDNTSTTVDLQMNSLRAEDTAVYYCARDFISGS
HLWGQGTLVTVSSGGGGSGGGGSGGGGSGGGGSAYDMTQSPSSVSASVGDRVTINCQASESISSFLSWYQ
QKPGKAPKLLIFSASTLASGVPSRFSGSGSGTDFTLTISSLQPEDFATYYCQQGYSKSNVDNAFGGGTKV
ETK
SEQ ID 11: CT4 x PL004B5 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCAGAGGTGCAGCTGGTGGAGTCTGGGGGAGG
CTTGGTCCAGCCTGGGGGGTCCCTGAGACTCTCCTGTGCAGCCTCTGGATTCTCCCTCAGTAGCTACTGG
ATGAGCTGGGTCCGCCAGGCTCCAGGGAAGGGGCTGGAGTGGATCGGAGTCATTGATACTAATGTTTATA
TATACTACGCGAACTGGGCAAAAGGCAGATTCACCATCTCCAGAGACAATTCCAAGAACACGCTGTATCT
TCAAATGAACAGCCTGAGAGCCGAGGACACGGCTGTGTATTACTGTGCGAGATATGTGGGTAATAATGAT
GATTATATTAACTTGTGGGGCCAGGGAACCCTGGTCACCGTCTCGAGCGGTGGAGGCGGATCTGGCGGAG
GTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGTCCAGTCAGAGTGTTTATAATGGCTACTGG
TTATCCTGGTATCAGCAGAAACCAGGGAAAGCCCCTAAGCTCCTGATCTATGGTGCATCCACTCTGGCAT
CTGGGGTCCCATCAAGGTTCAGCGGCAGTGGATCTGGGACAGAATTCACTCTCACCATCAGCAGCCTGCA
GCCTGATGATTTTGCAACTTATTACTGCCTAGGCAGTTATACTAGTAGTACTGAGAACTCTTTCGGCGGA
GGGACCAAGGTGGAGATCAAA
SEQ ID 12: CT4 x PL004B5 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLVESGGGLVQPGGSLRLSCAASGFSLSSYW
MSWVRQAPGKGLEWIGVIDINVYIYYANWAKGRFTISRDNSKNTLYLQMNSLRAEDTAVYYCARYVGNND
DYINLWGQGTLVTVSSGGGGSGGGGSGGGGSGGGGSDIQMTQSPSTLSASVGDRVTITCQSSQSVYNGYW
LSWYQQKPGKAPKLLIYGASTLASGVPSRFSGSGSGTEFTLTISSLQPDDFATYYCLGSYTSSTENSEGG
GTKVEIK
SEQ ID 13: CT4 x PL004B9 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCAGAGGTGCAGCTGGTGGAGTCTGGGGGAGG
CTTGGTCCAGCCTGGGGGGTCCCTGAGACTCTCCTGTACAGTGTCTGGAATCGACCTCAGTGTCATCAAT
ATGGGCTGGGTCCGCCAGGCTCCAGGGAAGGGGCTGGAGTGGATCGGAACCATTACTTATGTTGGTAACA
CATATTACGCGAGCTGGGCGAAAGGCAGATTCACCATCTCCAAAACCTCGACCACGGTGGATCTTAAAAT
CACCAGTCCGACAACCGAGGACACGGCTGTGTATTACTGTGCGAGAGAATCTGGTACTATTTATTACAGT
TACTTTAACTTGTGGGGCCAAGGGACCCTGGTCACCGTCTCGAGCGGTGGAGGCGGATCTGGCGGAGGTG
GTTCCGGCGGTGGCGGCTCCGGTGGAGGCGGCTCTGCATTCGAATTGACCCAGTCTCCATCCTCCCTGTC
TGCATCTGTAGGAGACAGAGTCACCATCAAGTGCCAGGCCAGTGAAAGCATTAGCAACTACTTATCCTGG
TATCAGCAGATTCCAGGGAAAGTTCCTAAGCTCCTGATCTATTATGCATCCAATCTGGCATCTGGGGTCC
CATCTCGGTTCAGTGGCAGTGGATCTGGGACAGATTTCACTCTCACCATCAGCAGCCTGCAGCCTGAAGA
TGTTGCAACTTATTACTGTCAAAGCTATTATGGTGGTGGTAGTGCCTATACTTTCGGCGGAGGGACCAAG
GTGGAGATCAAA
SEQ ID 14: CT4 x PL004B9 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLVESGGGLVQPGGSLRLSCTVSGIDLSVIN
MGWVRQAPGKGLEWIGTITYVGNTYYASWAKGRFTISKTSTTVDLKITSPTTEDTAVYYCARESGTIYYS
YFNLWGQGTLVTVSSGGGGSGGGGSGGGGSGGGGSAFELTQSPSSLSASVGDRVTIKCQASESISNYLSW
YQQIPGKVPKLLIYYASNLASGVPSRFSGSGSGTDFTLTISSLQPEDVATYYCQSYYGGGSAYTFGGGTK
VEIK
SEQ ID 15: CT4 x PD206F12 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCAGAGGTGCAGCTGGTGGAGTCTGGGGGAGG
CTTGGTCCAGCCTGGGGGGTCCCTGAGACTCTCCTGTGCAGCCTCTGGATTCGACTTCAGTAGCGGCTAC
TGGATATGCTGGGTCCGCCAGGCTCCAGGGAAGGGGCTGGAGTTGATCGCATGCATTTATGCTGGTACTA
GTGGTAGTACTTCCTACGCGAGCTGGGCGAGAGGCAGATTCACCATCTCCGAAACCTCCAAGAACACGGT
GACTCTTCAAATGAACAGCCTGAGAGCCGAGGACTCGGCTGTGTATTACTGTGCGAGAAATCTTTACACT
TACAATAGCTTGTGGGGCCAGGGAACCCTGGTCACCGTCTCGAGCGGTGGAGGCGGATCTGGCGGAGGTG
GTTCCGGCGGTGGCGGCTCCGGTGGAGGCGGCTCTGACATCCAGATGACCCAGTCTCCTTCCACCCTGTC
TGCATCTGTAGGAGACAGAGTCACCATCACTTGCCAGTCCAGTCAGAGTGTTTATGATAACAACTGGTTA
GCCTGGTATCAGCAGAAACCAGGGAAAGCCCCTAAGCTCCTGATCTATACAGTATCCACTCTGGCATCTG
GGGTCCCATCAAGGTTCAGCGGCAGTGGATCTGGGACAGAATTCACTCTCACCATCAGCAGCCTGCAGCC
TGATGATTTTGCAACTTATTACTGCCAAGGCACTTATTATAGTAGTGGTTGGAACTTTGCTTTCGGCGGA
GGGACCAAGGTGGAGATCAAA
SEQ ID 16: CT4 x PD206F12 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLVESGGGLVQPGGSLRLSCAASGFDFSSGY
WICWVRQAPGKGLELIACIYAGTSGSTSYASWARGRFTISETSKNIVTLQMNSLRAEDSAVYYCARNLYT
YNSLWGQGTLVTVSSGGGGSGGGGSGGGGSGGGGSDIQMTQSPSTLSASVGDRVTITCQSSQSVYDNNWL
AWYQQKPGKAPKLLIYTVSTLASGVPSRFSGSGSGTEFTLTISSLQPDDFATYYCQGTYYSSGWNFAFGG
GTKVEIK
SEQ ID 17: CT4 x PD215A1 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCACAGGAGCAGCTGTTGGAGTCTGGGGGAGG
CTTGGTACAGCCTGGGGGGTCCCTGAGACTCTCCTGTACAGCCTCTGGATTCTCCTTTAGCAGCTACTGG
ATGTGCTGGGTCCGCCAGGCTCCAGGGAAGGGGCTGGAGTGGATCGGATGCATTACGACTGGTAGTGGTA
GCACTTACTACGCGAGCTGGGCGAAGCGCCGGTTCACCATCTCCAAAGACAATTCCAAGAACACGGTGAC
TCTGCAAATGAACAGCCTGAGAGCCGAGGACACGGCCGTATATTACTGTACGAGAGCATTTGACTTGTGG
GGCCAGGGAACCCTGGTCACCGTCTCGAGCGGTGGAGGCGGATCTGGCGGAGGTGGTTCCGGCGGTGGCG
GCTCCGGTGGAGGCGGCTCTGACATCCAGATGACCCAGTCTCCATCCTCCCTGTCTGCATCTGTAGGAGA
CAGAGTCACCATCACTTGCCAGGCCAGTCAGAGCATTTACAGCTACTTAAACTGGTATCAGCAGAAACCA
GGGAAAGCCCCTAAGCTCCTGATCTATGGTGCATCCAATCTGGCATCTGGGGTCCCATCAAGGTTCAGTG
GCAGTGGATCTGGGACAGATTTCACTCTCACCATCAGCAGTCTGCAACCTGAAGATTTTGCAACTTACTA
CTGTCAAAGCAGTTGGTTGAGTGGTGCTGTTGGTAATGCTTTCGGCGGAGGGACCAAGGTGGAGATCAAA
SEQ ID 18: CT4 x PD215A1 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSQEQLLESGGGLVQPGGSLRLSCTASGFSFSSYW
MCWVRQAPGKGLEWIGCITTGSGSTYYASWAKRRFTISKDNSKNTVTLQMNSLRAEDTAVYYCTRAFDLW
GQGTLVTVSSGGGGSGGGGSGGGGSGGGGSDIQMTQSPSSLSASVGDRVTITCQASQSIYSYLNWYQQKP
GKAPKLLIYGASNLASGVPSRFSGSGSGTDFTLTISSLQPEDFATYYCQSSWLSGAVGNAFGGGTKVEIK
SEQ ID 19: CT4 x PD220F6 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCACAGGAGCAGGTGAAGGAGACCGGGGGAGG
CTTGGTACAGCCTGGGGGGTCCCTGAGACTCTCCTGTGCAGCCTCTGGATTCACCATCAGCAGCTATGGA
GTGAGCTGGGTCCGCCAGGCTCCAGGGAAGGGGCTGGAGTGGGTCGCATTGATTTTTCCCGGGATTGGTT
TCAAAGACTACGCGAGCTGGGTGAATGGCCGGTTCACCCTCTCCAGCGACAACGCCCAGAACACTGTGGA
ACTGCAAATGAACAGCCTGAGAGCCGAGGACACGGCCGTATATTACTGTGCGAGAGATTTGGACTTGTGG
GGCCAGGGAACCCTGGTCACCGTCTCGAGCGGTGGAGGCGGATCTGGCGGAGGTGGTTCCGGCGGTGGCG
GCTCCGGTGGAGGCGGCTCTGACATCCAGATGACCCAGTCTCCATCCTCCCTGTCTGCATCTGTAGGAGA
CAGAGTCACCATCACTTGCCAGTCCAGTCCGAGTGTTTATAGTAACTACTTATCCTGGTATCAGCAGAAA
CCAGGGAAAGTTCCTAAGCTCCTGATCTATTATGCATCCACTCTGGCATCTGGGGTCCCATCTCGGTTCA
GTGGCAGTGGATCTGGGACAGATTTCACTCTCACCATCAGCAGCCTGCAGCCTGAAGATGTTGCAACTTA
TTACTGTGCAGGCGGTTATAGTAGTAGTACTCGTGCTTTCGGCGGAGGGACCAAGGTGGAGATCAAA
SEQ ID 20: CT4 x PD220F6 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSQEQVKETGGGLVQPGGSLRLSCAASGFTISSYG
VSWVRQAPGKGLEWVALIFPGIGFKDYASWVNGRFTLSSDNAQNTVELQMNSLRAEDTAVYYCARDLDLW
GQGTLVTVSSGGGGSGGGGSGGGGSGGGGSDIQMTQSPSSLSASVGDRVTITCQSSPSVYSNYLSWYQQK
PGKVPKLLIYYASTLASGVPSRFSGSGSGTDFTLTISSLQPEDVATYYCAGGYSSSTRAFGGGTKVEIK
SEQ ID 21: CT4 x PD225G11 nt
CAGGTGCAGCTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
CTCAGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCAGAGGTGCAGCTGTTGGAGTCTGGGGGAGG
CTTGGTACAGCCTGGGGGGTCCCTGAGACTCTCCTGTGCAGCCTCTGGATTCACCTTTAGCAGCAGCCAC
TGGATATGCTGGGTCCGCCAGGCTCCAGGGAAGGGGCTGGAGTGGATCGCATGCATTTATACTGGTAGTA
TTGATGTCTTTTACTACGCGAGCTGGGCGAAAGGCCGGTTCACCATCTCCAGAGACAATTCCAAGAACAC
GCTGTATCTGCAAATGAACAGCCTGAGAGCCGAGGACACGGCCGTATATTACTGTGCGAGAGCCGCTAAT
ACTGATACTACCTACTTTAACTTGTGGGGCCAGGGAACCCTGGTCACCGTCTCGAGCGGTGGAGGCGGAT
CTGGCGGAGGTGGTTCCGGCGGTGGCGGCTCCGGTGGAGGCGGCTCTGCCTATGATATGACCCAGTCTCC
ATCCTCCCTGTCTGCATCTGTAGGAGACAGAGTCACCATCAATTGCCAGGCCAGTCAGAGCATTAACAAC
CAACTATCCTGGTATCAGCAGAAACCAGGGAAAGTTCCTAAGCTCCTGATCTATGGTGCATCCACTCTGG
CATCTGGGGTCCCATCTCGGTTCACCGGCAGTGGATCTGGGACAGATTTCACTCTCACCATCAGCAGCCT
GCAGCCTGAAGATGTTGCAACTTATTACTGTCATGTTCATTATTGCAGTGGTGGTAGTTGTTTTTGGGCT
TTCGGCGGAGGGACCAAGGTGGAGATCAAA
SEQ ID 22: CT4 x PD225G11 aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLLESGGGLVQPGGSLRLSCAASGFTFSSSH
WICWVRQAPGKGLEWIACIYIGSIDVFYYASWAKGRFTISRDNSKNTLYLQMNSLRAEDTAVYYCARAAN
TDTTYFNLWGQGTLVTVSSGGGGSGGGGSGGGGSGGGGSAYDMTQSPSSLSASVGDRVTINCQASQSINN
QLSWYQQKPGKVPKLLIYGASTLASGVPSRFTGSGSGTDFTLTISSLQPEDVATYYCHVHYCSGGSCFWA
FGGGTKVEIK
SEQ ID 23: G4S x 4 nt
GGTGGAGGCGGATCTGGCGGAGGTGGTTCCGGCGGTGGCGGCTCCGGTGGAGGCGGCTCT
SEQ ID 24: G4S x 4 aa
GGGGSGGGGSGGGGSGGGGS
SEQ ID 25: PL23006 x CT4 nt
CAGTCGGTGGAGGAGTCTGGGGGAGGCTTGGTCCAGCCTGGGGGGTCCCTGAGACTCTCCTGTACCGCCT
CTGGAATCGACCTTAATACCTACGACATGATCTGGGTCCGCCAGGCTCCAGGCAAGGGGCTAGAGTGGGT
TGGAATCATTACTTATAGTGGTAGTAGATACTACGCGAACTGGGCGAAAGGCCGATTCACCATCTCCAAA
GACAATACCAAGAACACGGTGTATCTGCAAATGAACAGCCTGAGAGCTGAGGACACGGCTGTGTATTACT
GTGCGAGAGATTATATGAGTGGTTCCCACTTGTGGGGCCAGGGAACCCTGGTCACCGTCTCGAGCGCTAG
CACCAAGGGCCCATCGGTCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCAGCAACACCAAGGTGGAC
AAGAGAGTTGAGCCCAAATCTTGTGACAAAACTCACACATGCCCACCGTGCCCAGCACCTGAAGCCGCGG
GGGCACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGCGTCC
TCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCG
ACATCGCCGTGGAGTGGGAGAGCAATGGGCAGCCGGAGAACAACTACAAGACCACGCCTCCCGTGCTGGA
CTCCGACGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCTCCGG
GTGGCGGTGGAGGGTCCGGCGGTGGTGGATCCCAGGTGCAATTGGTGGAGTCTGGGGGAGGCGTGGTCCA
GCCTGGGAGGTCCCTGAGACTCTCCTGTGCAGCCTCTGGATTCACCTTCAGTAGCTATACTATGCACTGG
GTCCGCCAGGCTCCAGGCAAGGGGCTGGAGTGGGTGACATTTATATCATATGATGGAAACAATAAATACT
ACGCAGACTCCGTGAAGGGCCGATTCACCATCTCCAGAGACAATTCCAAGAACACGCTGTATCTGCAAAT
GAACAGCCTGAGAGCTGAGGACACGGCTATATATTACTGTGCGAGGACCGGCTGGCTGGGGCCCTTTGAC
TACTGGGGCCAGGGAACCCTGGTCACCGTCTCGAGCGGTGGAGGCGGTTCAGGCGGAGGTGGAAGTGGTG
GTGGCGGCTCTGGAGGCGGCGGATCTGAAATTGTGTTGACGCAGTCTCCAGGCACCCTGTCTTTGTCTCC
AGGGGAAAGAGCCACCCTCTCCTGCAGGGCCAGTCAGAGTGTTGGCAGCAGCTACTTAGCCTGGTACCAG
CAGAAACCTGGCCAGGCTCCCAGGCTCCTCATCTATGGTGCATTCAGCAGGGCCACTGGCATCCCAGACA
GGTTCAGTGGCAGTGGGTCTGGGACAGACTTCACTCTCACCATCAGCAGACTGGAGCCTGAAGATTTTGC
AGTGTATTACTGTCAGCAGTATGGTAGCTCACCGTGGACGTTCGGCCAAGGGACCAAGGTGGAAATCAAA
SEQ ID 26: PL23006 x CT4 aa
QSVEESGGGLVQPGGSLRLSCTASGIDLNTYDMIWVRQAPGKGLEWVGIITYSGSRYYANWAKGRFTISK
DNIKNITYLQMNSLRAEDTAVYYCARDYMSGSHLWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVD
KRVEPKSCDKTHTCPPCPAPEAAGAPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSQVQLVESGGGVVQPGRSLRLSCAASGFTFSSYTMHW
VRQAPGKGLEWVTFISYDGNNKYYADSVKGRFTISRDNSKNTLYLQMNSLRAEDTAIYYCARTGWLGPFD
YWGQGTLVTVSSGGGGSGGGGSGGGGSGGGGSEIVLTQSPGTLSLSPGERATLSCRASQSVGSSYLAWYQ
QKPGQAPRLLIYGAFSRATGIPDRFSGSGSGTDFTLTISRLEPEDFAVYYCQQYGSSPWTFGQGTKVEIK
SEQ ID 27: PL23006 kappa light chain nt
GCCTATGATATGACCCAGTCTCCATCTTCCGTGTCTGCATCTGTAGGAGACAGAGTCACCATCAAGTGTC
AGGCCAGTGAGGACATTTATAGCTTCTTGGCCTGGTATCAGCAGAAACCAGGGAAAGCCCCTAAGCTCCT
GATCCATTCTGCATCCTCTCTGGCATCTGGGGTCCCATCAAGGTTCAGCGGCAGTGGATCTGGGACAGAT
TTCACTCTCACCATCAGCAGCCTGCAGCCTGAAGATTTTGCAACTTACTATTGTCAACAGGGTTATGGTA
AAAATAATGTTGATAATGCTTTCGGCGGAGGGACCAAGGTGGAGATCAAA
CGTACGGTGGCTGCACCATCTGTCTTCATCTTCCCGCCATCTGATGAGCAGTTGAAATCTGGAACTGCCT
CTGTTGTGTGCCTGCTGAATAACTTCTATCCCAGAGAGGCCAAAGTACAGTGGAAGGTGGATAACGCCCT
CCAATCGGGTAACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGT
SEQ ID 28: PL23006 kappa light chain aa
AYDMTQSPSSVSASVGDRVTIKCQASEDIYSFLAWYQQKPGKAPKLLIHSASSLASGVPSRFSGSGSGTD
FTLTISSLQPEDFATYYCQQGYGKNNVDNAFGGGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTK
SFNRGEC
SEQ ID 29: PD224D1 x CT4 nt
GAGGTGCAGCTGGTGGAGTCTGGGGGAGGCTTGGTCCAGCCTGGGGGGTCCCTGAGACTCTCCTGTACAG
CCTCTGGATTCTCCCTCAGTAGCTATGCAATGAGCTGGGTCCGCCAGGCTCCAGGGAAGGGGCTGGAGTA
CATCGGCTACATTGGTGATACTACTGGCATAGCCTACGCGAGCTGGGCGAATGGCAGATTCACCATCTCC
AAAGACAATACCAAGAACACGGTGGATCTTCAAATGAACAGCCTGAGAGCCGAGGACACGGCTGTGTATT
ACTGTGCGAGAGGCTGGTCCTACTTAGACATCTGGGGCCAAGGGACCCTGGTCACCGTCTCGAGCGCTAG
CACCAAGGGCCCATCGGTCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCAGCAACACCAAGGTGGAC
AAGAGAGTTGAGCCCAAATCTTGTGACAAAACTCACACATGCCCACCGTGCCCAGCACCTGAAGCCGCGG
GGGCACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGCGTCC
TCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCG
ACATCGCCGTGGAGTGGGAGAGCAATGGGCAGCCGGAGAACAACTACAAGACCACGCCTCCCGTGCTGGA
CTCCGACGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCTCCGG
GTGGCGGTGGAGGGTCCGGCGGTGGTGGATCCCAGGTGCAATTGGTGGAGTCTGGGGGAGGCGTGGTCCA
GCCTGGGAGGTCCCTGAGACTCTCCTGTGCAGCCTCTGGATTCACCTTCAGTAGCTATACTATGCACTGG
GTCCGCCAGGCTCCAGGCAAGGGGCTGGAGTGGGTGACATTTATATCATATGATGGAAACAATAAATACT
ACGCAGACTCCGTGAAGGGCCGATTCACCATCTCCAGAGACAATTCCAAGAACACGCTGTATCTGCAAAT
GAACAGCCTGAGAGCTGAGGACACGGCTATATATTACTGTGCGAGGACCGGCTGGCTGGGGCCCTTTGAC
TACTGGGGCCAGGGAACCCTGGTCACCGTCTCGAGCGGTGGAGGCGGATCTGGCGGAGGTGGTTCCGGCG
GTGGCGGCTCCGGTGGAGGCGGTTCTGAAATTGTGTTGACGCAGTCTCCAGGCACCCTGTCTTTGTCTCC
AGGGGAAAGAGCCACCCTCTCCTGCAGGGCCAGTCAGAGTGTTGGCAGCAGCTACTTAGCCTGGTACCAG
CAGAAACCTGGCCAGGCTCCCAGGCTCCTCATCTATGGTGCATTCAGCAGGGCCACTGGCATCCCAGACA
GGTTCAGTGGCAGTGGGTCTGGGACAGACTTCACTCTCACCATCAGCAGACTGGAGCCTGAAGATTTTGC
AGTGTATTACTGTCAGCAGTATGGTAGCTCACCGTGGACGTTCGGCCAAGGGACCAAGGTGGAAATCAAA
SEQ ID 30: PD224D1 x CT4 aa
EVQLVESGGGLVQPGGSLRLSCIASGESLSSYAMSWVRQAPGKGLEYIGYIGDTTGIAYASWANGRFTIS
KDNTKNTVDLQMNSLRAEDTAVYYCARGWSYLDIWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVD
KRVEPKSCDKTHTCPPCPAPEAAGAPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSQVQLVESGGGVVQPGRSLRLSCAASGFTFSSYTMHW
VRQAPGKGLEWVTFISYDGNNKYYADSVKGRFTISRDNSKNTLYLQMNSLRAEDTAIYYCARTGWLGPFD
YWGQGTLVTVSSGGGGSGGGGSGGGGSGGGGSEIVLTQSPGTLSLSPGERATLSCRASQSVGSSYLAWYQ
QKPGQAPRLLIYGAFSRATGIPDRFSGSGSGTDFTLTISRLEPEDFAVYYCQQYGSSPWTFGQGTKVEIK
SEQ ID 31: PD224 light chain nt
GCCCTTGTGATGACCCAGTCTCCATCCTCCCTGTCTGCATCTGTAGGAGACAGAGTCACCATCACTTGCC
AGGCCAGTCAGAACATTTACAGCAATTTAGCCTGGTATCAGCAGAAACCAGGGAAAGTTCCTAAGCTCCT
GATCTATCAGGCCTCCACTCTGGCATCTGGGGTCCCATCTCGGTTCAGTGGCAGTGGATATGGGACAGAT
TTCACTCTCACCATCAGCAGCCTGCAGCCTGAAGATGTTGCAACTTATTACTGTCAAGGCGGTTATTATA
GTGCTGCCCTTAATACTTTCGGCGGAGGGACCAAGGTGGAGATCAAA
CGTACGGTGGCTGCACCATCTGTCTTCATCTTCCCGCCATCTGATGAGCAGTTGAAATCTGGAACTGCCT
CTGTTGTGTGCCTGCTGAATAACTTCTATCCCAGAGAGGCCAAAGTACAGTGGAAGGTGGATAACGCCCT
CCAATCGGGTAACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGT
SEQ ID 32: PD224 light chain aa
ALVMTQSPSSLSASVGDRVTITCQASQNIYSNLAWYQQKPGKVPKLLIYQASTLASGVPSRFSGSGYGTD
FTLTISSLQPEDVATYYCQGGYYSAALNTFGGGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTKS
FNRGEC
SEQ ID 33: PL221G5 x CT4 null Fc nt
GAGGTGCAGCTGTTGGAGTCTGGGGGAGGCTTGGTACAGCCTGGGGGGTCCCTGAGACTCTCCTGTGCAG
CCTCTGGATTCTCCTTCAGTAGCGGGTACGACATGTGCTGGGTCCGCCAGGCTCCAGGGAAGGGGCTGGA
GTGGATCGCATGCATTGCTGCTGGTAGTGCTGGTATCACTTACGACGCGAACTGGGCGAAAGGCCGGTTC
ACCATCTCCAGAGACAATTCCAAGAACACGCTGTATCTGCAAATGAACAGCCTGAGAGCCGAGGACACGG
CCGTATATTACTGTGCGAGATCGGCGTTTTCGTTCGACTACGCCATGGACCTCTGGGGCCAGGGAACCCT
GGTCACCGTCTCGAGCGCTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAACTCACACATGCCCACCGT
GCCCAGCACCTGAAGCCGCGGGGGCACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGCAAGGAGTACAAGTGCGC
GGTCTCCAACAAAGCCCTCCCAGCCCCCATCGAGAAAACCATCTCCAAAGCCAAAGGGCAGCCCCGAGAA
CCACAGGTGTACACCCTGCCCCCATCCCGGGATGAGCTGACCAAGAACCAGGTCAGCCTGACCTGCCTGG
TCAAAGGCTTCTATCCCAGCGACATCGCCGTGGAGTGGGAGAGCAATGGGCAGCCGGAGAACAACTACAA
GACCACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACGCAGA
AGAGCCTCTCCCTGTCTCCGGGTGGCGGTGGAGGGTCCGGCGGTGGTGGATCCCAGGTGCAATTGGTGGA
GTCTGGGGGAGGCGTGGTCCAGCCTGGGAGGTCCCTGAGACTCTCCTGTGCAGCCTCTGGATTCACCTTC
AGTAGCTATACTATGCACTGGGTCCGCCAGGCTCCAGGCAAGGGGCTGGAGTGGGTGACATTTATATCAT
ATGATGGAAACAATAAATACTACGCAGACTCCGTGAAGGGCCGATTCACCATCTCCAGAGACAATTCCAA
GAACACGCTGTATCTGCAAATGAACAGCCTGAGAGCTGAGGACACGGCTATATATTACTGTGCGAGGACC
GGCTGGCTGGGGCCCTTTGACTACTGGGGCCAGGGAACCCTGGTCACCGTCTCGAGCGGTGGAGGCGGAT
CTGGCGGAGGTGGTTCCGGCGGTGGCGGCTCCGGTGGAGGCGGTTCTGAAATTGTGTTGACGCAGTCTCC
AGGCACCCTGTCTTTGTCTCCAGGGGAAAGAGCCACCCTCTCCTGCAGGGCCAGTCAGAGTGTTGGCAGC
AGCTACTTAGCCTGGTACCAGCAGAAACCTGGCCAGGCTCCCAGGCTCCTCATCTATGGTGCATTCAGCA
GGGCCACTGGCATCCCAGACAGGTTCAGTGGCAGTGGGTCTGGGACAGACTTCACTCTCACCATCAGCAG
ACTGGAGCCTGAAGATTTTGCAGTGTATTACTGTCAGCAGTATGGTAGCTCACCGTGGACGTTCGGCCAA
GGGACCAAGGTGGAAATCAAA
SEQ ID 34: PL221G5 x CT4 null Fc aa
EVQLLESGGGLVQPGGSLRLSCAASGFSFSSGYDMCWVRQAPGKGLEWIACIAAGSAGITYDANWAKGRF
TISRDNSKNTLYLQMNSLRAEDTAVYYCARSAFSFDYAMDLWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHK
PSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPRE
PQVYTLPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGGGGGSGGGGSQVQLVESGGGVVQPGRSLRLSCAASGFTF
SSYTMHWVRQAPGKGLEWVTFISYDGNNKYYADSVKGRFTISRDNSKNTLYLQMNSLRAEDTAIYYCART
GWLGPFDYWGQGTLVTVSSGGGGSGGGGSGGGGSGGGGSEIVLTQSPGTLSLSPGERATLSCRASQSVGS
SYLAWYQQKPGQAPRLLIYGAFSRATGIPDRFSGSGSGTDFTLTISRLEPEDFAVYYCQQYGSSPWTFGQ
GTKVEIK
SEQ ID 35: PL221G5 Light Chain nt
GACATCCAGATGACCCAGTCTCCTTCCACCCTGTCTGCATCTGTAGGAGACAGAGTCACCATCACTTGCC
AGGCCAGTCAGAGCATTAGTTCCCACTTAAACTGGTATCAGCAGAAACCAGGGAAAGCCCCTAAGCTCCT
GATCTATAAGGCATCCACTCTGGCATCTGGGGTCCCATCAAGGTTCAGCGGCAGTGGATCTGGGACAGAA
TTTACTCTCACCATCAGCAGCCTGCAGCCTGATGATTTTGCAACTTATTACTGCCAACAGGGTTATAGTT
GGGGTAATGTTGATAATGTTTTCGGCGGAGGGACCAAGGTGGAGATCAAACGTACGGTGGCTGCACCATC
TGTCTTCATCTTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGTAACTCCCAGG
AGAGTGTCACAGAGCAGGACAGCAAGGACAGCACCTACAGCCTCAGCAGCACCCTGACGCTGAGCAAAGC
AGACTACGAGAAACACAAAGTCTACGCCTGCGAAGTCACCCATCAGGGCCTGAGCTCGCCCGTCACAAAG
AGCTTCAACAGGGGAGAGTGT
SEQ ID 36: PL221G5 Light Chain aa
DIQMTQSPSTLSASVGDRVTITCQASQSISSHLNWYQQKPGKAPKLLIYKASTLASGVPSRFSGSGSGTE
FTLTISSLQPDDFATYYCQQGYSWGNVDNVEGGGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTK
SFNRGEC
SEQ ID 37: PL231H2 x CT4 null Fc nt
CAGGTGCAGCTGGTGGAGTCTGGGGGAGGCCTGGTCCAGCCTGGGGGCTCCCTGAGACTCTCCTGTACAA
CTTCTGGAATCGACCTTAGTACCTACGACATGATCTGGGTCCGCCAGGCTCCAGGCAAGGGGCTAGAGTG
GGTGGGAATCATTAGTTATGTTGGTAACACATACTACGCGAGCTGGGCGAAAGGCCGATTCACCCTCTCC
AAAGACAATACCTCGACCACGGTGGATCTGCAAATGAACAGCCTGAGAGCTGAGGACACGGCTGTGTATT
ACTGTGCGAGAGATTTTATTAGTGGTTCCCACTTGTGGGGCCAGGGAACCCTGGTCACCGTCTCGAGCGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAAGCCG
CGGGGGCACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCGCGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCCAGGTGCAATTGGTGGAGTCTGGGGGAGGCGTGGT
CCAGCCTGGGAGGTCCCTGAGACTCTCCTGTGCAGCCTCTGGATTCACCTTCAGTAGCTATACTATGCAC
TGGGTCCGCCAGGCTCCAGGCAAGGGGCTGGAGTGGGTGACATTTATATCATATGATGGAAACAATAAAT
ACTACGCAGACTCCGTGAAGGGCCGATTCACCATCTCCAGAGACAATTCCAAGAACACGCTGTATCTGCA
AATGAACAGCCTGAGAGCTGAGGACACGGCTATATATTACTGTGCGAGGACCGGCTGGCTGGGGCCCTTT
GACTACTGGGGCCAGGGAACCCTGGTCACCGTCTCGAGCGGTGGAGGCGGATCTGGCGGAGGTGGTTCCG
GCGGTGGCGGCTCCGGTGGAGGCGGTTCTGAAATTGTGTTGACGCAGTCTCCAGGCACCCTGTCTTTGTC
TCCAGGGGAAAGAGCCACCCTCTCCTGCAGGGCCAGTCAGAGTGTTGGCAGCAGCTACTTAGCCTGGTAC
CAGCAGAAACCTGGCCAGGCTCCCAGGCTCCTCATCTATGGTGCATTCAGCAGGGCCACTGGCATCCCAG
ACAGGTTCAGTGGCAGTGGGTCTGGGACAGACTTCACTCTCACCATCAGCAGACTGGAGCCTGAAGATTT
TGCAGTGTATTACTGTCAGCAGTATGGTAGCTCACCGTGGACGTTCGGCCAAGGGACCAAGGTGGAAATC
AAA
SEQ ID 38: PL231H2 x CT4 null Fc aa
QVQLVESGGGLVQPGGSLRLSCTTSGIDLSTYDMIWVRQAPGKGLEWVGIISYVGNTYYASWAKGRFTLS
KDNTSTTVDLQMNSLRAEDTAVYYCARDFISGSHLWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPEAAGAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSQVQLVESGGGVVQPGRSLRLSCAASGFTFSSYTMH
WVRQAPGKGLEWVTFISYDGNNKYYADSVKGRFTISRDNSKNTLYLQMNSLRAEDTAIYYCARTGWLGPF
DYWGQGTLVTVSSGGGGSGGGGSGGGGSGGGGSEIVLTQSPGTLSLSPGERATLSCRASQSVGSSYLAWY
QQKPGQAPRLLIYGAFSRATGIPDRFSGSGSGTDFTLTISRLEPEDFAVYYCQQYGSSPWTFGQGTKVEI
K
SEQ ID 39: PL231H2 Light Chain nt
GCCTATGATATGACCCAGTCTCCATCTTCCGTGTCTGCATCTGTAGGAGACAGAGTCACCATCAATTGTC
AGGCCAGTGAGAGCATTAGTAGCTTCTTATCCTGGTATCAGCAGAAACCAGGGAAAGCCCCTAAGCTCCT
GATCTTTTCTGCATCCACTCTGGCATCTGGGGTCCCATCAAGGTTCAGCGGCAGTGGATCTGGGACAGAT
TTCACTCTCACCATCAGCAGCCTGCAGCCTGAAGATTTTGCAACTTACTATTGTCAACAGGGTTATAGTA
AAAGTAATGTGGATAATGCTTTCGGCGGAGGGACCAAGGTGGAGATCAAACGTACGGTGGCTGCACCATC
TGTCTTCATCTTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGTAACTCCCAGG
AGAGTGTCACAGAGCAGGACAGCAAGGACAGCACCTACAGCCTCAGCAGCACCCTGACGCTGAGCAAAGC
AGACTACGAGAAACACAAAGTCTACGCCTGCGAAGTCACCCATCAGGGCCTGAGCTCGCCCGTCACAAAG
AGCTTCAACAGGGGAGAGTGT
SEQ ID 40: PL231H2 Light Chain aa
AYDMTQSPSSVSASVGDRVTINCQASESISSFLSWYQQKPGKAPKLLIFSASTLASGVPSRFSGSGSGTD
FTLTISSLQPEDFATYYCQQGYSKSNVDNAFGGGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTK
SFNRGEC
SEQ ID 41: PL004B5 x CT4 nt
GAGGTGCAGCTGGTGGAGTCTGGGGGAGGCTTGGTCCAGCCTGGGGGGTCCCTGAGACTCTCCTGTGCAG
CCTCTGGATTCTCCCTCAGTAGCTACTGGATGAGCTGGGTCCGCCAGGCTCCAGGGAAGGGGCTGGAGTG
GATCGGAGTCATTGATACTAATGTTTATATATACTACGCGAACTGGGCAAAAGGCAGATTCACCATCTCC
AGAGACAATTCCAAGAACACGCTGTATCTTCAAATGAACAGCCTGAGAGCCGAGGACACGGCTGTGTATT
ACTGTGCGAGATATGTGGGTAATAATGATGATTATATTAACTTGTGGGGCCAGGGAACCCTGGTCACCGT
CTCGAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAAGCCGCGGGGGCACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCGCGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGCGGTGGAGGGTCCGGCGGTGGTGGATCCCAGGTGCAATTGGTGGAGTCTGGGGG
AGGCGTGGTCCAGCCTGGGAGGTCCCTGAGACTCTCCTGTGCAGCCTCTGGATTCACCTTCAGTAGCTAT
ACTATGCACTGGGTCCGCCAGGCTCCAGGCAAGGGGCTGGAGTGGGTGACATTTATATCATATGATGGAA
ACAATAAATACTACGCAGACTCCGTGAAGGGCCGATTCACCATCTCCAGAGACAATTCCAAGAACACGCT
GTATCTGCAAATGAACAGCCTGAGAGCTGAGGACACGGCTATATATTACTGTGCGAGGACCGGCTGGCTG
GGGCCCTTTGACTACTGGGGCCAGGGAACCCTGGTCACCGTCTCGAGCGGTGGAGGCGGATCTGGCGGAG
GTGGTTCCGGCGGTGGCGGCTCCGGTGGAGGCGGTTCTGAAATTGTGTTGACGCAGTCTCCAGGCACCCT
GTCTTTGTCTCCAGGGGAAAGAGCCACCCTCTCCTGCAGGGCCAGTCAGAGTGTTGGCAGCAGCTACTTA
GCCTGGTACCAGCAGAAACCTGGCCAGGCTCCCAGGCTCCTCATCTATGGTGCATTCAGCAGGGCCACTG
GCATCCCAGACAGGTTCAGTGGCAGTGGGTCTGGGACAGACTTCACTCTCACCATCAGCAGACTGGAGCC
TGAAGATTTTGCAGTGTATTACTGTCAGCAGTATGGTAGCTCACCGTGGACGTTCGGCCAAGGGACCAAG
GTGGAAATCAAA
SEQ ID 42: PL004B5 x CT4 aa
EVQLVESGGGLVQPGGSLRLSCAASGFSLSSYWMSWVRQAPGKGLEWIGVIDTNVYIYYANWAKGRFTIS
RDNSKNTLYLQMNSLRAEDTAVYYCARYVGNNDDYINLWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPEAAGAPSVFLFPPKPKDTLMISRTPEVTCVVVDVSHEDPEVKFNWY
VDGVEVHNAKTKPREEQYNSTYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGSGGGGSQVQLVESGGGVVQPGRSLRLSCAASGFTFSSY
TMHWVRQAPGKGLEWVTFISYDGNNKYYADSVKGRFTISRDNSKNTLYLQMNSLRAEDTAIYYCARTGWL
GPFDYWGQGTLVTVSSGGGGSGGGGSGGGGSGGGGSEIVLTQSPGTLSLSPGERATLSCRASQSVGSSYL
AWYQQKPGQAPRLLIYGAFSRATGIPDRFSGSGSGTDFTLTISRLEPEDFAVYYCQQYGSSPWTFGQGTK
VEIK
SEQ ID 43: PL004B5 Light Chain nt
GACATCCAGATGACCCAGTCTCCTTCCACCCTGTCTGCATCTGTAGGAGACAGAGTCACCATCACTTGCC
AGTCCAGTCAGAGTGTTTATAATGGCTACTGGTTATCCTGGTATCAGCAGAAACCAGGGAAAGCCCCTAA
GCTCCTGATCTATGGTGCATCCACTCTGGCATCTGGGGTCCCATCAAGGTTCAGCGGCAGTGGATCTGGG
ACAGAATTCACTCTCACCATCAGCAGCCTGCAGCCTGATGATTTTGCAACTTATTACTGCCTAGGCAGTT
ATACTAGTAGTACTGAGAACTCTTTCGGCGGAGGGACCAAGGTGGAGATCAAACGTACGGTGGCTGCACC
ATCTGTCTTCATCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAGCACCCTGACGCTGAGCAA
AGCAGACTACGAGAAACACAAAGTCTACGCCTGCGAAGTCACCCATCAGGGCCTGAGCTCGCCCGTCACA
AAGAGCTTCAACAGGGGAGAGTGT
SEQ ID 44: PL004B5 Light Chain aa
DIQMTQSPSTLSASVGDRVTITCQSSQSVYNGYWLSWYQQKPGKAPKLLIYGASTLASGVPSRFSGSGSG
TEFTLTISSLQPDDFATYYCLGSYTSSTENSFGGGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSPVT
KSFNRGEC
SEQ ID 45: PL004B9 x CT4 nt
GAGGTGCAGCTGGTGGAGTCTGGGGGAGGCTTGGTCCAGCCTGGGGGGTCCCTGAGACTCTCCTGTACAG
TGTCTGGAATCGACCTCAGTGTCATCAATATGGGCTGGGTCCGCCAGGCTCCAGGGAAGGGGCTGGAGTG
GATCGGAACCATTACTTATGTTGGTAACACATATTACGCGAGCTGGGCGAAAGGCAGATTCACCATCTCC
AAAACCTCGACCACGGTGGATCTTAAAATCACCAGTCCGACAACCGAGGACACGGCTGTGTATTACTGTG
CGAGAGAATCTGGTACTATTTATTACAGTTACTTTAACTTGTGGGGCCAAGGGACCCTGGTCACCGTCTC
GAGCGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCCCAGGTGCAATTGGTGGAGTCTGGGGGAGG
CGTGGTCCAGCCTGGGAGGTCCCTGAGACTCTCCTGTGCAGCCTCTGGATTCACCTTCAGTAGCTATACT
ATGCACTGGGTCCGCCAGGCTCCAGGCAAGGGGCTGGAGTGGGTGACATTTATATCATATGATGGAAACA
ATAAATACTACGCAGACTCCGTGAAGGGCCGATTCACCATCTCCAGAGACAATTCCAAGAACACGCTGTA
TCTGCAAATGAACAGCCTGAGAGCTGAGGACACGGCTATATATTACTGTGCGAGGACCGGCTGGCTGGGG
CCCTTTGACTACTGGGGCCAGGGAACCCTGGTCACCGTCTCGAGCGGTGGAGGCGGATCTGGCGGAGGTG
GTTCCGGCGGTGGCGGCTCCGGTGGAGGCGGTTCTGAAATTGTGTTGACGCAGTCTCCAGGCACCCTGTC
TTTGTCTCCAGGGGAAAGAGCCACCCTCTCCTGCAGGGCCAGTCAGAGTGTTGGCAGCAGCTACTTAGCC
TGGTACCAGCAGAAACCTGGCCAGGCTCCCAGGCTCCTCATCTATGGTGCATTCAGCAGGGCCACTGGCA
TCCCAGACAGGTTCAGTGGCAGTGGGTCTGGGACAGACTTCACTCTCACCATCAGCAGACTGGAGCCTGA
AGATTTTGCAGTGTATTACTGTCAGCAGTATGGTAGCTCACCGTGGACGTTCGGCCAAGGGACCAAGGTG
GAAATCAAA
SEQ ID 46: PL004B9 x CT4 aa
EVQLVESGGGLVQPGGSLRLSCTVSGIDLSVINMGWVRQAPGKGLEWIGTITYVGNTYYASWAKGRFTIS
KTSTTVDLKITSPTTEDTAVYYCARESGTIYYSYFNLWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSQVQLVESGGGVVQPGRSLRLSCAASGFTFSSYT
MHWVRQAPGKGLEWVTFISYDGNNKYYADSVKGRFTISRDNSKNTLYLQMNSLRAEDTAIYYCARTGWLG
PFDYWGQGTLVTVSSGGGGSGGGGSGGGGSGGGGSEIVLTQSPGTLSLSPGERATLSCRASQSVGSSYLA
WYQQKPGQAPRLLIYGAFSRATGIPDRFSGSGSGTDFTLTISRLEPEDFAVYYCQQYGSSPWTFGQGTKV
EIK
SEQ ID 47: PL004B9 Light Chain nt
GCATTCGAATTGACCCAGTCTCCATCCTCCCTGTCTGCATCTGTAGGAGACAGAGTCACCATCAAGTGCC
AGGCCAGTGAAAGCATTAGCAACTACTTATCCTGGTATCAGCAGATTCCAGGGAAAGTTCCTAAGCTCCT
GATCTATTATGCATCCAATCTGGCATCTGGGGTCCCATCTCGGTTCAGTGGCAGTGGATCTGGGACAGAT
TTCACTCTCACCATCAGCAGCCTGCAGCCTGAAGATGTTGCAACTTATTACTGTCAAAGCTATTATGGTG
GTGGTAGTGCCTATACTTTCGGCGGAGGGACCAAGGTGGAGATCAAACGTACGGTGGCTGCACCATCTGT
CTTCATCTTCCCGCCATCTGATGAGCAGTTGAAATCTGGAACTGCCTCTGTTGTGTGCCTGCTGAATAAC
TTCTATCCCAGAGAGGCCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGACGCTGAGCAAAGCAGA
CTACGAGAAACACAAAGTCTACGCCTGCGAAGTCACCCATCAGGGCCTGAGCTCGCCCGTCACAAAGAGC
TTCAACAGGGGAGAGTGT
SEQ ID 48: PL004B9 Light Chain aa
AFELTQSPSSLSASVGDRVTIKCQASESISNYLSWYQQIPGKVPKLLIYYASNLASGVPSRFSGSGSGTD
FTLTISSLQPEDVATYYCQSYYGGGSAYTFGGGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTKS
FNRGEC
SEQ ID 49: PD206F12 x CT4 nt
GAGGTGCAGCTGGTGGAGTCTGGGGGAGGCTTGGTCCAGCCTGGGGGGTCCCTGAGACTCTCCTGTGCAG
CCTCTGGATTCGACTTCAGTAGCGGCTACTGGATATGCTGGGTCCGCCAGGCTCCAGGGAAGGGGCTGGA
GTTGATCGCATGCATTTATGCTGGTACTAGTGGTAGTACTTCCTACGCGAGCTGGGCGAGAGGCAGATTC
ACCATCTCCGAAACCTCCAAGAACACGGTGACTCTTCAAATGAACAGCCTGAGAGCCGAGGACTCGGCTG
TGTATTACTGTGCGAGAAATCTTTACACTTACAATAGCTTGTGGGGCCAGGGAACCCTGGTCACCGTCTC
GAGCGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTG
AAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCC
TGTCTCCGGGTGGCGGTGGAGGGTCCGGCGGTGGTGGATCCCAGGTGCAATTGGTGGAGTCTGGGGGAGG
CGTGGTCCAGCCTGGGAGGTCCCTGAGACTCTCCTGTGCAGCCTCTGGATTCACCTTCAGTAGCTATACT
ATGCACTGGGTCCGCCAGGCTCCAGGCAAGGGGCTGGAGTGGGTGACATTTATATCATATGATGGAAACA
ATAAATACTACGCAGACTCCGTGAAGGGCCGATTCACCATCTCCAGAGACAATTCCAAGAACACGCTGTA
TCTGCAAATGAACAGCCTGAGAGCTGAGGACACGGCTATATATTACTGTGCGAGGACCGGCTGGCTGGGG
CCCTTTGACTACTGGGGCCAGGGAACCCTGGTCACCGTCTCGAGCGGTGGAGGCGGATCTGGCGGAGGTG
GTTCCGGCGGTGGCGGCTCCGGTGGAGGCGGTTCTGAAATTGTGTTGACGCAGTCTCCAGGCACCCTGTC
TTTGTCTCCAGGGGAAAGAGCCACCCTCTCCTGCAGGGCCAGTCAGAGTGTTGGCAGCAGCTACTTAGCC
TGGTACCAGCAGAAACCTGGCCAGGCTCCCAGGCTCCTCATCTATGGTGCATTCAGCAGGGCCACTGGCA
TCCCAGACAGGTTCAGTGGCAGTGGGTCTGGGACAGACTTCACTCTCACCATCAGCAGACTGGAGCCTGA
AGATTTTGCAGTGTATTACTGTCAGCAGTATGGTAGCTCACCGTGGACGTTCGGCCAAGGGACCAAGGTG
GAAATCAAA
SEQ ID 50: PD206F12 x CT4 aa
EVQLVESGGGLVQPGGSLRLSCAASGFDFSSGYWICWVRQAPGKGLELIACIYAGTSGSTSYASWARGRF
TISETSKNTVTLQMNSLRAEDSAVYYCARNLYTYNSLWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSQVQLVESGGGVVQPGRSLRLSCAASGFTFSSYT
MHWVRQAPGKGLEWVTFISYDGNNKYYADSVKGRFTISRDNSKNTLYLQMNSLRAEDTAIYYCARTGWLG
PFDYWGQGTLVTVSSGGGGSGGGGSGGGGSGGGGSEIVLTQSPGTLSLSPGERATLSCRASQSVGSSYLA
WYQQKPGQAPRLLIYGAFSRATGIPDRFSGSGSGTDFTLTISRLEPEDFAVYYCQQYGSSPWTFGQGTKV
EIK
SEQ ID 51: PD206F12 Light Chain nt
GACATCCAGATGACCCAGTCTCCTTCCACCCTGTCTGCATCTGTAGGAGACAGAGTCACCATCACTTGCC
AGTCCAGTCAGAGTGTTTATGATAACAACTGGTTAGCCTGGTATCAGCAGAAACCAGGGAAAGCCCCTAA
GCTCCTGATCTATACAGTATCCACTCTGGCATCTGGGGTCCCATCAAGGTTCAGCGGCAGTGGATCTGGG
ACAGAATTCACTCTCACCATCAGCAGCCTGCAGCCTGATGATTTTGCAACTTATTACTGCCAAGGCACTT
ATTATAGTAGTGGTTGGAACTTTGCTTTCGGCGGAGGGACCAAGGTGGAGATCAAACGTACGGTGGCTGC
ACCATCTGTCTTCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAGCACCCTGACGCTGAG
CAAAGCAGACTACGAGAAACACAAAGTCTACGCCTGCGAAGTCACCCATCAGGGCCTGAGCTCGCCCGTC
ACAAAGAGCTTCAACAGGGGAGAGTGT
SEQ ID 52: PD206F12 Light Chain aa
DIQMTQSPSTLSASVGDRVTITCQSSQSVYDNNWLAWYQQKPGKAPKLLIYTVSTLASGVPSRFSGSGSG
TEFTLTISSLQPDDFATYYCQGTYYSSGWNFAFGGGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
SEQ ID 53: PD215A1 x CT4 nt
CAGGAGCAGCTGTTGGAGTCTGGGGGAGGCTTGGTACAGCCTGGGGGGTCCCTGAGACTCTCCTGTACAG
CCTCTGGATTCTCCTTTAGCAGCTACTGGATGTGCTGGGTCCGCCAGGCTCCAGGGAAGGGGCTGGAGTG
GATCGGATGCATTACGACTGGTAGTGGTAGCACTTACTACGCGAGCTGGGCGAAGCGCCGGTTCACCATC
TCCAAAGACAATTCCAAGAACACGGTGACTCTGCAAATGAACAGCCTGAGAGCCGAGGACACGGCCGTAT
ATTACTGTACGAGAGCATTTGACTTGTGGGGCCAGGGAACCCTGGTCACCGTCTCGAGCGCTAGCACCAA
GGGCCCATCGGTCTTCCCCCTGGCACCCTCCTCCAAGAGCACCTCTGGGGGCACAGCGGCCCTGGGCTGC
CTGGTCAAGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCCCTCCAG
CAGCTTGGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAAGCCGCGGGGGCAC
CGTCAGTCTTCCTCTTCCCCCCAAAACCCAAGGACACCCTCATGATCTCCCGGACCCCTGAGGTCACATG
CGTGGTGGTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTCCTCACCG
TCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGACTCCGA
CGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCG
GTGGAGGGTCCGGCGGTGGTGGATCCCAGGTGCAATTGGTGGAGTCTGGGGGAGGCGTGGTCCAGCCTGG
GAGGTCCCTGAGACTCTCCTGTGCAGCCTCTGGATTCACCTTCAGTAGCTATACTATGCACTGGGTCCGC
CAGGCTCCAGGCAAGGGGCTGGAGTGGGTGACATTTATATCATATGATGGAAACAATAAATACTACGCAG
ACTCCGTGAAGGGCCGATTCACCATCTCCAGAGACAATTCCAAGAACACGCTGTATCTGCAAATGAACAG
CCTGAGAGCTGAGGACACGGCTATATATTACTGTGCGAGGACCGGCTGGCTGGGGCCCTTTGACTACTGG
GGCCAGGGAACCCTGGTCACCGTCTCGAGCGGTGGAGGCGGATCTGGCGGAGGTGGTTCCGGCGGTGGCG
GCTCCGGTGGAGGCGGTTCTGAAATTGTGTTGACGCAGTCTCCAGGCACCCTGTCTTTGTCTCCAGGGGA
AAGAGCCACCCTCTCCTGCAGGGCCAGTCAGAGTGTTGGCAGCAGCTACTTAGCCTGGTACCAGCAGAAA
CCTGGCCAGGCTCCCAGGCTCCTCATCTATGGTGCATTCAGCAGGGCCACTGGCATCCCAGACAGGTTCA
GTGGCAGTGGGTCTGGGACAGACTTCACTCTCACCATCAGCAGACTGGAGCCTGAAGATTTTGCAGTGTA
TTACTGTCAGCAGTATGGTAGCTCACCGTGGACGTTCGGCCAAGGGACCAAGGTGGAAATCAAA
SEQ ID 54: PD215A1 x CT4 aa
QEQLLESGGGLVQPGGSLRLSCTASGFSFSSYWMCWVRQAPGKGLEWIGCITTGSGSTYYASWAKRRFTI
SKDNSKNTVTLQMNSLRAEDTAVYYCTRAFDLWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKR
VEPKSCDKTHTCPPCPAPEAAGAPSVFLFPPKPKDTLMISRTPEVTCVVVDVSHEDPEVKFNWYVDGVEV
HNAKTKPREEQYNSTYRVVSVLTVLHQDWLNGKEYKCAVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQGNVFS
CSVMHEALHNHYTQKSLSLSPGGGGGSGGGGSQVQLVESGGGVVQPGRSLRLSCAASGFTFSSYTMHWVR
QAPGKGLEWVTFISYDGNNKYYADSVKGRFTISRDNSKNTLYLQMNSLRAEDTAIYYCARTGWLGPFDYW
GQGTLVTVSSGGGGSGGGGSGGGGSGGGGSEIVLTQSPGTLSLSPGERATLSCRASQSVGSSYLAWYQQK
PGQAPRLLIYGAFSRATGIPDRFSGSGSGTDFTLTISRLEPEDFAVYYCQQYGSSPWTFGQGTKVEIK
SEQ ID 55: PD215A1 Light Chain nt
GACATCCAGATGACCCAGTCTCCATCCTCCCTGTCTGCATCTGTAGGAGACAGAGTCACCATCACTTGCC
AGGCCAGTCAGAGCATTTACAGCTACTTAAACTGGTATCAGCAGAAACCAGGGAAAGCCCCTAAGCTCCT
GATCTATGGTGCATCCAATCTGGCATCTGGGGTCCCATCAAGGTTCAGTGGCAGTGGATCTGGGACAGAT
TTCACTCTCACCATCAGCAGTCTGCAACCTGAAGATTTTGCAACTTACTACTGTCAAAGCAGTTGGTTGA
GTGGTGCTGTTGGTAATGCTTTCGGCGGAGGGACCAAGGTGGAGATCAAACGTACGGTGGCTGCACCATC
TGTCTTCATCTTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGTAACTCCCAGG
AGAGTGTCACAGAGCAGGACAGCAAGGACAGCACCTACAGCCTCAGCAGCACCCTGACGCTGAGCAAAGC
AGACTACGAGAAACACAAAGTCTACGCCTGCGAAGTCACCCATCAGGGCCTGAGCTCGCCCGTCACAAAG
AGCTTCAACAGGGGAGAGTGT
SEQ IF 56: PD215A1 Light Chain aa
DIQMTQSPSSLSASVGDRVTITCQASQSIYSYLNWYQQKPGKAPKLLIYGASNLASGVPSRFSGSGSGTD
FTLTISSLQPEDFATYYCQSSWLSGAVGNAFGGGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTK
SFNRGEC
SEQ ID 57: PD220F6 x CT4 nt
CAGGAGCAGGTGAAGGAGACCGGGGGAGGCTTGGTACAGCCTGGGGGGTCCCTGAGACTCTCCTGTGCAG
CCTCTGGATTCACCATCAGCAGCTATGGAGTGAGCTGGGTCCGCCAGGCTCCAGGGAAGGGGCTGGAGTG
GGTCGCATTGATTTTTCCCGGGATTGGTTTCAAAGACTACGCGAGCTGGGTGAATGGCCGGTTCACCCTC
TCCAGCGACAACGCCCAGAACACTGTGGAACTGCAAATGAACAGCCTGAGAGCCGAGGACACGGCCGTAT
ATTACTGTGCGAGAGATTTGGACTTGTGGGGCCAGGGAACCCTGGTCACCGTCTCGAGCGCTAGCACCAA
GGGCCCATCGGTCTTCCCCCTGGCACCCTCCTCCAAGAGCACCTCTGGGGGCACAGCGGCCCTGGGCTGC
CTGGTCAAGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCCCTCCAG
CAGCTTGGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAAGCCGCGGGGGCAC
CGTCAGTCTTCCTCTTCCCCCCAAAACCCAAGGACACCCTCATGATCTCCCGGACCCCTGAGGTCACATG
CGTGGTGGTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTCCTCACCG
TCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGACTCCGA
CGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCG
GTGGAGGGTCCGGCGGTGGTGGATCCCAGGTGCAATTGGTGGAGTCTGGGGGAGGCGTGGTCCAGCCTGG
GAGGTCCCTGAGACTCTCCTGTGCAGCCTCTGGATTCACCTTCAGTAGCTATACTATGCACTGGGTCCGC
CAGGCTCCAGGCAAGGGGCTGGAGTGGGTGACATTTATATCATATGATGGAAACAATAAATACTACGCAG
ACTCCGTGAAGGGCCGATTCACCATCTCCAGAGACAATTCCAAGAACACGCTGTATCTGCAAATGAACAG
CCTGAGAGCTGAGGACACGGCTATATATTACTGTGCGAGGACCGGCTGGCTGGGGCCCTTTGACTACTGG
GGCCAGGGAACCCTGGTCACCGTCTCGAGCGGTGGAGGCGGATCTGGCGGAGGTGGTTCCGGCGGTGGCG
GCTCCGGTGGAGGCGGTTCTGAAATTGTGTTGACGCAGTCTCCAGGCACCCTGTCTTTGTCTCCAGGGGA
AAGAGCCACCCTCTCCTGCAGGGCCAGTCAGAGTGTTGGCAGCAGCTACTTAGCCTGGTACCAGCAGAAA
CCTGGCCAGGCTCCCAGGCTCCTCATCTATGGTGCATTCAGCAGGGCCACTGGCATCCCAGACAGGTTCA
GTGGCAGTGGGTCTGGGACAGACTTCACTCTCACCATCAGCAGACTGGAGCCTGAAGATTTTGCAGTGTA
TTACTGTCAGCAGTATGGTAGCTCACCGTGGACGTTCGGCCAAGGGACCAAGGTGGAAATCAAA
SEQ ID 58: PD220F6 x CT4 aa
QEQVKETGGGLVQPGGSLRLSCAASGFTISSYGVSWVRQAPGKGLEWVALIFPGIGFKDYASWVNGRFTL
SSDNAQNTVELQMNSLRAEDTAVYYCARDLDLWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKR
VEPKSCDKTHTCPPCPAPEAAGAPSVFLFPPKPKDTLMISRTPEVTCVVVDVSHEDPEVKFNWYVDGVEV
HNAKTKPREEQYNSTYRVVSVLTVLHQDWLNGKEYKCAVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQGNVFS
CSVMHEALHNHYTQKSLSLSPGGGGGSGGGGSQVQLVESGGGVVQPGRSLRLSCAASGFTFSSYTMHWVR
QAPGKGLEWVTFISYDGNNKYYADSVKGRFTISRDNSKNTLYLQMNSLRAEDTAIYYCARTGWLGPFDYW
GQGTLVTVSSGGGGSGGGGSGGGGSGGGGSEIVLTQSPGTLSLSPGERATLSCRASQSVGSSYLAWYQQK
PGQAPRLLIYGAFSRATGIPDRFSGSGSGTDFTLTISRLEPEDFAVYYCQQYGSSPWTFGQGTKVEIK
SEQ ID 59: PD220F6 Light Chain nt
GACATCCAGATGACCCAGTCTCCATCCTCCCTGTCTGCATCTGTAGGAGACAGAGTCACCATCACTTGCC
AGTCCAGTCCGAGTGTTTATAGTAACTACTTATCCTGGTATCAGCAGAAACCAGGGAAAGTTCCTAAGCT
CCTGATCTATTATGCATCCACTCTGGCATCTGGGGTCCCATCTCGGTTCAGTGGCAGTGGATCTGGGACA
GATTTCACTCTCACCATCAGCAGCCTGCAGCCTGAAGATGTTGCAACTTATTACTGTGCAGGCGGTTATA
GTAGTAGTACTCGTGCTTTCGGCGGAGGGACCAAGGTGGAGATCAAACGTACGGTGGCTGCACCATCTGT
CTTCATCTTCCCGCCATCTGATGAGCAGTTGAAATCTGGAACTGCCTCTGTTGTGTGCCTGCTGAATAAC
TTCTATCCCAGAGAGGCCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGACGCTGAGCAAAGCAGA
CTACGAGAAACACAAAGTCTACGCCTGCGAAGTCACCCATCAGGGCCTGAGCTCGCCCGTCACAAAGAGC
TTCAACAGGGGAGAGTGT
SEQ ID 60: PD220F6 Light Chain aa
DIQMTQSPSSLSASVGDRVTITCQSSPSVYSNYLSWYQQKPGKVPKLLIYYASTLASGVPSRFSGSGSGT
DFTLTISSLQPEDVATYYCAGGYSSSTRAFGGGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTKS
FNRGEC
SEQ ID 61: PD225G11 x CT4 nt
GAGGTGCAGCTGTTGGAGTCTGGGGGAGGCTTGGTACAGCCTGGGGGGTCCCTGAGACTCTCCTGTGCAG
CCTCTGGATTCACCTTTAGCAGCAGCCACTGGATATGCTGGGTCCGCCAGGCTCCAGGGAAGGGGCTGGA
GTGGATCGCATGCATTTATACTGGTAGTATTGATGTCTTTTACTACGCGAGCTGGGCGAAAGGCCGGTTC
ACCATCTCCAGAGACAATTCCAAGAACACGCTGTATCTGCAAATGAACAGCCTGAGAGCCGAGGACACGG
CCGTATATTACTGTGCGAGAGCCGCTAATACTGATACTACCTACTTTAACTTGTGGGGCCAGGGAACCCT
GGTCACCGTCTCGAGCGCTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAACTCACACATGCCCACCGT
GCCCAGCACCTGAAGCCGCGGGGGCACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGCAAGGAGTACAAGTGCGC
GGTCTCCAACAAAGCCCTCCCAGCCCCCATCGAGAAAACCATCTCCAAAGCCAAAGGGCAGCCCCGAGAA
CCACAGGTGTACACCCTGCCCCCATCCCGGGATGAGCTGACCAAGAACCAGGTCAGCCTGACCTGCCTGG
TCAAAGGCTTCTATCCCAGCGACATCGCCGTGGAGTGGGAGAGCAATGGGCAGCCGGAGAACAACTACAA
GACCACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACGCAGA
AGAGCCTCTCCCTGTCTCCGGGTGGCGGTGGAGGGTCCGGCGGTGGTGGATCCCAGGTGCAATTGGTGGA
GTCTGGGGGAGGCGTGGTCCAGCCTGGGAGGTCCCTGAGACTCTCCTGTGCAGCCTCTGGATTCACCTTC
AGTAGCTATACTATGCACTGGGTCCGCCAGGCTCCAGGCAAGGGGCTGGAGTGGGTGACATTTATATCAT
ATGATGGAAACAATAAATACTACGCAGACTCCGTGAAGGGCCGATTCACCATCTCCAGAGACAATTCCAA
GAACACGCTGTATCTGCAAATGAACAGCCTGAGAGCTGAGGACACGGCTATATATTACTGTGCGAGGACC
GGCTGGCTGGGGCCCTTTGACTACTGGGGCCAGGGAACCCTGGTCACCGTCTCGAGCGGTGGAGGCGGAT
CTGGCGGAGGTGGTTCCGGCGGTGGCGGCTCCGGTGGAGGCGGTTCTGAAATTGTGTTGACGCAGTCTCC
AGGCACCCTGTCTTTGTCTCCAGGGGAAAGAGCCACCCTCTCCTGCAGGGCCAGTCAGAGTGTTGGCAGC
AGCTACTTAGCCTGGTACCAGCAGAAACCTGGCCAGGCTCCCAGGCTCCTCATCTATGGTGCATTCAGCA
GGGCCACTGGCATCCCAGACAGGTTCAGTGGCAGTGGGTCTGGGACAGACTTCACTCTCACCATCAGCAG
ACTGGAGCCTGAAGATTTTGCAGTGTATTACTGTCAGCAGTATGGTAGCTCACCGTGGACGTTCGGCCAA
GGGACCAAGGTGGAAATCAAA
SEQ ID 62: PD225G11 x CT4 aa
EVQLLESGGGLVQPGGSLRLSCAASGFTFSSSHWICWVRQAPGKGLEWIACIYTGSIDVFYYASWAKGRF
TISRDNSKNTLYLQMNSLRAEDTAVYYCARAANTDTTYFNLWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHK
PSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPRE
PQVYTLPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGGGGGSGGGGSQVQLVESGGGVVQPGRSLRLSCAASGFTF
SSYTMHWVRQAPGKGLEWVTFISYDGNNKYYADSVKGRFTISRDNSKNTLYLQMNSLRAEDTAIYYCART
GWLGPFDYWGQGTLVTVSSGGGGSGGGGSGGGGSGGGGSEIVLTQSPGTLSLSPGERATLSCRASQSVGS
SYLAWYQQKPGQAPRLLIYGAFSRATGIPDRFSGSGSGTDFTLTISRLEPEDFAVYYCQQYGSSPWTFGQ
GTKVEIK
SEQ ID 63: PD225G11 Light Chain nt
GCCTATGATATGACCCAGTCTCCATCCTCCCTGTCTGCATCTGTAGGAGACAGAGTCACCATCAATTGCC
AGGCCAGTCAGAGCATTAACAACCAACTATCCTGGTATCAGCAGAAACCAGGGAAAGTTCCTAAGCTCCT
GATCTATGGTGCATCCACTCTGGCATCTGGGGTCCCATCTCGGTTCACCGGCAGTGGATCTGGGACAGAT
TTCACTCTCACCATCAGCAGCCTGCAGCCTGAAGATGTTGCAACTTATTACTGTCATGTTCATTATTGCA
GTGGTGGTAGTTGTTTTTGGGCTTTCGGCGGAGGGACCAAGGTGGAGATCAAACGTACGGTGGCTGCACC
ATCTGTCTTCATCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAGCACCCTGACGCTGAGCAA
AGCAGACTACGAGAAACACAAAGTCTACGCCTGCGAAGTCACCCATCAGGGCCTGAGCTCGCCCGTCACA
AAGAGCTTCAACAGGGGAGAGTGT
SEQ ID 64: PD225G11 Light Chain aa
AYDMTQSPSSLSASVGDRVTINCQASQSINNQLSWYQQKPGKVPKLLIYGASTLASGVPSRFTGSGSGTD
FTLTISSLQPEDVATYYCHVHYCSGGSCFWAFGGGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSPVT
KSFNRGEC
SEQ ID 65: CT4 heavy chain signal peptide nucleotide sequence nt
ATGAAACACCTGTGGTTCTTCCTCCTCCTGGTGGCAGCTCCCAGATGGGTCCTGTCC
SEQ ID 66: CT4 heavy chain signal peptide nucleotide sequence aa
MKHLWFFLLLVAAPRWVLS
SEQ ID 67: PL230/PD224 Heavy chain signal peptide nt
ATGGCTGTCTTGGGGCTGCTCTTCTGCCTGGTGACATTCCCAAGCTGTGTCCTATCC
SEQ ID 68: PL230/PD224 Heavy chain signal peptide aa
MAVLGLLFCLVTFPSCVLS
SEQ ID 69: PL230/PD224 Light chain signal peptide nt
ATGAGGGCCCCTGCTCAGTTTCTTGGCTTCTTGCTTTTCTGGATTCCAGCCTCCAGAAGT
SEQ ID 70: PL230/PD224 Light chain signal peptide aa
MRAPAQFLGFLLFWIPASRS
SEQ ID 71: CT4 scFv nt
CAGGTGCAATTGGTGGAGTCTGGGGGAGGCGTGGTCCAGCCTGGGAGGTCCCTGAGACTCTCCTGTGCAG
CCTCTGGATTCACCTTCAGTAGCTATACTATGCACTGGGTCCGCCAGGCTCCAGGCAAGGGGCTGGAGTG
GGTGACATTTATATCATATGATGGAAACAATAAATACTACGCAGACTCCGTGAAGGGCCGATTCACCATC
TCCAGAGACAATTCCAAGAACACGCTGTATCTGCAAATGAACAGCCTGAGAGCTGAGGACACGGCTATAT
ATTACTGTGCGAGGACCGGCTGGCTGGGGCCCTTTGACTACTGGGGCCAGGGAACCCTGGTCACCGTCTC
GAGCGGTGGAGGCGGATCTGGCGGAGGTGGTTCCGGCGGTGGCGGCTCCGGTGGAGGCGGTTCTGAAATT
GTGTTGACGCAGTCTCCAGGCACCCTGTCTTTGTCTCCAGGGGAAAGAGCCACCCTCTCCTGCAGGGCCA
GTCAGAGTGTTGGCAGCAGCTACTTAGCCTGGTACCAGCAGAAACCTGGCCAGGCTCCCAGGCTCCTCAT
CTATGGTGCATTCAGCAGGGCCACTGGCATCCCAGACAGGTTCAGTGGCAGTGGGTCTGGGACAGACTTC
ACTCTCACCATCAGCAGACTGGAGCCTGAAGATTTTGCAGTGTATTACTGTCAGCAGTATGGTAGCTCAC
CGTGGACGTTCGGCCAAGGGACCAAGGTGGAAATCAAA
SEQ ID 72: CT4 scFv aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSGGGGSGGGGSGGGGSGGGGSEI
VLTQSPGTLSLSPGERATLSCRASQSVGSSYLAWYQQKPGQAPRLLIYGAFSRATGIPDRFSGSGSGTDF
TLTISRLEPEDFAVYYCQQYGSSPWTFGQGTKVEIK
SEQ ID73: PD224D1 scFv nt
GAGGTGCAGCTGGTGGAGTCTGGGGGAGGCTTGGTCCAGCCTGGGGGGTCCCTGAGACTCTCCTGTACAGC
CTCTGGATTCTCCCTCAGTAGCTATGCAATGAGCTGGGTCCGCCAGGCTCCAGGGAAGGGGCTGGAGTACATCGGCTACA
TTGGTGATACTACTGGCATAGCCTACGCGAGCTGGGCGAATGGCAGATTCACCATCTCCAAAGACAATACCAAGAACACG
GTGGATCTTCAAATGAACAGCCTGAGAGCCGAGGACACGGCTGTGTATTACTGTGCGAGAGGCTGGTCCTACTTAGACAT
CTGGGGCCAAGGGACCCTGGTCACCGTCTCGAGCGGTGGAGGCGGATCTGGCGGAGGTGGTTCCGGCGGTGGCGGCTCCG
GTGGAGGCGGCTCTGCCCTTGTGATGACCCAGTCTCCATCCTCCCTGTCTGCATCTGTAGGAGACAGAGTCACCATCACT
TGCCAGGCCAGTCAGAACATTTACAGCAATTTAGCCTGGTATCAGCAGAAACCAGGGAAAGTTCCTAAGCTCCTGATCTA
TCAGGCCTCCACTCTGGCATCTGGGGTCCCATCTCGGTTCAGTGGCAGTGGATATGGGACAGATTTCACTCTCACCATCA
GCAGCCTGCAGCCTGAAGATGTTGCAACTTATTACTGTCAAGGCGGTTATTATAGTGCTGCCCTTAATACTTTCGGCGGA
GGGACCAAGGTGGAGATCAAA
SEQ ID 74: PD224D1 scFv aa
EVQLVESGGGLVQPGGSLRLSCTASGFSLSSYAMSWVRQAPGKGLEYIGYIGDTTGIAYASWANGRFTISKDNTKNTVDLQMNS
LRAEDTAVYYCARGWSYLDIWGQGTLVTVSSGGGGSGGGGSGGGGSGGGGSALVMTQSPSSLSASVGDRVTITCQASQNIYSN
LAWYQQKPGKVPKLLIYQASTLASGVPSRFSGSGYGTDFTLTISSLQPEDVATYYCQGGYYSAALNTFGGGTKVEIK
SEQ ID 75: PD206F12 scFv nt
GAGGTGCAGCTGGTGGAGTCTGGGGGAGGCTTGGTCCAGCCTGGGGGGTCCCTGAGACTCTCCTGTGCAG
CCTCTGGATTCGACTTCAGTAGCGGCTACTGGATATGCTGGGTCCGCCAGGCTCCAGGGAAGGGGCTGGA
GTTGATCGCATGCATTTATGCTGGTACTAGTGGTAGTACTTCCTACGCGAGCTGGGCGAGAGGCAGATTC
ACCATCTCCGAAACCTCCAAGAACACGGTGACTCTTCAAATGAACAGCCTGAGAGCCGAGGACTCGGCTG
TGTATTACTGTGCGAGAAATCTTTACACTTACAATAGCTTGTGGGGCCAGGGAACCCTGGTCACCGTCTC
GAGCGGTGGAGGCGGATCTGGCGGAGGTGGTTCCGGCGGTGGCGGCTCCGGTGGAGGCGGCTCTGACATC
CAGATGACCCAGTCTCCTTCCACCCTGTCTGCATCTGTAGGAGACAGAGTCACCATCACTTGCCAGTCCA
GTCAGAGTGTTTATGATAACAACTGGTTAGCCTGGTATCAGCAGAAACCAGGGAAAGCCCCTAAGCTCCT
GATCTATACAGTATCCACTCTGGCATCTGGGGTCCCATCAAGGTTCAGCGGCAGTGGATCTGGGACAGAA
TTCACTCTCACCATCAGCAGCCTGCAGCCTGATGATTTTGCAACTTATTACTGCCAAGGCACTTATTATA
GTAGTGGTTGGAACTTTGCTTTCGGCGGAGGGACCAAGGTGGAGATCAAA
SEQ ID 76: PD206F12 scFv aa
EVQLVESGGGLVQPGGSLRLSCAASGFDFSSGYWICWVRQAPGKGLELIACIYAGTSGSTSYASWARGRF
TISETSKNTVTLQMNSLRAEDSAVYYCARNLYTYNSLWGQGTLVTVSSGGGGSGGGGSGGGGSGGGGSDI
QMTQSPSTLSASVGDRVTITCQSSQSVYDNNWLAWYQQKPGKAPKLLIYTVSTLASGVPSRFSGSGSGTE
FTLTISSLQPDDFATYYCQGTYYSSGWNFAFGGGTKVEIK
SEQ ID 77: PD215A1 scFv nt
CAGGAGCAGCTGTTGGAGTCTGGGGGAGGCTTGGTACAGCCTGGGGGGTCCCTGAGACTCTCCTGTACAG
CCTCTGGATTCTCCTTTAGCAGCTACTGGATGTGCTGGGTCCGCCAGGCTCCAGGGAAGGGGCTGGAGTG
GATCGGATGCATTACGACTGGTAGTGGTAGCACTTACTACGCGAGCTGGGCGAAGCGCCGGTTCACCATC
TCCAAAGACAATTCCAAGAACACGGTGACTCTGCAAATGAACAGCCTGAGAGCCGAGGACACGGCCGTAT
ATTACTGTACGAGAGCATTTGACTTGTGGGGCCAGGGAACCCTGGTCACCGTCTCGAGCGGTGGAGGCGG
ATCTGGCGGAGGTGGTTCCGGCGGTGGCGGCTCCGGTGGAGGCGGCTCTGACATCCAGATGACCCAGTCT
CCATCCTCCCTGTCTGCATCTGTAGGAGACAGAGTCACCATCACTTGCCAGGCCAGTCAGAGCATTTACA
GCTACTTAAACTGGTATCAGCAGAAACCAGGGAAAGCCCCTAAGCTCCTGATCTATGGTGCATCCAATCT
GGCATCTGGGGTCCCATCAAGGTTCAGTGGCAGTGGATCTGGGACAGATTTCACTCTCACCATCAGCAGT
CTGCAACCTGAAGATTTTGCAACTTACTACTGTCAAAGCAGTTGGTTGAGTGGTGCTGTTGGTAATGCTT
TCGGCGGAGGGACCAAGGTGGAGATCAAA
SEQ ID 78: PD215A1 scFv aa
QEQLLESGGGLVQPGGSLRLSCTASGFSFSSYWMCWVRQAPGKGLEWIGCITTGSGSTYYASWAKRRFTI
SKDNSKNTVTLQMNSLRAEDTAVYYCTRAFDLWGQGTLVTVSSGGGGSGGGGSGGGGSGGGGSDIQMIQS
PSSLSASVGDRVTITCQASQSIYSYLNWYQQKPGKAPKLLIYGASNLASGVPSRFSGSGSGTDFTLTISS
LQPEDFATYYCQSSWLSGAVGNAFGGGTKVEIK
SEQ ID 79: PD225G11 scFv nt
GAGGTGCAGCTGTTGGAGTCTGGGGGAGGCTTGGTACAGCCTGGGGGGTCCCTGAGACTCTCCTGTGCAG
CCTCTGGATTCACCTTTAGCAGCAGCCACTGGATATGCTGGGTCCGCCAGGCTCCAGGGAAGGGGCTGGA
GTGGATCGCATGCATTTATACTGGTAGTATTGATGTCTTTTACTACGCGAGCTGGGCGAAAGGCCGGTTC
ACCATCTCCAGAGACAATTCCAAGAACACGCTGTATCTGCAAATGAACAGCCTGAGAGCCGAGGACACGG
CCGTATATTACTGTGCGAGAGCCGCTAATACTGATACTACCTACTTTAACTTGTGGGGCCAGGGAACCCT
GGTCACCGTCTCGAGCGGTGGAGGCGGATCTGGCGGAGGTGGTTCCGGCGGTGGCGGCTCCGGTGGAGGC
GGCTCTGCCTATGATATGACCCAGTCTCCATCCTCCCTGTCTGCATCTGTAGGAGACAGAGTCACCATCA
ATTGCCAGGCCAGTCAGAGCATTAACAACCAACTATCCTGGTATCAGCAGAAACCAGGGAAAGTTCCTAA
GCTCCTGATCTATGGTGCATCCACTCTGGCATCTGGGGTCCCATCTCGGTTCACCGGCAGTGGATCTGGG
ACAGATTTCACTCTCACCATCAGCAGCCTGCAGCCTGAAGATGTTGCAACTTATTACTGTCATGTTCATT
ATTGCAGTGGTGGTAGTTGTTTTTGGGCTTTCGGCGGAGGGACCAAGGTGGAGATCAAA
SEQ ID 80: PD225G11 scFv aa
EVQLLESGGGLVQPGGSLRLSCAASGFTFSSSHWICWVRQAPGKGLEWIACIYTGSIDVFYYASWAKGRF
TISRDNSKNTLYLQMNSLRAEDTAVYYCARAANTDTTYFNLWGQGTLVTVSSGGGGSGGGGSGGGGSGGG
GSAYDMTQSPSSLSASVGDRVTINCQASQSINNQLSWYQQKPGKVPKLLIYGASTLASGVPSRFTGSGSG
TDFTLTISSLQPEDVATYYCHVHYCSGGSCFWAFGGGTKVEIK
SEQ ID 81: PL23006 scFv nt
CAGTCGGTGGAGGAGTCTGGGGGAGGCTTGGTCCAGCCTGGGGGGTCCCTGAGACTCTCCTGTACCGCCT
CTGGAATCGACCTTAATACCTACGACATGATCTGGGTCCGCCAGGCTCCAGGCAAGGGGCTAGAGTGGGT
TGGAATCATTACTTATAGTGGTAGTAGATACTACGCGAACTGGGCGAAAGGCCGATTCACCATCTCCAAA
GACAATACCAAGAACACGGTGTATCTGCAAATGAACAGCCTGAGAGCTGAGGACACGGCTGTGTATTACT
GTGCGAGAGATTATATGAGTGGTTCCCACTTGTGGGGCCAGGGAACCCTGGTCACCGTCTCTTCCGGTGG
AGGCGGTTCAGGCGGAGGTGGAAGTGGTGGTGGCGGCTCTGGAGGCGGCGGATCTGCCTATGATATGACC
CAGTCTCCATCTTCCGTGTCTGCATCTGTAGGAGACAGAGTCACCATCAAGTGTCAGGCCAGTGAGGACA
TTTATAGCTTCTTGGCCTGGTATCAGCAGAAACCAGGGAAAGCCCCTAAGCTCCTGATCCATTCTGCATC
CTCTCTGGCATCTGGGGTCCCATCAAGGTTCAGCGGCAGTGGATCTGGGACAGATTTCACTCTCACCATC
AGCAGCCTGCAGCCTGAAGATTTTGCAACTTACTATTGTCAACAGGGTTATGGTAAAAATAATGTTGATA
ATGCTTTCGGCGGAGGGACCAAGGTGGAGATCAAA
SEQ ID 82: PL23006 scFv aa
QSVEESGGGLVQPGGSLRLSCTASGIDLNTYDMIWVRQAPGKGLEWVGIITYSGSRYYANWAKGRFTISK
DNTKNTVYLQMNSLRAEDTAVYYCARDYMSGSHLWGQGTLVTVSSGGGGSGGGGSGGGGSGGGGSAYDMT
QSPSSVSASVGDRVTIKCQASEDIYSFLAWYQQKPGKAPKLLIHSASSLASGVPSRFSGSGSGTDFTLTI
SSLQPEDFATYYCQQGYGKNNVDNAFGGGTKVEIK
SEQ ID 83: PL231H2 scFv nt
CAGGTGCAGCTGGTGGAGTCTGGGGGAGGCCTGGTCCAGCCTGGGGGCTCCCTGAGACTCTCCTGTACAA
CTTCTGGAATCGACCTTAGTACCTACGACATGATCTGGGTCCGCCAGGCTCCAGGCAAGGGGCTAGAGTG
GGTGGGAATCATTAGTTATGTTGGTAACACATACTACGCGAGCTGGGCGAAAGGCCGATTCACCCTCTCC
AAAGACAATACCTCGACCACGGTGGATCTGCAAATGAACAGCCTGAGAGCTGAGGACACGGCTGTGTATT
ACTGTGCGAGAGATTTTATTAGTGGTTCCCACTTGTGGGGCCAGGGAACCCTGGTCACCGTCTCGAGCGG
TGGAGGCGGATCTGGCGGAGGTGGTTCCGGCGGTGGCGGCTCCGGTGGAGGCGGTTCTGCCTATGATATG
ACCCAGTCTCCATCTTCCGTGTCTGCATCTGTAGGAGACAGAGTCACCATCAATTGTCAGGCCAGTGAGA
GCATTAGTAGCTTCTTATCCTGGTATCAGCAGAAACCAGGGAAAGCCCCTAAGCTCCTGATCTTTTCTGC
ATCCACTCTGGCATCTGGGGTCCCATCAAGGTTCAGCGGCAGTGGATCTGGGACAGATTTCACTCTCACC
ATCAGCAGCCTGCAGCCTGAAGATTTTGCAACTTACTATTGTCAACAGGGTTATAGTAAAAGTAATGTGG
ATAATGCTTTCGGCGGAGGGACCAAGGTGGAGATCAAA
SEQ ID 84: PL231H2 scFv aa
QVQLVESGGGLVQPGGSLRLSCTTSGIDLSTYDMIWVRQAPGKGLEWVGIISYVGNTYYASWAKGRFTLS
KDNTSTTVDLQMNSLRAEDTAVYYCARDFISGSHLWGQGTLVTVSSGGGGSGGGGSGGGGSGGGGSAYDM
TQSPSSVSASVGDRVTINCQASESISSFLSWYQQKPGKAPKLLIFSASTLASGVPSRFSGSGSGTDFTLT
ISSLQPEDFATYYCQQGYSKSNVDNAFGGGTKVEIK
SEQ ID 85: PL221G5 scFv nt
GAGGTGCAGCTGTTGGAGTCTGGGGGAGGCTTGGTACAGCCTGGGGGGTCCCTGAGACTCTCCTGTGCAG
CCTCTGGATTCTCCTTCAGTAGCGGGTACGACATGTGCTGGGTCCGCCAGGCTCCAGGGAAGGGGCTGGA
GTGGATCGCATGCATTGCTGCTGGTAGTGCTGGTATCACTTACGACGCGAACTGGGCGAAAGGCCGGTTC
ACCATCTCCAGAGACAATTCCAAGAACACGCTGTATCTGCAAATGAACAGCCTGAGAGCCGAGGACACGG
CCGTATATTACTGTGCGAGATCGGCGTTTTCGTTCGACTACGCCATGGACCTCTGGGGCCAGGGAACCCT
GGTCACCGTCTCGAGCGGTGGAGGCGGATCTGGCGGAGGTGGTTCCGGCGGTGGCGGCTCCGGTGGAGGC
GGCTCTGACATCCAGATGACCCAGTCTCCTTCCACCCTGTCTGCATCTGTAGGAGACAGAGTCACCATCA
CTTGCCAGGCCAGTCAGAGCATTAGTTCCCACTTAAACTGGTATCAGCAGAAACCAGGGAAAGCCCCTAA
GCTCCTGATCTATAAGGCATCCACTCTGGCATCTGGGGTCCCATCAAGGTTCAGCGGCAGTGGATCTGGG
ACAGAATTTACTCTCACCATCAGCAGCCTGCAGCCTGATGATTTTGCAACTTATTACTGCCAACAGGGTT
ATAGTTGGGGTAATGTTGATAATGTTTTCGGCGGAGGGACCAAGGTGGAGATCAAA
SEQ ID 86: PL221G5 scFv aa
EVQLLESGGGLVQPGGSLRLSCAASGFSFSSGYDMCWVRQAPGKGLEWIACIAAGSAGITYDANWAKGRF
TISRDNSKNTLYLQMNSLRAEDTAVYYCARSAFSFDYAMDLWGQGTLVTVSSGGGGSGGGGSGGGGSGGG
GSDIQMTQSPSTLSASVGDRVTITCQASQSISSHLNWYQQKPGKAPKLLIYKASTLASGVPSRFSGSGSG
TEFTLTISSLQPDDFATYYCQQGYSWGNVDNVFGGGTKVEIK
SEQ ID 87: PL004B5 scFv nt
GAGGTGCAGCTGGTGGAGTCTGGGGGAGGCTTGGTCCAGCCTGGGGGGTCCCTGAGACTCTCCTGTGCAG
CCTCTGGATTCTCCCTCAGTAGCTACTGGATGAGCTGGGTCCGCCAGGCTCCAGGGAAGGGGCTGGAGTG
GATCGGAGTCATTGATACTAATGTTTATATATACTACGCGAACTGGGCAAAAGGCAGATTCACCATCTCC
AGAGACAATTCCAAGAACACGCTGTATCTTCAAATGAACAGCCTGAGAGCCGAGGACACGGCTGTGTATT
ACTGTGCGAGATATGTGGGTAATAATGATGATTATATTAACTTGTGGGGCCAGGGAACCCTGGTCACCGT
CTCGAGCGGTGGAGGCGGATCTGGCGGAGGTGGTTCCGGCGGTGGCGGCTCCGGTGGAGGCGGCTCTGAC
ATCCAGATGACCCAGTCTCCTTCCACCCTGTCTGCATCTGTAGGAGACAGAGTCACCATCACTTGCCAGT
CCAGTCAGAGTGTTTATAATGGCTACTGGTTATCCTGGTATCAGCAGAAACCAGGGAAAGCCCCTAAGCT
CCTGATCTATGGTGCATCCACTCTGGCATCTGGGGTCCCATCAAGGTTCAGCGGCAGTGGATCTGGGACA
GAATTCACTCTCACCATCAGCAGCCTGCAGCCTGATGATTTTGCAACTTATTACTGCCTAGGCAGTTATA
CTAGTAGTACTGAGAACTCTTTCGGCGGAGGGACCAAGGTGGAGATCAAA
SEQ ID 88: PL004B5 scFv aa
EVQLVESGGGLVQPGGSLRLSCAASGFSLSSYWMSWVRQAPGKGLEWIGVIDTNVYIYYANWAKGRFTIS
RDNSKNTLYLQMNSLRAEDTAVYYCARYVGNNDDYINLWGQGTLVTVSSGGGGSGGGGSGGGGSGGGGSD
IQMTQSPSTLSASVGDRVTITCQSSQSVYNGYWLSWYQQKPGKAPKLLIYGASTLASGVPSRFSGSGSGT
EFTLTISSLQPDDFATYYCLGSYTSSTENSFGGGTKVEIK
SEQ ID 89: PL004B9 scFv nt
GAGGTGCAGCTGGTGGAGTCTGGGGGAGGCTTGGTCCAGCCTGGGGGGTCCCTGAGACTCTCCTGTACAG
TGTCTGGAATCGACCTCAGTGTCATCAATATGGGCTGGGTCCGCCAGGCTCCAGGGAAGGGGCTGGAGTG
GATCGGAACCATTACTTATGTTGGTAACACATATTACGCGAGCTGGGCGAAAGGCAGATTCACCATCTCC
AAAACCTCGACCACGGTGGATCTTAAAATCACCAGTCCGACAACCGAGGACACGGCTGTGTATTACTGTG
CGAGAGAATCTGGTACTATTTATTACAGTTACTTTAACTTGTGGGGCCAAGGGACCCTGGTCACCGTCTC
GAGCGGTGGAGGCGGATCTGGCGGAGGTGGTTCCGGCGGTGGCGGCTCCGGTGGAGGCGGCTCTGCATTC
GAATTGACCCAGTCTCCATCCTCCCTGTCTGCATCTGTAGGAGACAGAGTCACCATCAAGTGCCAGGCCA
GTGAAAGCATTAGCAACTACTTATCCTGGTATCAGCAGATTCCAGGGAAAGTTCCTAAGCTCCTGATCTA
TTATGCATCCAATCTGGCATCTGGGGTCCCATCTCGGTTCAGTGGCAGTGGATCTGGGACAGATTTCACT
CTCACCATCAGCAGCCTGCAGCCTGAAGATGTTGCAACTTATTACTGTCAAAGCTATTATGGTGGTGGTA
GTGCCTATACTTTCGGCGGAGGGACCAAGGTGGAGATCAAA
SEQ ID 90: PL004B9 scFv aa
EVQLVESGGGLVQPGGSLRLSCTVSGIDLSVINMGWVRQAPGKGLEWIGTITYVGNTYYASWAKGRFTIS
KTSTTVDLKITSPTTEDTAVYYCARESGTIYYSYFNLWGQGTLVTVSSGGGGSGGGGSGGGGSGGGGSAF
ELTQSPSSLSASVGDRVTIKCQASESISNYLSWYQQIPGKVPKLLIYYASNLASGVPSRFSGSGSGTDFT
LTISSLQPEDVATYYCQSYYGGGSAYTFGGGTKVEIK
SEQ ID 91: CT4 VH nt
CAGGTGCAGCTGGTGGAGTCTGGGGGAGGCGTGGTCCAGCCTGGGAGGTCCCTGAGACTCTCCTGTGCAGCCTCTGGATTCA
CCTTCAGTAGCTATACTATGCACTGGGTCCGCCAGGCTCCAGGCAAGGGGCTGGAGTGGGTGACATTTATATCATATGAT
GGAAACAATAAATACTACGCAGACTCCGTGAAGGGCCGATTCACCATCTCCAGAGACAATTCCAAGAACACGCTGTATCT
GCAAATGAACAGCCTGAGAGCTGAGGACACGGCTATATATTACTGTGCGAGGACCGGCTGGCTGGGGCCCTTTGACTACT
GGGGCCAGGGAACCCTGGTCACCGTCTCCTCA
SEQ ID 92: CT4 VH aa
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSS
SEQ ID 93: CT4 VL nt
GAAATTGTGTTGACGCAGTCTCCAGGCACCCTGTCTTTGTCTCCAGGGGAAAGAGCCACCCTCTCCTGCA
GGGCCAGTCAGAGTGTTGGCAGCAGCTACTTAGCCTGGTACCAGCAGAAACCTGGCCAGGCTCCCAGGCT
CCTCATCTATGGTGCATTCAGCAGGGCCACTGGCATCCCAGACAGGTTCAGTGGCAGTGGGTCTGGGACA
GACTTCACTCTCACCATCAGCAGACTGGAGCCTGAAGATTTTGCAGTGTATTACTGTCAGCAGTATGGTA
GCTCACCGTGGACGTTCGGCCAAGGGACCAAGGTGGAAATCAAA
SEQ ID 94: CT4 VL aa
EIVLTQSPGTLSLSPGERATLSCRASQSVGSSYLAWYQQKPGQAPRLLIYGAFSRATGIPDRFSGSGSGT
DFTLTISRLEPEDFAVYYCQQYGSSPWTFGQGTKVEIK
SEQ ID 95: PD224D1 VH nt
GAGGTGCAGCTGGTGGAGTCTGGGGGAGGCTTGGTCCAGCCTGGGGGGTCCCTGAGACTCTCCTGTACAG
CCTCTGGATTCTCCCTCAGTAGCTATGCAATGAGCTGGGTCCGCCAGGCTCCAGGGAAGGGGCTGGAGTA
CATCGGCTACATTGGTGATACTACTGGCATAGCCTACGCGAGCTGGGCGAATGGCAGATTCACCATCTCC
AAAGACAATACCAAGAACACGGTGGATCTTCAAATGAACAGCCTGAGAGCCGAGGACACGGCTGTGTATT
ACTGTGCGAGAGGCTGGTCCTACTTAGACATCTGGGGCCAAGGGACCCTGGTCACCGTCTCGAGC
SEQ ID 96: PD224D1 VH aa
EVQLVESGGGLVQPGGSLRLSCTASGFSLSSYAMSWVRQAPGKGLEYIGYIGDTTGIAYASWANGRFTIS
KDNTKNTVDL
QMNSLRAEDTAVYYCARGWSYLDIWGQGTLVTSS
SEQ ID 97: PD224D1 VL nt
GCCCTTGTGATGACCCAGTCTCCATCCTCCCTGTCTGCATCTGTAGGAGACAGAGTCACCATCACTTGCC
AGGCCAGTCAGAACATTTACAGCAATTTAGCCTGGTATCAGCAGAAACCAGGGAAAGTTCCTAAGCTCCT
GATCTATCAGGCCTCCACTCTGGCATCTGGGGTCCCATCTCGGTTCAGTGGCAGTGGATATGGGACAGAT
TTCACTCTCACCATCAGCAGCCTGCAGCCTGAAGATGTTGCAACTTATTACTGTCAAGGCGGTTATTATA
GTGCTGCCCTTAATACTTTCGGCGGAGGGACCAAGGTGGAGATCAAA
SEQ ID 98: PD224D1 VL aa
ALVMTQSPSSLSASVGDRVTITCQASQNIYSNLAWYQQKPGKVPKLLIYQASTLASGVPSRFSGSGYGTD
FTLTISSLQP
EDVATYYCQGGYYSAALNTFGGGTKVEIK
SEQ ID 99: PD206F12 VH nt
GAGGTGCAGCTGGTGGAGTCTGGGGGAGGCTTGGTCCAGCCTGGGGGGTCCCTGAGACTCTCCTGTGCAG
CCTCTGGATTCGACTTCAGTAGCGGCTACTGGATATGCTGGGTCCGCCAGGCTCCAGGGAAGGGGCTGGA
GTTGATCGCATGCATTTATGCTGGTACTAGTGGTAGTACTTCCTACGCGAGCTGGGCGAGAGGCAGATTC
ACCATCTCCGAAACCTCCAAGAACACGGTGACTCTTCAAATGAACAGCCTGAGAGCCGAGGACTCGGCTG
TGTATTACTGTGCGAGAAATCTTTACACTTACAATAGCTTGTGGGGCCAGGGAACCCTGGTCACCGTCTC
GAGC
SEQ ID 100: PD206F12 VH aa
EVQLVESGGGLVQPGGSLRLSCAASGFDFSSGYWICWVRQAPGKGLELIACIYAGTSGSTSYASWARGRF
TISETSKNTVTLQMNSLRAEDSAVYYCARNLYTYNSLWGQGTLVTVSS
SEQ ID 101: PD206F12 VL nt
GACATCCAGATGACCCAGTCTCCTTCCACCCTGTCTGCATCTGTAGGAGACAGAGTCACCATCACTTGCC
AGTCCAGTCAGAGTGTTTATGATAACAACTGGTTAGCCTGGTATCAGCAGAAACCAGGGAAAGCCCCTAA
GCTCCTGATCTATACAGTATCCACTCTGGCATCTGGGGTCCCATCAAGGTTCAGCGGCAGTGGATCTGGG
ACAGAATTCACTCTCACCATCAGCAGCCTGCAGCCTGATGATTTTGCAACTTATTACTGCCAAGGCACTT
ATTATAGTAGTGGTTGGAACTTTGCTTTCGGCGGAGGGACCAAGGTGGAGATCAAA
SEQ ID 102: PD206F12 VL aa
DIQMIQSPSTLSASVGDRVTITCQSSQSVYDNNWLAWYQQKPGKAPKLLIYTVSTLASGVPSRFSGSGSG
TEFTLTISSLQPDDFATYYCQGTYYSSGWNFAFGGGTKVEIK
SEQ ID 103: PD215A1 VH nt
CAGGAGCAGCTGTTGGAGTCTGGGGGAGGCTTGGTACAGCCTGGGGGGTCCCTGAGACTCTCCTGTACAG
CCTCTGGATTCTCCTTTAGCAGCTACTGGATGTGCTGGGTCCGCCAGGCTCCAGGGAAGGGGCTGGAGTG
GATCGGATGCATTACGACTGGTAGTGGTAGCACTTACTACGCGAGCTGGGCGAAGCGCCGGTTCACCATC
TCCAAAGACAATTCCAAGAACACGGTGACTCTGCAAATGAACAGCCTGAGAGCCGAGGACACGGCCGTAT
ATTACTGTACGAGAGCATTTGACTTGTGGGGCCAGGGAACCCTGGTCACCGTCTCGAGC
SEQ ID 104: PD215A1 VH aa
QEQLLESGGGLVQPGGSLRLSCTASGFSFSSYWMCWVRQAPGKGLEWIGCITTGSGSTYYASWAKRRFTI
SKDNSKNIVTLQMNSLRAEDTAVYYCTRAFDLWGQGTLVTVSS
SEQ ID 105: PD215A1 VL nt
GACATCCAGATGACCCAGTCTCCATCCTCCCTGTCTGCATCTGTAGGAGACAGAGTCACCATCACTTGCC
AGGCCAGTCAGAGCATTTACAGCTACTTAAACTGGTATCAGCAGAAACCAGGGAAAGCCCCTAAGCTCCT
GATCTATGGTGCATCCAATCTGGCATCTGGGGTCCCATCAAGGTTCAGTGGCAGTGGATCTGGGACAGAT
TTCACTCTCACCATCAGCAGTCTGCAACCTGAAGATTTTGCAACTTACTACTGTCAAAGCAGTTGGTTGA
GTGGTGCTGTTGGTAATGCTTTCGGCGGAGGGACCAAGGTGGAGATCAAA
SEQ ID 106: PD215A1 VL aa
DIQMTQSPSSLSASVGDRVTITCQASQSIYSYLNWYQQKPGKAPKLLIYGASNLASGVPSRFSGSGSGTD
FTLTISSLQPEDFATYYCQSSWLSGAVGNAFGGGTKVEIK
SEQ ID 107: PD225G11 VH nt
GAGGTGCAGCTGTTGGAGTCTGGGGGAGGCTTGGTACAGCCTGGGGGGTCCCTGAGACTCTCCTGTGCAG
CCTCTGGATTCACCTTTAGCAGCAGCCACTGGATATGCTGGGTCCGCCAGGCTCCAGGGAAGGGGCTGGA
GTGGATCGCATGCATTTATACTGGTAGTATTGATGTCTTTTACTACGCGAGCTGGGCGAAAGGCCGGTTC
ACCATCTCCAGAGACAATTCCAAGAACACGCTGTATCTGCAAATGAACAGCCTGAGAGCCGAGGACACGG
CCGTATATTACTGTGCGAGAGCCGCTAATACTGATACTACCTACTTTAACTTGTGGGGCCAGGGAACCCT
GGTCACCGTCTCGAGC
SEQ ID 108: PD225G11 VH aa
EVQLLESGGGLVQPGGSLRLSCAASGFTFSSSHWICWVRQAPGKGLEWIACIYTGSIDVFYYASWAKGRF
TISRDNSKNTLYLQMNSLRAEDTAVYYCARAANTDITYFNLWGQGTLVTVSS
SEQ ID 109: PD225G11 VL nt
GCCTATGATATGACCCAGTCTCCATCCTCCCTGTCTGCATCTGTAGGAGACAGAGTCACCATCAATTGCC
AGGCCAGTCAGAGCATTAACAACCAACTATCCTGGTATCAGCAGAAACCAGGGAAAGTTCCTAAGCTCCT
GATCTATGGTGCATCCACTCTGGCATCTGGGGTCCCATCTCGGTTCACCGGCAGTGGATCTGGGACAGAT
TTCACTCTCACCATCAGCAGCCTGCAGCCTGAAGATGTTGCAACTTATTACTGTCATGTTCATTATTGCA
GTGGTGGTAGTTGTTTTTGGGCTTTCGGCGGAGGGACCAAGGTGGAGATCAAA
SEQ ID 110: PD225G11 VL aa
AYDMTQSPSSLSASVGDRVTINCQASQSINNQLSWYQQKPGKVPKLLIYGASTLASGVPSRFTGSGSGTD
FTLTISSLQPEDVATYYCHVHYCSGGSCFWAFGGGTKVEIK
SEQ ID 111: PL23006 VH nt
CAGTCGGTGGAGGAGTCTGGGGGAGGCTTGGTCCAGCCTGGGGGGTCCCTGAGACTCTCCTGTACCGCCT
CTGGAATCGACCTTAATACCTACGACATGATCTGGGTCCGCCAGGCTCCAGGCAAGGGGCTAGAGTGGGT
TGGAATCATTACTTATAGTGGTAGTAGATACTACGCGAACTGGGCGAAAGGCCGATTCACCATCTCCAAA
GACAATACCAAGAACACGGTGTATCTGCAAATGAACAGCCTGAGAGCTGAGGACACGGCTGTGTATTACT
GTGCGAGAGATTATATGAGTGGTTCCCACTTGTGGGGCCAGGGAACCCTGGTCACCGTCTCTTCC
SEQ ID 112: PL23006 VH aa
QSVEESGGGLVQPGGSLRLSCTASGIDLNTYDMIWVRQAPGKGLEWVGIITYSGSRYYANWAKGRFTISK
DNTKNTVYLQMNSLRAEDTAVYYCARDYMSGSHLWGQGTLVTVSS
SEQ ID 113: PL23006 VL nt
GCCTATGATATGACCCAGTCTCCATCTTCCGTGTCTGCATCTGTAGGAGACAGAGTCACCATCAAGTGTC
AGGCCAGTGAGGACATTTATAGCTTCTTGGCCTGGTATCAGCAGAAACCAGGGAAAGCCCCTAAGCTCCT
GATCCATTCTGCATCCTCTCTGGCATCTGGGGTCCCATCAAGGTTCAGCGGCAGTGGATCTGGGACAGAT
TTCACTCTCACCATCAGCAGCCTGCAGCCTGAAGATTTTGCAACTTACTATTGTCAACAGGGTTATGGTA
AAAATAATGTTGATAATGCTTTCGGCGGAGGGACCAAGGTGGAGATCAAA
SEQ ID 114: PL23006 VL aa
AYDMTQSPSSVSASVGDRVTIKCQASEDIYSFLAWYQQKPGKAPKLLIHSASSLASGVPSRFSGSGSGTD
FTLTISSLQP
EDFATYYCQQGYGKNNVDNAFGGGTKVEIK
SEQ ID 115: PL231H2 VH nt
CAGGTGCAGCTGGTGGAGTCTGGGGGAGGCCTGGTCCAGCCTGGGGGCTCCCTGAGACTCTCCTGTACAA
CTTCTGGAATCGACCTTAGTACCTACGACATGATCTGGGTCCGCCAGGCTCCAGGCAAGGGGCTAGAGTG
GGTGGGAATCATTAGTTATGTTGGTAACACATACTACGCGAGCTGGGCGAAAGGCCGATTCACCCTCTCC
AAAGACAATACCTCGACCACGGTGGATCTGCAAATGAACAGCCTGAGAGCTGAGGACACGGCTGTGTATT
ACTGTGCGAGAGATTTTATTAGTGGTTCCCACTTGTGGGGCCAGGGAACCCTGGTCACCGTCTCGAGC
SEQ ID 116: PL231H2 VH aa
QVQLVESGGGLVQPGGSLRLSCTTSGIDLSTYDMIWVRQAPGKGLEWVGIISYVGNTYYASWAKGRFTLS
KDNTSTTVDLQMNSLRAEDTAVYYCARDFISGSHLWGQGTLVTVSS
SEA ID 117: PL231H2 VL nt
GCCTATGATATGACCCAGTCTCCATCTTCCGTGTCTGCATCTGTAGGAGACAGAGTCACCATCAATTGTC
AGGCCAGTGAGAGCATTAGTAGCTTCTTATCCTGGTATCAGCAGAAACCAGGGAAAGCCCCTAAGCTCCT
GATCTTTTCTGCATCCACTCTGGCATCTGGGGTCCCATCAAGGTTCAGCGGCAGTGGATCTGGGACAGAT
TTCACTCTCACCATCAGCAGCCTGCAGCCTGAAGATTTTGCAACTTACTATTGTCAACAGGGTTATAGTA
AAAGTAATGTGGATAATGCTTTCGGCGGAGGGACCAAGGTGGAGATCAAA
SEQ ID 118: PL231H2 VL aa
AYDMTQSPSSVSASVGDRVTINCQASESISSFLSWYQQKPGKAPKLLIFSASTLASGVPSRFSGSGSGTD
FTLTISSLQPEDFATYYCQQGYSKSNVDNAFGGGTKVEIK
SEQ ID 119: PL221G5 VH nt
GAGGTGCAGCTGTTGGAGTCTGGGGGAGGCTTGGTACAGCCTGGGGGGTCCCTGAGACTCTCCTGTGCAG
CCTCTGGATTCTCCTTCAGTAGCGGGTACGACATGTGCTGGGTCCGCCAGGCTCCAGGGAAGGGGCTGGA
GTGGATCGCATGCATTGCTGCTGGTAGTGCTGGTATCACTTACGACGCGAACTGGGCGAAAGGCCGGTTC
ACCATCTCCAGAGACAATTCCAAGAACACGCTGTATCTGCAAATGAACAGCCTGAGAGCCGAGGACACGG
CCGTATATTACTGTGCGAGATCGGCGTTTTCGTTCGACTACGCCATGGACCTCTGGGGCCAGGGAACCCT
GGTCACCGTCTCGAGC
SEA ID 120: PL221G5 VH aa
EVQLLESGGGLVQPGGSLRLSCAASGFSFSSGYDMCWVRQAPGKGLEWIACIAAGSAGITYDANWAKGRF
TISRDNSKNTLYLQMNSLRAEDTAVYYCARSAFSFDYAMDLWGQGTLVTVSS
SEQ ID 121: PL221G5 VL nt
GACATCCAGATGACCCAGTCTCCTTCCACCCTGTCTGCATCTGTAGGAGACAGAGTCACCATCACTTGCC
AGGCCAGTCAGAGCATTAGTTCCCACTTAAACTGGTATCAGCAGAAACCAGGGAAAGCCCCTAAGCTCCT
GATCTATAAGGCATCCACTCTGGCATCTGGGGTCCCATCAAGGTTCAGCGGCAGTGGATCTGGGACAGAA
TTTACTCTCACCATCAGCAGCCTGCAGCCTGATGATTTTGCAACTTATTACTGCCAACAGGGTTATAGTT
GGGGTAATGTTGATAATGTTTTCGGCGGAGGGACCAAGGTGGAGATCAAA
SEA ID 122: PL221G5 VL aa
DIQMTQSPSTLSASVGDRVTITCQASQSISSHLNWYQQKPGKAPKLLIYKASTLASGVPSRFSGSGSGTE
FTLTISSLQPDDFATYYCQQGYSWGNVDNVFGGGTKVEIK
SEA ID 123: PL004B5 VH nt
GAGGTGCAGCTGGTGGAGTCTGGGGGAGGCTTGGTCCAGCCTGGGGGGTCCCTGAGACTCTCCTGTGCAG
CCTCTGGATTCTCCCTCAGTAGCTACTGGATGAGCTGGGTCCGCCAGGCTCCAGGGAAGGGGCTGGAGTG
GATCGGAGTCATTGATACTAATGTTTATATATACTACGCGAACTGGGCAAAAGGCAGATTCACCATCTCC
AGAGACAATTCCAAGAACACGCTGTATCTTCAAATGAACAGCCTGAGAGCCGAGGACACGGCTGTGTATT
ACTGTGCGAGATATGTGGGTAATAATGATGATTATATTAACTTGTGGGGCCAGGGAACCCTGGTCACCGT
CTCGAGC
SEQ ID 124: PL004B5 VH aa
EVQLVESGGGLVQPGGSLRLSCAASGFSLSSYWMSWVRQAPGKGLEWIGVIDTNVYIYYANWAKGRFTIS
RDNSKNTLYLQMNSLRAEDTAVYYCARYVGNNDDYINLWGQGTLVTVSS
SEQ ID 125: PL004B5 VL nt
GACATCCAGATGACCCAGTCTCCTTCCACCCTGTCTGCATCTGTAGGAGACAGAGTCACCATCACTTGCC
AGTCCAGTCAGAGTGTTTATAATGGCTACTGGTTATCCTGGTATCAGCAGAAACCAGGGAAAGCCCCTAA
GCTCCTGATCTATGGTGCATCCACTCTGGCATCTGGGGTCCCATCAAGGTTCAGCGGCAGTGGATCTGGG
ACAGAATTCACTCTCACCATCAGCAGCCTGCAGCCTGATGATTTTGCAACTTATTACTGCCTAGGCAGTT
ATACTAGTAGTACTGAGAACTCTTTCGGCGGAGGGACCAAGGTGGAGATCAAA
SEQ ID 126: PL004B5 VL aa
DIQMTQSPSTLSASVGDRVTITCQSSQSVYNGYWLSWYQQKPGKAPKLLIYGASTLASGVPSRFSGSGSG
TEFTLTISSLQPDDFATYYCLGSYTSSTENSFGGGTKVEIK
SEA ID 127: PL004B9 VH nt
GAGGTGCAGCTGGTGGAGTCTGGGGGAGGCTTGGTCCAGCCTGGGGGGTCCCTGAGACTCTCCTGTACAG
TGTCTGGAATCGACCTCAGTGTCATCAATATGGGCTGGGTCCGCCAGGCTCCAGGGAAGGGGCTGGAGTG
GATCGGAACCATTACTTATGTTGGTAACACATATTACGCGAGCTGGGCGAAAGGCAGATTCACCATCTCC
AAAACCTCGACCACGGTGGATCTTAAAATCACCAGTCCGACAACCGAGGACACGGCTGTGTATTACTGTG
CGAGAGAATCTGGTACTATTTATTACAGTTACTTTAACTTGTGGGGCCAAGGGACCCTGGTCACCGTCTC
GAGC
SEQ ID 128: PL004B9 VH aa
EVQLVESGGGLVQPGGSLRLSCTVSGIDLSVINMGWVRQAPGKGLEWIGTITYVGNTYYASWAKGRFTIS
KTSTTVDLKITSPTTEDTAVYYCARESGTIYYSYFNLWGQGTLVTVSS
SEQ ID 129: PL004B9 VL nt
GCATTCGAATTGACCCAGTCTCCATCCTCCCTGTCTGCATCTGTAGGAGACAGAGTCACCATCAAGTGCC
AGGCCAGTGAAAGCATTAGCAACTACTTATCCTGGTATCAGCAGATTCCAGGGAAAGTTCCTAAGCTCCT
GATCTATTATGCATCCAATCTGGCATCTGGGGTCCCATCTCGGTTCAGTGGCAGTGGATCTGGGACAGAT
TTCACTCTCACCATCAGCAGCCTGCAGCCTGAAGATGTTGCAACTTATTACTGTCAAAGCTATTATGGTG
GTGGTAGTGCCTATACTTTCGGCGGAGGGACCAAGGTGGAGATCAAA
SEQ ID 130: PL004B9 VL aa
AFELTQSPSSLSASVGDRVTIKCQASESISNYLSWYQQIPGKVPKLLIYYASNLASGVPSRFSGSGSGTD
FTLTISSLQPEDVATYYCQSYYGGGSAYTFGGGTKVEIK
SEQ ID 131: PD220F6 VH nt
CAGGAGCAGGTGAAGGAGACCGGGGGAGGCTTGGTACAGCCTGGGGGGTCCCTGAGACTCTCCTGTGCAG
CCTCTGGATTCACCATCAGCAGCTATGGAGTGAGCTGGGTCCGCCAGGCTCCAGGGAAGGGGCTGGAGTG
GGTCGCATTGATTTTTCCCGGGATTGGTTTCAAAGACTACGCGAGCTGGGTGAATGGCCGGTTCACCCTC
TCCAGCGACAACGCCCAGAACACTGTGGAACTGCAAATGAACAGCCTGAGAGCCGAGGACACGGCCGTAT
ATTACTGTGCGAGAGATTTGGACTTGTGGGGCCAGGGAACCCTGGTCACCGTCTCGAGC
SEQ ID 132: PD220F6 VH aa
QEQVKETGGGLVQPGGSLRLSCAASGFTISSYGVSWVRQAPGKGLEWVALIFPGIGFKDYASWVNGRFTL
SSDNAQNTVELQMNSLRAEDTAVYYCARDLDLWGQGTLVTVSS
SEQ ID 133: PD220F6 VL nt
GACATCCAGATGACCCAGTCTCCATCCTCCCTGTCTGCATCTGTAGGAGACAGAGTCACCATCACTTGCC
AGTCCAGTCCGAGTGTTTATAGTAACTACTTATCCTGGTATCAGCAGAAACCAGGGAAAGTTCCTAAGCT
CCTGATCTATTATGCATCCACTCTGGCATCTGGGGTCCCATCTCGGTTCAGTGGCAGTGGATCTGGGACA
GATTTCACTCTCACCATCAGCAGCCTGCAGCCTGAAGATGTTGCAACTTATTACTGTGCAGGCGGTTATA
GTAGTAGTACTCGTGCTTTCGGCGGAGGGACCAAGGTGGAGATCAAA
SEQ ID 134: PD220F6 VL aa
DIQMTQSPSSLSASVGDRVTITCQSSPSVYSNYLSWYQQKPGKVPKLLIYYASTLASGVPSRFSGSGSGT
DFTLTISSLQPEDVATYYCAGGYSSSTRAFGGGTKVEIK
SEQ ID 135: human IgG1 constant region nt
GCTAGCACCAAGGGCCCATCGGTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTGGTGACC
GTGCCCTCCAGCAGCTTGGGCACCCAGACCTACATCTGCAACGTGAATCACAAGCCCAGCAACACCAAGG
TGGACAAGAGAGTTGAGCCCAAATCTTGTGACAAAACTCACACATGCCCACCGTGCCCAGCACCTGAACT
CCTGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTACACCC
TGCCCCCATCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAGCCGGAGAACAACTACAAGACCACGCCTCCCGTG
CTGGACTCCGACGGCTCCTTCTTCCTCTATAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTC
TCCGGGT
SEQ ID 136: human IgG1 constant region aa
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
SEQ ID 137: human IgG1 effector null nt
GCTAGCACCAAGGGCCCATCGGTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTGGTGACC
GTGCCCTCCAGCAGCTTGGGCACCCAGACCTACATCTGCAACGTGAATCACAAGCCCAGCAACACCAAGG
TGGACAAGAGAGTTGAGCCCAAATCTTGTGACAAAACTCACACATGCCCACCGTGCCCAGCACCTGAAGC
CGCGGGGGCACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCGCGGTCTCCAACAAAGCC
CTCCCAGCCCCCATCGAGAAAACCATCTCCAAAGCCAAAGGGCAGCCCCGAGAACCACAGGTGTACACCC
TGCCCCCATCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAGCCGGAGAACAACTACAAGACCACGCCTCCCGTG
CTGGACTCCGACGGCTCCTTCTTCCTCTATAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTC
TCCGGGT
SEQ ID 138: human IgG1 effector null aa
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPEAAGAPSVFLFPPKPKDTLMISRTP
EVTCVVVDVSHEDPEVKFNWYVDGVEVHNAKTKPREEQYNSTYRVVSVLTVLHQDWLNGKEYKCAVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
SEQ ID 139: PD220F6 scFv nt
CAGGAGCAGGTGAAGGAGACCGGGGGAGGCTTGGTACAGCCTGGGGGGTCCCTGAGACTCTCCTGTGCAG
CCTCTGGATTCACCATCAGCAGCTATGGAGTGAGCTGGGTCCGCCAGGCTCCAGGGAAGGGGCTGGAGTG
GGTCGCATTGATTTTTCCCGGGATTGGTTTCAAAGACTACGCGAGCTGGGTGAATGGCCGGTTCACCCTC
TCCAGCGACAACGCCCAGAACACTGTGGAACTGCAAATGAACAGCCTGAGAGCCGAGGACACGGCCGTAT
ATTACTGTGCGAGAGATTTGGACTTGTGGGGCCAGGGAACCCTGGTCACCGTCTCGAGCGGTGGAGGCGG
ATCTGGCGGAGGTGGTTCCGGCGGTGGCGGCTCCGGTGGAGGCGGCTCTGACATCCAGATGACCCAGTCT
CCATCCTCCCTGTCTGCATCTGTAGGAGACAGAGTCACCATCACTTGCCAGTCCAGTCCGAGTGTTTATA
GTAACTACTTATCCTGGTATCAGCAGAAACCAGGGAAAGTTCCTAAGCTCCTGATCTATTATGCATCCAC
TCTGGCATCTGGGGTCCCATCTCGGTTCAGTGGCAGTGGATCTGGGACAGATTTCACTCTCACCATCAGC
AGCCTGCAGCCTGAAGATGTTGCAACTTATTACTGTGCAGGCGGTTATAGTAGTAGTACTCGTGCTTTCG
GCGGAGGGACCAAGGTGGAGATCAAA
SEQ ID 140: PD220F6 scFv aa
QEQVKETGGGLVQPGGSLRLSCAASGFTISSYGVSWVRQAPGKGLEWVALIFPGIGFKDYASWVNGRFTL
SSDNAQNTVELQMNSLRAEDTAVYYCARDLDLWGQGTLVTVSSGGGGSGGGGSGGGGSGGGGSDIQMTQS
PSSLSASVGDRVTITCQSSPSVYSNYLSWYQQKPGKVPKLLIYYASTLASGVPSRFSGSGSGTDFTLTIS
SLQPEDVATYYCAGGYSSSTRAFGGGTKVEIK
SEQ ID 141: CT4 VH CDR1 aa
SSYTMH
SEQ ID 142: CT4 VH CDR2 aa
FISYDGNNKYYADSVKG
SEQ ID 143: CT4 VH CDR3 aa
TGWLGPFDY
SEQ ID 144: CT4 VL CDR1 aa
RASQSVGSSYLA
SEQ ID 145: CT4 VL CDR2 aa
GAFSRAT
SEQ ID 146: CT4 VL CDR3 aa
QQYGSSPWT
SEQ ID 147: PL23006 VH CDR1 aa
NTYDMI
SEQ ID 148: PL230C6 VH CDR2 aa
IITYSGSRYYANWAKG
SEQ ID 149: PL230C6 VH CDR3 aa
DYMSGSHL
SEQ ID 150: PL230C6 VL CDR1 aa
QASEDIYSFLA
SEQ ID 151: PL230C6 VL CDR2 aa
SASSLAS
SEQ ID 152: PL230C6 VL CDR3 aa
QQGYGKNNVDNA
SEQ ID 153: PD224D1 VH CDR1 aa
NTYDMT
SEQ ID 154: PD224D1 CDR2 aa
IITYSGSRYYANWAKG
SEQ ID 155: PD224D1 CDR3 aa
DYMSGSHL
SEQ ID 156: PD224D1 CDR1 aa
QASEDIYSFLA
SEQ ID 157: PD224D1 CDR2 aa
SASSLAS
SEQ ID 158: PD224D1 CDR3 aa
QQGYGKNNVDNA
SEQ ID 159: PL221G5 VH CDR1 aa
SGYDMC
SEQ ID 160: PL221G5 CDR2 aa
CIAAGSAGITYDANWAKG
SEQ ID 161: PL221G5 CDR3 aa
SAFSFDYAMDL
SEQ ID 162: PL221G5 CDR1 aa
QASQSISSHLN
SEQ ID 163: PL221G5 CDR2 aa
KASTLAS
SEQ ID 164: PL221G5 CDR3 aa
QQGYSWGNVDNV
SEQ ID 165: PL231H2 VH CDR1 aa
STYDMI
SEQ ID 166: PL231H2 VH CDR2 aa
IISYVGNTYYASWAKG
SEQ ID 167: PL231H2 VH CDR3 aa
DFISGSHL
SEQ ID 168: PL231H2 VL CDR1 aa
QASESISSFLS
SEQ ID 169: PL231H2 VL CDR2 aa
SASTLAS
SEQ ID 170: PL231H2 VL CDR3 aa
QQGYSKSNVDNA
SEQ ID 171: PL004B5 VH CDR1 aa
SSYWMS
SEQ ID 172: PL004B5 VH CDR2 aa
VIDTNVYIYYANWAKG
SEQ ID 173: PL004B5 VH CDR3 aa
YVGNNDDYINL
SEQ ID 174: PL004B5 VL CDR1 aa
QSSQSVYNGWLS
SEQ ID 175: PL004B5 VL CDR2 aa
GASTLAS
SEQ ID 176: PL004B5 VL CDR3 aa
LGSYTSSTENS
SEQ ID 177: PL004B9 VH CDR1 aa
SVINMG
SEQ ID 178: PL004B9 VH CDR2 aa
TITYVGNTYYASWAKG
SEQ ID 179: PL004B9 VH CDR3 aa
ESGTIYYSYFNL
SEQ ID 180: PL004B9 VL CDR1 aa
QASESISNYLS
SEQ ID 181: PL004B9 VL CDR2 aa
YASNLAS
SEQ ID 182: PL004B9 VL CDR3 aa
QSYYGGGSAYT
SEQ ID 183: PD206F12 VH CDR1 aa
SGYWIC
SEQ ID 184: PD206F12 VH CDR2 aa
CIYAGTSGSTSYASWARG
SEQ ID 185: PD206F12 VH CDR3 aa
NLYTYNSL
SEQ ID 186: PD206F12 VL CDR1 aa
QSSQSVYDNNWLA
SEQ ID 187: PD206F12 VL CDR2 aa
TVSTLAS
SEQ ID 188: PD206F12 VL CDR3 aa
QGTYYSSGWNFA
SEQ ID 189: PD215A1 VH CDR1 aa
SSYWMC
SEQ ID 190: PD215A1 VH CDR2 aa
CITTGSGSTYYASWAKR
SEQ ID 191: PD215A1 VH CDR3 aa
AFDL
SEQ ID 192: PD215A1 VL CDR1 aa
QASQSIYSYLN
SEQ ID 193: PD215A1 VL CDR2 aa
GASNLAS
SEQ ID 194: PD215A1 VL CDR3 aa
QSSWLSGAVGNA
SEQ ID 195: PD220F6 VH CDR1 aa
SSYGVS
SEQ ID 196: PD220F6 VH CDR2 aa
LIFPGIGFKDYASWVNG
SEQ ID 197: PD220F6 VH CDR3 aa
DLDL
SEQ ID 198: PD220F6 VL CDR1 aa
QSSPSVYSNYLS
SEQ ID 199: PD220F6 VL CDR2 aa
YASTLAS
SEQ ID 200: PD220F6 VL CDR3 aa
AGGYSSSTRA
SEQ ID 201: PD225G11 VH CDR1 aa
SSHWIC
SEQ ID 202: PD225G11 VH CDR2 aa
CIYTGSIDVFYYASWAKG
SEQ ID 203: PD225G11 VH CDR3 aa
AANTDTTYFNL
SEQ ID 204: PD225G11 VL CDR1 aa
QASQSINNQLS
SEQ ID 205: PD225G11 VL CDR2 aa
GASTLAS
SEQ ID 206: PD225G11 VL CDR3 aa
HVHYCSGGSCFWA

CDR Sequences for anti-CTLA4, anti-PD-1 and anti-PD-L1

| | | VH | | | VL | | |
|---|---|---|---|---|---|---|---|
| mAb | Target | CDR 1 | CDR 2 | CDR 3 | CDR 1 | CDR 2 | CDR 3 |
| CT4 | CTLA4 | SSYTMH | FISYDGNNKYYADSVKG | TGWLGPFDY | RASQSVGSSYLA | GAFSRAT | QQYGSSPWT |
| | | Seq ID | Seq ID 142 | Seq ID 143 | Seq ID 144 | Seq ID | Seq ID 146 |
| | | 141 | | | | 145 | |
| PL230C6 | PD-L1 | NTYDMI | IITYSGSRYYANWAKG | DYMSGSHL | QASEDIYSFLA | SASSLAS | QQGYGKNNVDNA |
| | | Seq ID | Seq ID 148 | Seq ID 149 | Seq ID 150 | Seq ID | Seq ID 152 |
| | | 147 | | | | 151 | |
| PD224D1 | PD-1 | SSYAMS | YIGDTTGIAYASWANG | GWSYLDI | QASQNIYSNLA | QASTLAS | QGGYYSAALNT |
| | | Seq ID | Seq ID 154 | Seq ID 155 | Seq ID 156 | Seq ID | Seq ID 158 |
| | | 153 | | | | 157 | |
| PL221G5 | PD-L1 | SGYDMC | CIAAGSAGITYDANWAKG | SAFSFDYAMDL | QASQSISSHLN | KASTLAS | QQGYSWGNVDNV |
| | | Seq ID | Seq ID 160 | Seq ID 161 | Seq ID 162 | Seq ID | Seq ID 164 |
| | | 159 | | | | 163 | |
| PL231H2 | PD-L1 | STYDMI | IISYVGNTYYASWAKG | DFISGSHL | QASESISSFLS | SASTLAS | QQGYSKSNVDNA |
| | | Seq ID | Seq ID 166 | Seq ID 167 | Seq ID 168 | Seq ID | Seq ID 170 |
| | | 165 | | | | 169 | |
| PL004B5 | PD-L1 | SSYWMS | VIDTNVYIYYANWAKG | YVGNNDDYINL | QSSQSVYNGWLS | GASTLAS | LGSYTSSTENS |
| | | Seq ID | Seq ID 172 | Seq ID 173 | Seq ID 174 | Seq ID | Seq ID 176 |
| | | 171 | | | | 175 | |
| PL004B9 | PD-L1 | SVINMG | TITYVGNTYYASWAKG | ESGTIYYSYFNL | QASESISNYLS | YASNLAS | QSYYGGGSAYT |
| | | Seq ID | Seq ID 178 | Seq ID 179 | Seq ID 180 | Seq ID | Seq ID 182 |
| | | 177 | | | | 181 | |
| PD206F12 | PD-1 | SGYWIC | CIYAGTSGSTSYASWARG | NLYTYNSL | QSSQSVYDNNWLA | TVSTLAS | QGTYYSSGWNFA |
| | | Seq ID | Seq ID 184 | Seq ID 185 | Seq ID 186 | Seq ID | Seq ID 188 |
| | | 183 | | | | 187 | |
| PD215A1 | PD-1 | SSYWMC | CITTGSGSTYYASWAKR | AFDL | QASQSIYSYLN | GASNLAS | QSSWLSGAVGNA |
| | | Seq ID | Seq ID 190 | Seq ID 191 | Seq ID 192 | Seq ID | Seq ID 194 |
| | | 189 | | | | 193 | |
| PD220F6 | PD-1 | SSYGVS | LIFPGIGFKDYASWVNG | DLDL | QSSPSVYSNYLS | YASTLAS | AGGYSSSTRA |
| | | Seq ID | Seq ID 196 | Seq ID 197 | Seq ID 198 | Seq ID | Seq ID 200 |
| | | 195 | | | | 199 | |
| PD225G11 | PD-1 | SSHWIC | CIYTGSIDVFYYASWAKG | AANTDTTYFNL | QASQSINNQLS | GASTLAS | HVHYCSGGSCFWA |
| | | Seq ID | Seq ID 202 | Seq ID 203 | Seq ID 204 | Seq ID | Seq ID 206 |
| | | 201 | | | | 205 | |

What is claimed is:

1. A bispecific antibody comprising IgG domains having heavy chains and light chains, and two scFv components being connected to either C terminal of the heavy chains or N terminal of the light chains, wherein the IgG domains have the binding specificity to a first antigen, wherein the scFv components have the binding specificity to a second antigen, and wherein the first antigen and the second antigen are different and are independently selected from α-CTLA4, α-PD-1, and α-PD-L1.

2. The bispecific antibody of claim 1, wherein the two scFv components are connected to the C terminal of the heavy chain.

3. The bispecific antibody of claim 2, wherein the first antigen comprises α-CTLA4 and the second antigen comprises α-PD-1 or α-PD-L1.

4. The bispecific antibody of claim 2, wherein the first antigen comprises α-PD-1 or α-PD-L1 and the second antigen comprises α-CTLA4.

5. The bispecific antibody of claim 1, wherein the two scFv components are connected to the N terminal of the light chain.

6. The bispecific antibody of claim 5, wherein the first antigen comprises α-PD-1 or α-PD-L1 and the second antigen comprises α-CTLA4.

7. The bispecific antibody of claim 5, wherein the first antigen comprises α-CTLA4 and the second antigen comprises α-PD-1 or α-PD-L1.

8. (canceled)

9. The bispecific antibody of claim 1, comprising an antigen-binding fragment having a sequence having at least 98% similarity with SEQ ID No. 92, 94, 96, 98, 100, 102, 104, 106, 108, 110, 112, 114, 116, 118, 120, 122, 124, 126, 128, 130, 132, or 134.

10-12. (canceled)

13. The bispecific antibody of claim 1, having a binding affinity to two of α-CTLA4, α-PD-1, or α-PD-L1 with a Kd not greater than 70 nM.

14. (canceled)

15. The bispecific antibody of claim 1, wherein the antibody is a humanized antibody, a chimeric antibody, or a recombinant antibody, or an isolated monoclonal antibody.

16. An IgG1 heavy chain for the bispecific antibody of claim 1, comprising an amino acid sequence having at least 98% similarity with SEQ ID No. 02, 06, 08, 10, 12, 14, 16, 18, 20, 22, 26, 30, 34, 38, 42, 46, 50, 54, 58, 62, 72, 92, 96, 100, 104, 108, 112, 116, 120, 124, 128, or 132.

17. A kappa light chain for the bispecific antibody of claim 1, comprising an amino acid sequence having at least 98% similarity with SEQ ID No. 04, 28, 32, 36, 40, 44, 48, 52, 56, 60, and 64.

18. A variable light chain for the bispecific antibody of claim 1, comprising an amino acid sequence having at least 98% similarity with SEQ ID No. 94, 98, 102, 106, 110, 114, 118, 122, 126, 130, or 134.

19. A variable heavy chain for the bispecific antibody of claim 1, comprising an amino acid sequence having at least 98% similarity with SEQ ID No. 92, 96, 100, 104, 108, 112, 116, 120, 124, 128, or 132.

20. An isolated nucleic acid encoding the bispecific antibody of claim 1.

21. An expression vector comprising the isolated nucleic acid of claim 20.

22-23. (canceled)

24. A host cell comprising the expression vector of claim 21, wherein the host cell is a prokaryotic cell or a eukaryotic cell.

25. (canceled)

26. A method of producing a bispecific antibody, comprising culturing the host cell of claim 24 so that the bispecific antibody is produced.

27-28. (canceled)

29. A pharmaceutical composition, comprising the bispecific antibody of claim 1 and a pharmaceutically acceptable carrier.

30-31. (canceled)

32. A method of treating a subject with a cancer, comprising administering to the subject an effective amount of the bispecific antibody of claim 1, wherein the cancer comprises cells expressing PD-L1.

33-39. (canceled)
